(19) United States
(12) Patent Application Publication
(10) Pub. No.: US 2010/0022547 A1
(43) Pub. Date: Jan. 28, 2010

(54) Compounds and Methods for Treating Mammalian Gastrointestinal Parasitic Infections

(75) Inventors: Lizbeth K. Hedstrom, Newton, MA (US); Boris Striepen, Athens, GA (US)

Correspondence Address: FOLEY HOAG, LLP;PATENT GROUP, WORLD TRADE CENTER WEST, 155 SEAPORT BLVD, BOSTON, MA, 02110, US

(73) Assignees: BRANDEIS UNIVERSITY, WALTHAM, MA (US); UNIVERSITY OF GEORFIA RESEARCH FOUNDATION, ATHENS, GA (US)

(21) Appl. No.: 12/302,600

(22) PCT Filed: Jun. 1, 2007
(86) PCT No.: PCT/US07/70233

§ 371 Date: Jun. 5, 2009

Related U.S. Application Data
(60) Provisional application No. 60/810,276, filed on Jun. 2, 2006.

(51) Int. Cl. A61K 31/498; A61K 31/165; C07C 233/01; A61K 31/426; C07D 277/20; A61K 31/502; C07D 237/30; A61K 31/47; C07D 215/00; A61K 31/519; C07D 495/02; A61K 31/4184; C07D 235/02; C07D 241/36; A61K 31/4433; C07D 405/02; A61P 33/02

(52) U.S. Cl. 514/249; 514/622; 564/175; 514/617; 564/180; 514/365; 548/202; 514/248; 544/237; 514/312; 546/156; 514/260.1; 544/278; 514/393; 548/302.1; 544/354; 514/338; 546/282.4

(57) ABSTRACT

One aspect of the present invention relates to compounds, and pharmaceutically acceptable salts and prodrugs thereof, that are useful as inhibitors of IMPDH. The invention also provides pharmaceutical compositions comprising the compounds of the invention which selectively inhibit parasitic IMPDH. In certain embodiments, the present invention relates to selective inhibition of C. parvum inosine-5′-monophosphate-dehydrogenase over human inosine-5′-monophosphate-dehydrogenase (IMPDH type I and type II). These compounds may be used alone or in combination with other therapeutic or prophylactic agents, such as anti-virals, anti-inflammatory agents, antimicrobials and immunosuppressants.

### RELATED APPLICATIONS

This application claims the benefit of priority to U.S. Provisional Patent Application Ser. No. 60/810,276, filed Jun. 2, 2006.

### GOVERNMENT SUPPORT

The invention was made with support provided by the National Institutes of Health (Grant No. NIAID AI55268); therefore, the government has certain rights in the invention.

### BACKGROUND OF THE INVENTION

Organisms must synthesize nucleotides in order for their cells to divide and replicate. Nucleotide synthesis in mammals may be achieved through one of two pathways: the de novo synthesis pathway or the salvage pathway. Different cell types use these pathways to differing extents.

Inosine-5′-monophosphate dehydrogenase (IMPDH; EC 1.1.1.205) is an enzyme involved in the biosynthesis of guanine nucleotides. IMPDH catalyzes the NAD-dependent oxidation of inosine-5′-monophosphate (IMP) to xanthosine-5′-monophosphate (XMP) [Jackson R. C. et. al., Nature, 256, pp. 331-333, (1975)]. Regardless of species, the reaction involves the random addition of substrates. A conserved active site Cys residue attacks the C2 position of IMP and hydride is transferred to NAD, producing NADH and the E-XMP* intermediate. NADH is released and a mobile flap folds into the vacant NADH site, E-XMP* hydrolyzes and XMP is released [W. Wang and L. Hedstrom, Biochemistry 36, pp. 8479-8483 (1997); J. Digits and L. Hedstrom, Biochemistry 38, pp. 2295-2306 (1999); Gan et al, Biochemistry 42, pp 847-863 (2003)]. The hydrolysis step is at least partially rate-limiting in all of the IMPDHs examined to date. The enzyme is unusual in that a large conformational change occurs in the middle of a catalytic cycle.

IMPDH is ubiquitous in eukaryotes, bacteria and protozoa [Y. Natsumeda & S. F. Carr, Ann. N.Y. Acad., 696, pp. 88-93 (1993)]. The prokaryotic forms share 30-40% sequence identity with the human enzyme. Two isoforms of human IMPDH, designated type I and type II, have been identified and sequenced [F. R. Collart and E. Huberman, J. Biol. Chem., 263, pp. 15769-15772, (1988); Y. Natsumeda et. al., J. Biol. Chem., 265, pp. 5292-5295, (1990)]. Each is 514 amino acids, and they share 84% sequence identity. Both IMPDH type I and type II form active tetramers in solution, with subunit molecular weights of 56 kDa [Y. Yamada et. al., Biochemistry, 27, pp. 2737-2745 (1988)].

The de novo synthesis of guanine nucleotides, and thus the activity of IMPDH, is particularly important in B- and T-lymphocytes. These cells depend on the de novo, rather than salvage pathway to generate sufficient levels of nucleotides necessary to initiate a proliferative response to mitogen or antigen [A. C. Allison et. al., Lancet II, 1179, (1975) and A. C. Allison et. al., Ciba Found. Symp., 48, 207, (1977)]. Thus, IMPDH is an attractive target for selectively inhibiting the immune system without also inhibiting the proliferation of other cells.

Immunosuppression has been achieved by inhibiting a variety of enzymes including, for example, the phosphatase calcineurin (inhibited by cyclosporin and FK-506); dihydroorotate dehydrogenase, an enzyme involved in the biosynthesis of pyrimidines (inhibited by leflunomide and brequinar); the kinase FRAP (inhibited by rapamycin); and the heat shock protein hsp70 (inhibited by deoxyspergualin). [See B. D. Kahan, Immunological Reviews, 136, pp. 29-49 (1993); R. E. Morris, The Journal of Heart and Lung Transplantation, 12(6), pp. S275-S286 (1993)].

Inhibitors of IMPDH are also known. U.S. Pat. Nos. 5,380,879 (incorporated by reference) and 5,444,072 (incorporated by reference) and PCT publications WO 94/01105 and WO 94/12184 describe mycophenolic acid (MPA) and some of its derivatives as potent, uncompetitive, reversible inhibitors of human IMPDH type I (Kᵢ=33 nM) and type II (Kᵢ=9 mM). MPA has been demonstrated to block the response of B- and T-cells to mitogen or antigen [A. C. Allison et. al., Ann. N.Y. Acad. Sci., 696, 63, (1993)].

Immunosuppressants, such as MPA, are useful drugs in the treatment of transplant rejection and autoimmune diseases. [R. E. Morris, Kidney Intl., 49, Suppl. 53, S-26, (1996)]. However, MPA is characterized by undesirable pharmacological properties, such as gastrointestinal toxicity and poor bioavailability. [L. M. Shaw, et. al., Therapeutic Drug Monitoring, 17, pp. 690-699, (1995)]

Nucleoside analogs such as tiazofurin, ribavirin and mizoribine also inhibit IMPDH [L. Hedstrom, et. al. Biochemistry, 29, pp. 849-854 (1990)]. These compounds require activation to either the adenine dinucleotide (tiazofurin) or monophosphate derivatives (ribavirin and mizoribine) that inhibit IMPDH. These activation pathways are often absent in the cell of interest. In addition, nucleoside analogs suffer from lack of selectivity and can be further metabolized to produce inhibitors of other enzymes. Therefore nucleoside analogs are prone to toxic side effects.

Mycophenolate mofetil, a prodrug which quickly liberates free MPA in vivo, was recently approved to prevent acute renal allograft rejection following kidney transplantation. [L. M. Shaw, et. al., Therapeutic Drug Monitoring, 17, pp. 690-699, (1995); H. W. Sollinger, Transplantation, 60, pp. 225-232 (1995)]. Several clinical observations, however, limit the therapeutic potential of this drug. [L. M. Shaw, et. al., Therapeutic Drug Monitoring, 17, pp. 690-699, (1995)]. MPA is rapidly metabolized to the inactive glucuronide in vivo. [A. C., Allison and E. M. Eugui, Immunological Reviews, 136, pp. 5-28 (1993)]. The glucuronide then undergoes enterohepatic recycling causing accumulation of MPA in the gastrointestinal tract where it cannot exert its IMPDH inhibitory activity on the immune system. This fact effectively lowers the drug's in vivo potency, while increasing its undesirable gastrointestinal side effects.

It is also known that IMPDH plays a role in other metabolic events. Increased IMPDH activity has been observed in rapidly proliferating human leukemic cell lines and other tumor cell lines, indicating IMPDH as a target for anti-cancer as well as immunosuppressive chemotherapy [M. Nagai et. al., Cancer Res., 51, pp. 3886-3890, (1991)]. IMPDH has also been shown to play a role in the proliferation of smooth muscle cells, indicating that inhibitors of IMPDH, such as MPA or rapamycin, may be useful in preventing restenosis or other hyperproliferative vascular diseases [C. R. Gregory et al., Transplantation, 59, pp. 655-61 (1995); PCT publication WO 94/12184; and PCT publication Wo 94/01105].

Additionally, IMPDH has been shown to play a role in viral replication in some viral cell lines. [S. F. Carr, J. Biol. Chem., 268, pp. 27286-27290 (1993)]. Analogous to lymphocyte and tumor cell lines, the implication is that the de novo, rather than the salvage, pathway is critical in the process of viral replication.

The IMPDH inhibitor ribavirin is used to treat viral diseases, including pneumonia in infants and children, hepatitis C virus (HCV) infection and is currently being evaluated for the treatment of hepatitis B virus (HBV) infection and disease. Ribavin enhances the sustained efficacy of interferon in HBV and HCV treatment. However, the therapeutic potential of ribavirin is limited by its lack of a sustained response in monotherapy and broad cellular toxicity.

Protozoan parasites are the causative agents of the world's most devastating diseases, including malaria, leishmaniasis and trypanosomiasis. Related protozoa cause a variety of diseases in immunocompromised patients, including toxoplasmosis and cryptosporidosis. Parasitic protozoa have complicated life cycles that may include cyst or resting stages, which are found throughout the environment, are resistant to variations in temperature and humidity, and exposure to many chemicals. People ingest these cysts, which then “hatch” in their bodies. Protozoans can be found in the intestines, lungs, muscle tissue and the digestive tract, releasing toxins and tissue-destroying enzymes. Protozoan infections may be associated with arthritis, asthma, degenerative muscle diseases, Hodgkin's disease, lymphoma, multiple sclerosis, ovarian cysts, psoriasis, cutaneous ulcers, dermatitis and others.

The protozoan parasite, Cryptosporidium parvum, is an important human pathogen causing gastrointestinal disease. Small children, pregnant women, the elderly, and immuno-compromised people (e.g., AIDS patients) are at risk of severe, chronic and often fatal infection. [Carey, C. M., Lee, H., and Trevors, J. T., Water Res., 38, 818-62 (2004); and Fayer, R., Veterinary Parasitology, 126, 37-56 (2004)]. The parasite produces spore-like oocysts that are highly resistant to water chlorination. Several large outbreaks in the U.S. have been linked to drinking and recreational water. Infection rates are extremely high, with disease manifest in 30% of exposed individuals and a 50-70% mortality rate among immuno-compromized individuals. Furthermore, there is a growing and credible concern that these organisms could be deliberately introduced into the water supply in an act of bioterrorism. Effective drugs are urgently needed for the management of cryptosporidiosis in AIDS patients and/or epidemic outbreaks.

All parasitic protozoa lack purine biosynthetic enzymes and must salvage purines from their hosts, making this pathway an extremely attractive target for developing anti-protozoal drugs. IMPDH is a key enzyme in the purine salvage pathway of C. parvum. As discussed above, IMPDH is a validated drug target in immunosuppressive, cancer and viral therapy, so the human enzymes are extremely well studied. It has recently been shown that C. parvum IMPDH has very different properties than the human enzymes and that IMPDH inhibitors block parasite proliferation in vivo [N. N. Umejiego et al, J Biol Chem, 279 pp. 40320-40327 (2004); and B. Striepen et al, Proc Natl Acad Sci USA, 101 pp. 3154-9 (2004)].

Thus, there exists a need for potent IMPDH inhibitors with improved pharmacological properties and selectivities. Such inhibitors should have therapeutic potential as immunosuppressants, anti-cancer agents, anti-vascular hyperproliferative agents, antiinflammatory agents, antifungal agents, antipsoriatic and anti-viral agents. Specifically, there is a need for selective IMPDH inhibitors that can slow or block parasite proliferation. The present invention fulfills this need and has other related advantages.

### SUMMARY OF THE INVENTION

One aspect of the present invention relates to compounds, and pharmaceutically acceptable salts and prodrugs thereof, that are useful as inhibitors of IMPDH. The invention also provides pharmaceutical compositions comprising the compounds of the invention which selectively inhibit parasitic IMPDH. In certain embodiments, the present invention relates to selective inhibition of C. parvum inosine-5′-monophosphate-dehydrogenase over human inosine-5′-monophosphate-dehydrogenase (IMPDH type I and type II). These compounds may be used alone or in combination with other therapeutic or prophylactic agents, such as anti-virals, anti-inflammatory agents, antimicrobials and immunosuppressants.

### BRIEF DESCRIPTION OF THE FIGURES

FIG. 1 depicts selected compounds (A-H, J, and K) of the invention.

FIG. 2 depicts a graph and table showing selective inhibition of parasitic IMPDH by compounds A-H, J, and K. Key: a. ≦30% inhibition is observed at 50 μM inhibitor except as noted. b. ≦20% inhibition is observed at 50 μM inhibitor except as noted. c. 40% inhibition at 25 μM d. 45% inhibition at 25 μM.

FIG. 3 tabulates data relating to growth inhibition and cytotoxcity. Cytotoxicity was assessed in two assays: (1) % cyto-lysis was determined by measuring the release of LDH using CytoTox assay (Promega); and (2) Cytostatic effects were evaluated with the LIVE/DEAD assay (Molecular probes).

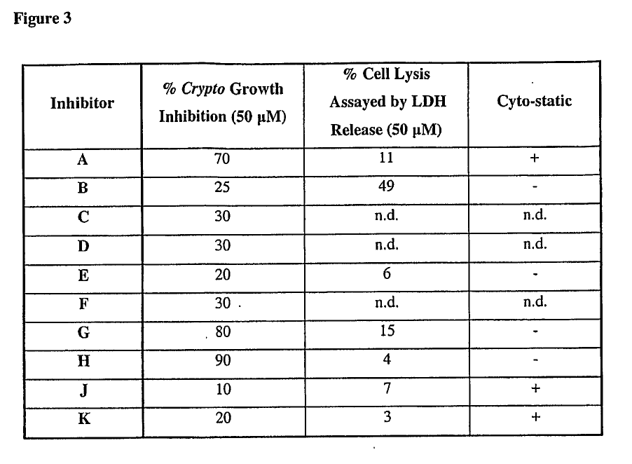

FIG. 4 depicts activity of compounds A-H, J, and K in a cell culture model of C. parvum infection. Parasite growth was monitored with (A) a cell-based ELISA using biotin-conjugated to the Vicia villosa-lectin (VVL) to measure parasite growth to measure parasite growth and (B) a real-time PCR assay. The PCR assay specifically measures the abundance of parasite ribosomal RNA genes while the VVL lectin preferentially recognizes a single alpha N-acetylgalactosamine residue linked to serine or threonine and binds C. parvum sporozoites, intracellular stages of the parasite, the inner oocyst wall, but not the outer oocyst wall; the observed activity does not result from cytotoxic or cytostatic effects on the host cells.

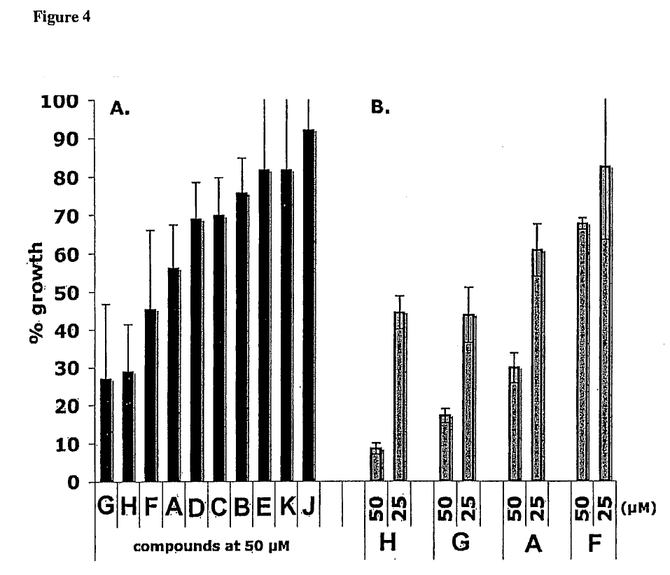

FIG. 5 depicts selected compounds (A-A5) of the invention and their IC₅₀ values against C. parvum IMPDH.

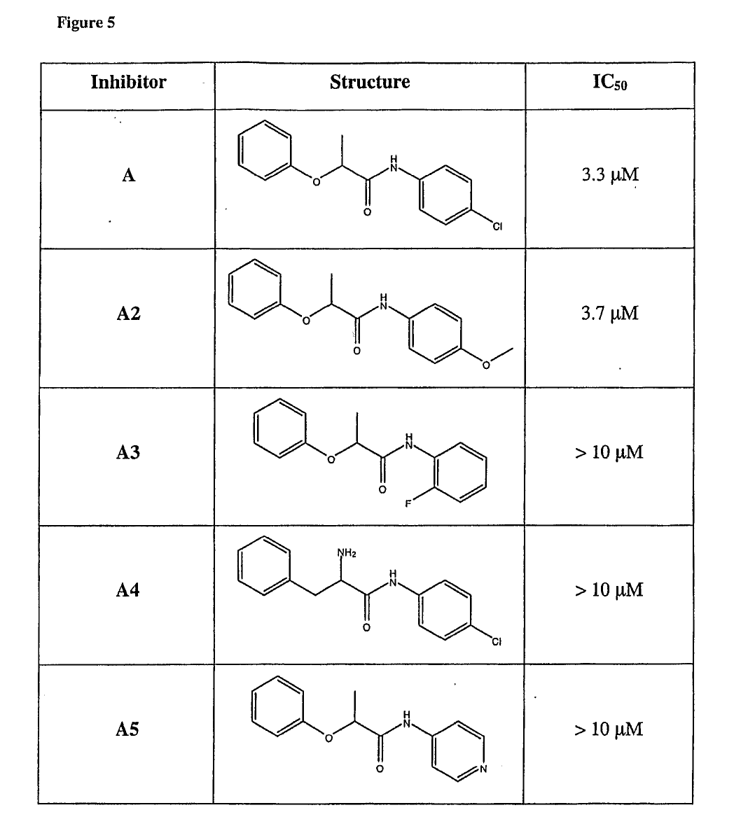

FIG. 6 depicts selected compounds (B-B5) of the invention and their IC₅₀ values against C. parvum IMPDH.

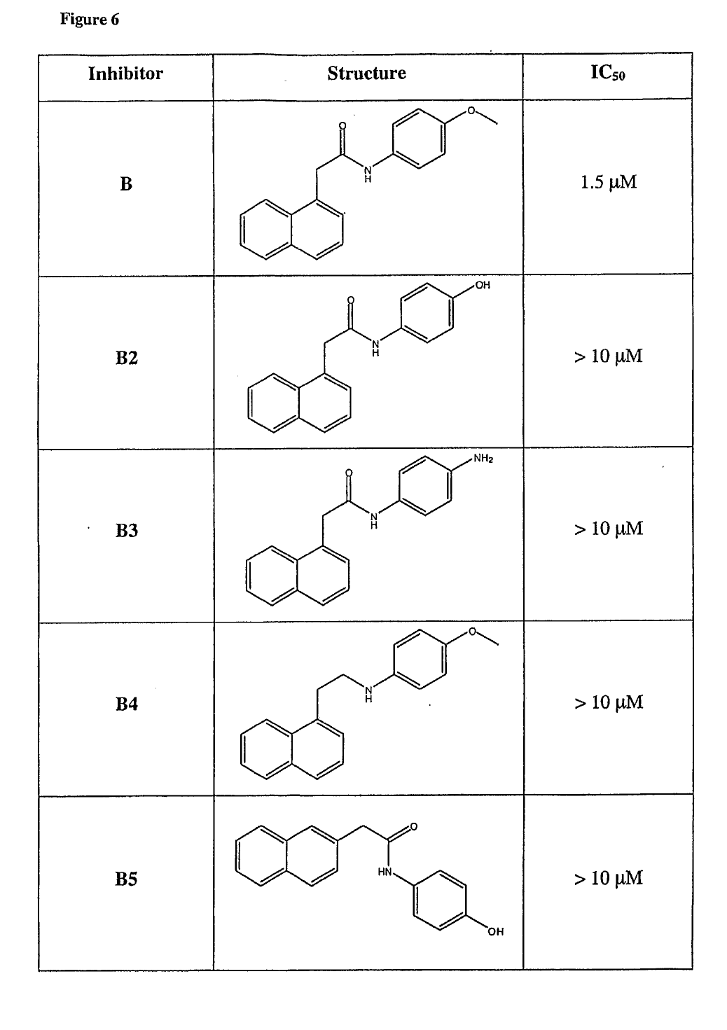

FIG. 7 depicts selected compounds (B6-B9) of the invention and their IC₅₀ values against C. parvum IMPDH.

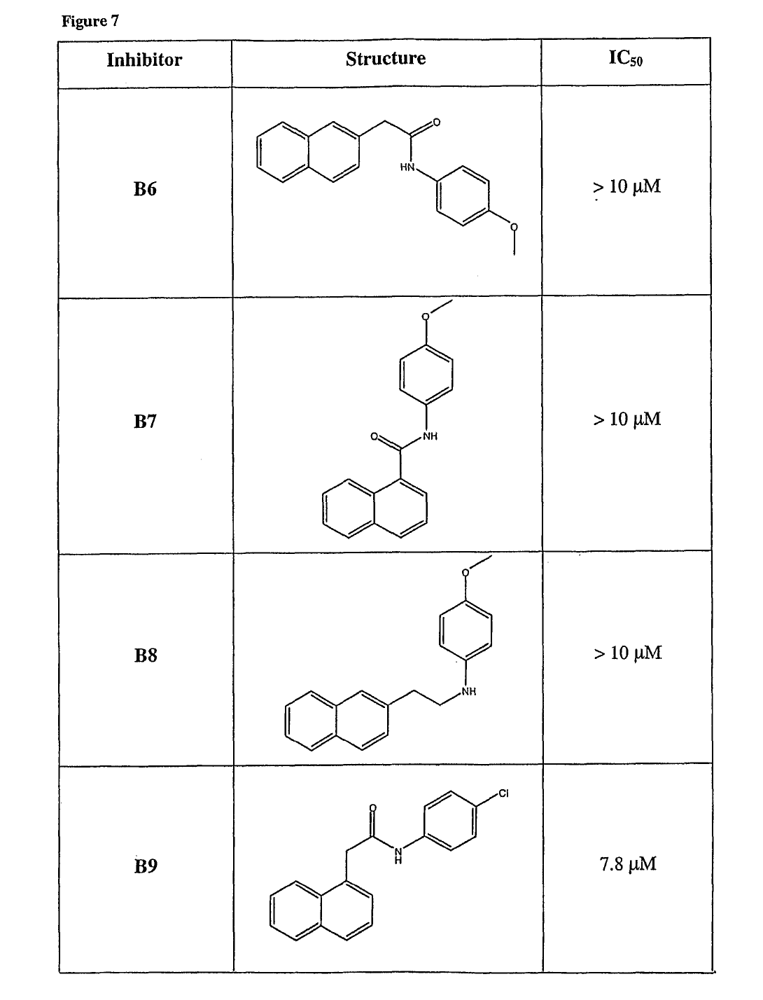

FIG. 8 depicts selected compounds (G-G7) of the invention and their IC₅₀ values against C. parvum IMPDH.

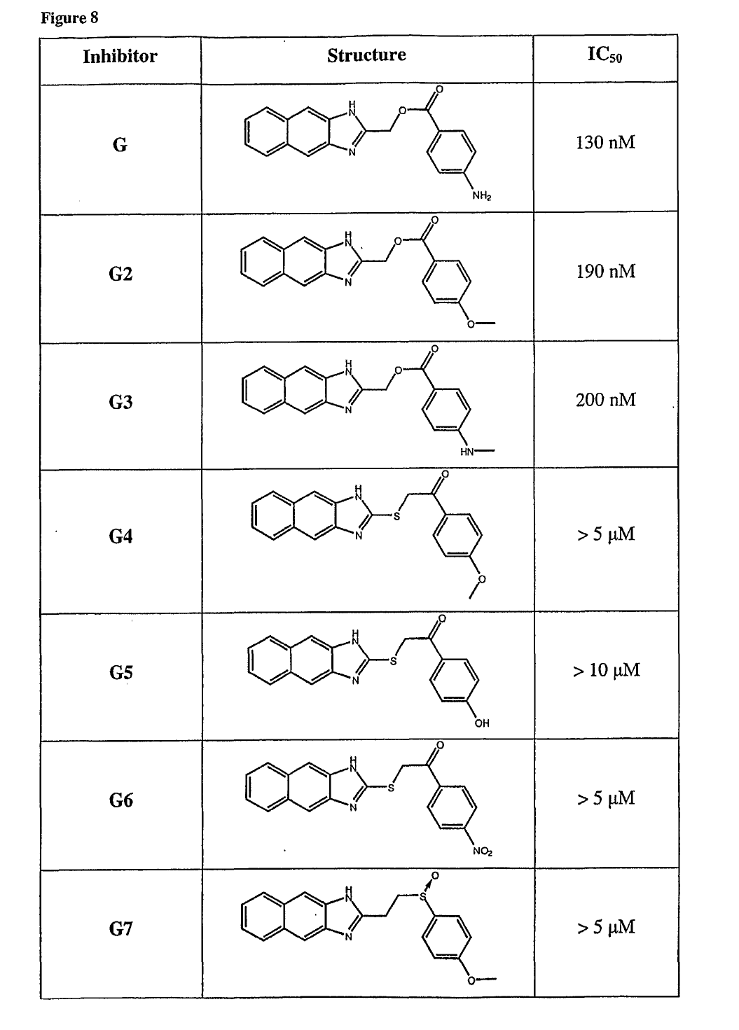

### DETAILED DESCRIPTION

One aspect of the present invention relates to compounds, and pharmaceutically acceptable salts and prodrugs thereof, that are useful as inhibitors of inosine-5′-monophosphate-dehydrogenase (IMPDH). The invention also provides pharmaceutical compositions comprising a compound of the invention which selectively inhibits parasitic IMPDH. In certain embodiments, the present invention relates to selective inhibition of C. parvum IMPDH in the presence of human inosine-5′-monophosphate-dehydrogenase (IMPDH type I and type II).

IMPDH-Mediated Diseases. IMPDH-mediated disease refers to any disease state in which the IMPDH enzyme plays a regulatory role in the metabolic pathway of that disease. Examples of IMPDH-mediated disease include transplant rejection and autoimmune diseases, such as rheumatoid arthritis, multiple sclerosis, juvenile diabetes, asthma, and inflammatory bowel disease, as well as other inflammatory diseases, cancer, viral replication diseases and vascular diseases.

For example, the compounds, compositions and methods of using them of this invention may be used in the treatment of transplant rejection (e.g., kidney, liver, heart, lung, pancreas (islet cells), bone marrow, cornea, small bowel and skin allografts and heart valve xenografts) and autoimmune diseases, such as rheumatoid arthritis, multiple sclerosis, juvenile diabetes, asthma, inflammatory bowel disease (Crohn's disease, ulcerative colitus), lupus, diabetes, mellitus myasthenia gravis, psoriasis, dermatitis, eczema, seborrhoea, pulmonary inflammation, eye uveitis, hepatitis, Grave's disease, Hashimoto's thyroiditis, Behcet's or Sjorgen's syndrome (dry eyes/mouth), pernicious or immunohaemolytic anaemia, idiopathic adrenal insufficiency, polyglandular autoimmune syndrome, and glomerulonephritis, scleroderma, lichen planus, viteligo (depigmentation of the skin), autoimmune thyroiditis, and alveolitis, inflammatory diseases such as osteoarthritis, acute pancreatitis, chronic pancreatitis, asthma and adult respiratory distress syndrome, as well as in the treatment of cancer and tumors, such as solid tumors, lymphomas and leukemia, vascular diseases, such as restenosis, stenosis and artherosclerosis, and DNA and RNA viral replication diseases, such as retroviral diseases, and herpes.

Selective Inhibition of Microbial IMPDH. IMPDH enzymes are also known to be present in bacteria, fungi, and protozoans and thus may regulate microbial growth. As such, the IMPDH-inhibitor compounds, compositions and methods described herein may be useful as antibacterials, antifungals, and/or antiprotozoans, either alone or in combination with other anti-microbial agents.

Microbial inhibition can be measured by various methods, including, for example, IMPDH HPLC assays (measuring enzymatic production of XMP and NADH from IMP and NAD), IMPDH spectrophotometric assays (measuring enzymatic production of NADH from NAD or XMP from IMP), IMPDH fluormetric assays (measuring enzymatic production of NADH from NAD), IMPDH radioassays (measuring enzymatic production of radiolabeled XMP from radiolabeled IMP or tritium release into water from 2-³H-IMP). [See C. Montero et al., Clinica Chimica Acta, 238, pp. 169-178 (1995)]. Additional assays known in the art can be used in ascertaining the degree of activity of an inventive compound as an IMPDH inhibitor. For example, activity of IMPDH I and IMPDH II can be measured following an adaptation of the method described in WO 97/40028. [See, additionally, U.S. Patent Application 2004/0102497 (incorporated by reference)].

Accordingly, in certain embodiments, the inventive compounds are capable of targeting and selectively inhibiting the IMPDH enzyme in bacteria. It is known that knocking out the IMPDH gene makes some bacteria avirulent, while has no effect on others. The effectiveness probably depends on which salvage pathways are operational in a given bacteria, and the environmental niche of the infection. It has been shown that Salmonella, Shigella, Yersinia and Steprococcus may be sensitive to IMPDH inhibitors of the invention. In addition, Staphylococcus and Bacillus anthracis are sensitive to mycophenolic acid, suggesting that IMPDH inhibitors of the invention may also be effective against these bacteria. In addition, in certain embodiments, these compounds are capable of targeting and selectively inhibiting the IMPDH enzyme in fungi, as evidenced by the mycophenolic acid sensitivity of Saccharomyces cerevidiea, Candida albicans, Cryptococcus neoformans, Aspergillus flavus and Trichophyton.

Further, in certain embodiments, the inventive compounds are capable of targeting and selectively inhibiting the IMPDH enzyme in protozoans, such as Toxoplasma, Eimeria, Cryptosporidium, Plasmodium, Babesia, Theileria, Neospora, Sarcocystis, Giardia, Entamoeba, Trichomonas, Leishmania and Trypanosoma. In certain embodiments, these compounds are capable of targeting and selectively inhibiting the IMPDH enzyme in Cryptosporidium parvum.

Selected Compounds of the Invention. One aspect of the invention relates to a compound, or a pharmaceutically acceptable salt, derivative or prodrug thereof, selected

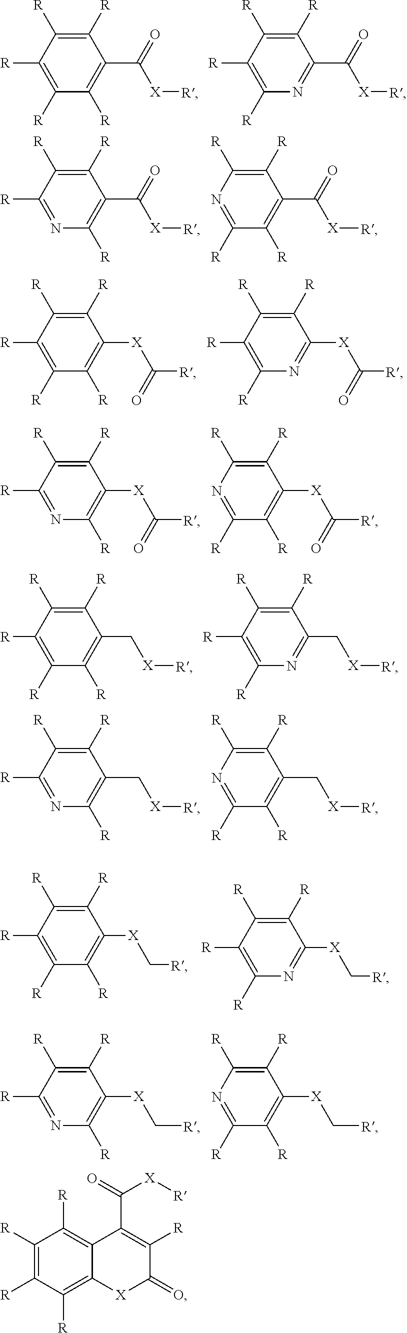

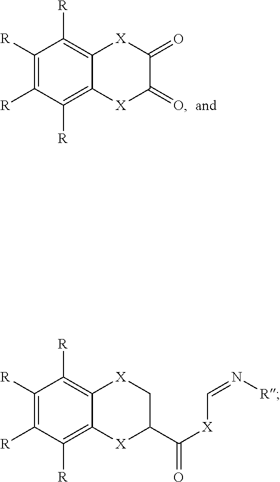

from the group consisting of

wherein, independently for each occurrence,

X is —CH₂—, —N(R^{N})—, —O—, —S(═O)₂—, —S(═O)—, or —S—;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C};

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano;

R′ is alkyl, heteroalkyl, cycloalkyl, alkcycloalkyl, alkoxycycloalkyl, alkaminocycloalkyl, alkthiocycloalkyl, heterocycloalkyl, alkylheterocycloalkyl, alkoxyheterocycloalkyl, alkaminoheterocycloalkyl, alkthioheterocycloalkyl, aryl, alkylaryl, alkoxyaryl, alkaminoaryl, alkthioaryl, heteroaryl, alkylheteroaryl, alkoxyheteroaryl, alkaminoheteroaryl, or alkthioheteroaryl,

R″ is selected from the group consisting of aryl or heteroaryl; and

any two adjacent R, taken together with the atoms to which they are directly bound, may form an optionally substituted, 5-membered or 6-membered, saturated or unsaturated, carbocyclic or heterocyclic, ring.

In certain embodiments, the present invention relates to the aforementioned compound, wherein X is —O—.

In certain embodiments, the present invention relates to the aforementioned compound, wherein X is —NH—

In certain embodiments, the present invention relates to the aforementioned compound, wherein X is —CH₂—.

In certain embodiments, the present invention relates to the aforementioned compound, wherein X is —S(═O)—.

In certain embodiments, the present invention relates to the aforementioned compound, wherein R is hydrogen, halogen, alkyl, hydroxyl, alkoxy, amino, nitro, sulfhydryl, imino, amido, alkylthio, sulfonyl, sulfonamido, trifluoromethyl, or cyano.

In certain embodiments, the present invention relates to the aforementioned compound, where in R′ is alkyl, heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned compound, wherein R″ is monocyclic.

In certain embodiments, the present invention relates to the aforementioned compound, wherein said compound is selected from the group consisting of

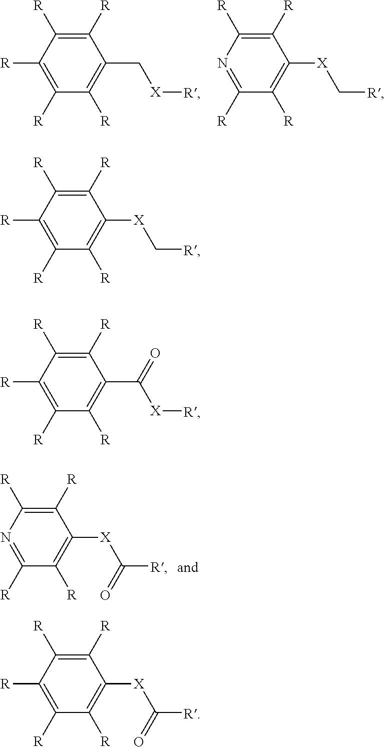

In certain embodiments, the present invention relates to the aforementioned compound, wherein X is —O—.

In certain embodiments, the present invention relates to the aforementioned compound, wherein X is —NH—

In certain embodiments, the present invention relates to the aforementioned compound, wherein X is —CH₂—.

In certain embodiments, the present invention relates to the aforementioned compound, wherein X is —S(═O)—.

In certain embodiments, the present invention relates to the aforementioned compound, wherein R is hydrogen, halogen, hydroxyl, alkoxy, amino, or amido.

In certain embodiments, the present invention relates to the aforementioned compound, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned compound, wherein R′ is monocyclic, bicyclic or tricyclic.

In certain embodiments, the present invention relates to the aforementioned compound, wherein R′ is

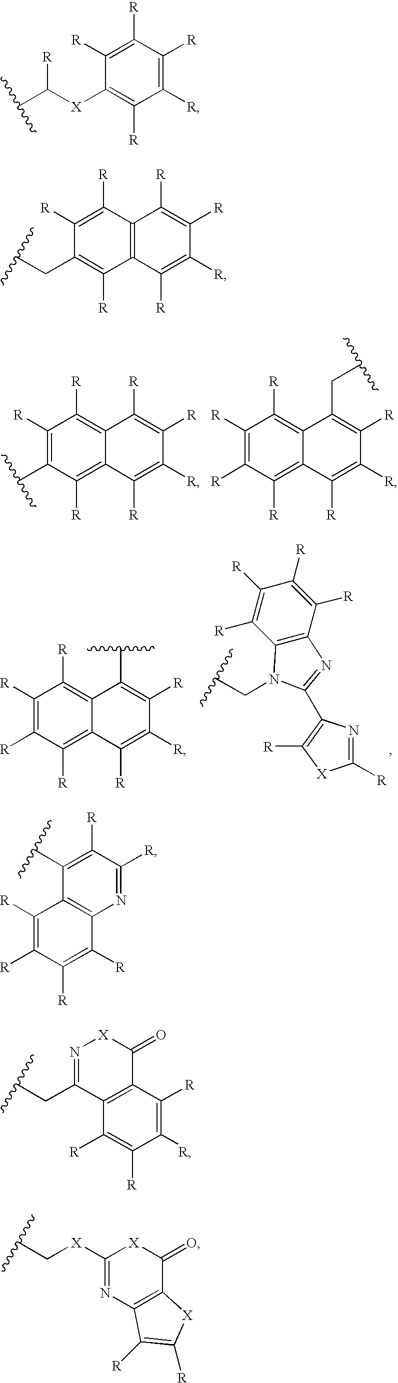

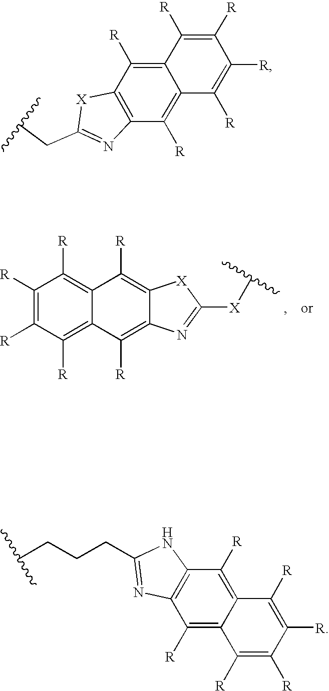

In certain embodiments, the present invention relates to the aforementioned compound, wherein said compound is selected from the group consisting of

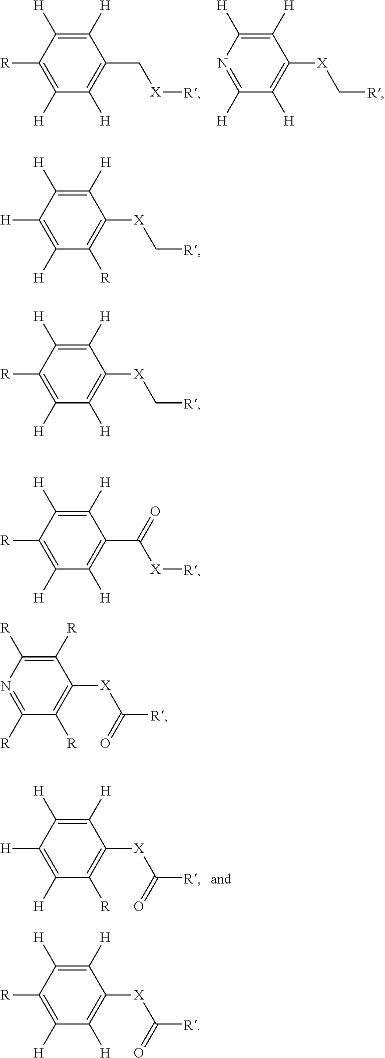

In certain embodiments, the present invention relates to the aforementioned compound, wherein X is —O—.

In certain embodiments, the present invention relates to the aforementioned compound, wherein X is —NH—

In certain embodiments, the present invention relates to the aforementioned compound, wherein X is —CH₂—.

In certain embodiments, the present invention relates to the aforementioned compound, wherein X is —S(═O)—.

In certain embodiments, the present invention relates to the aforementioned compound, wherein R is hydrogen, halogen, hydroxyl, alkoxy, nitro, amino, or amido.

In certain embodiments, the present invention relates to the aforementioned compound, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned compound, wherein R′ is monocyclic, bicyclic or tricyclic.

In certain embodiments, the present invention relates to the aforementioned compound, wherein R′ is

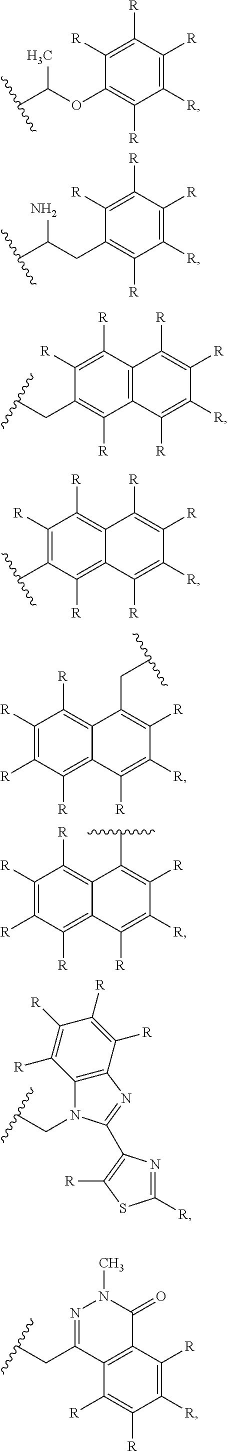

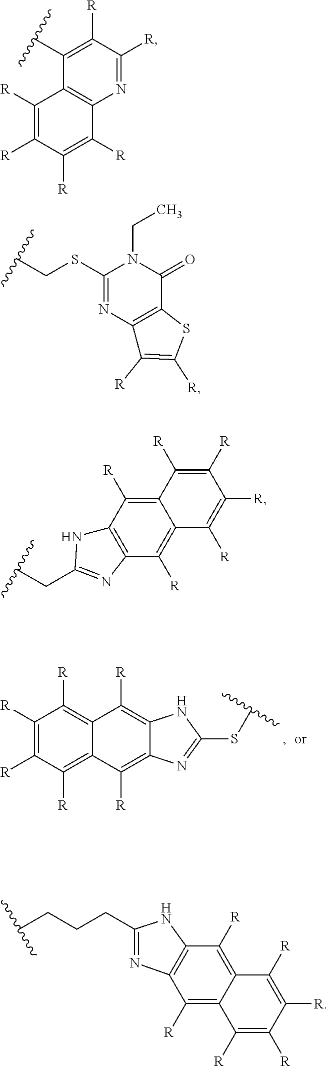

In certain embodiments, the present invention relates to the aforementioned compound, wherein said compound is selected from the group consisting of

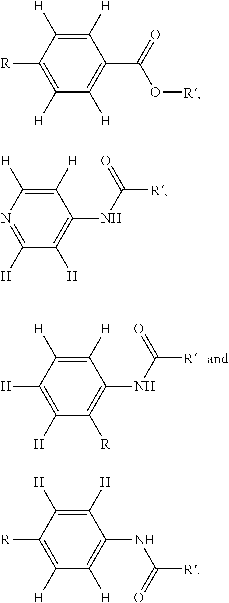

In certain embodiments, the present invention relates to the aforementioned compound, wherein R is —H, —Cl, —F, —OH, —NH(CH₃), —NO₂, —OCH₃, or —NH₂.

In certain embodiments, the present invention relates to the aforementioned compound, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned compound, wherein R′ is monocyclic, bicyclic or tricyclic.

In certain embodiments, the present invention relates to the aforementioned compound, wherein R′ is

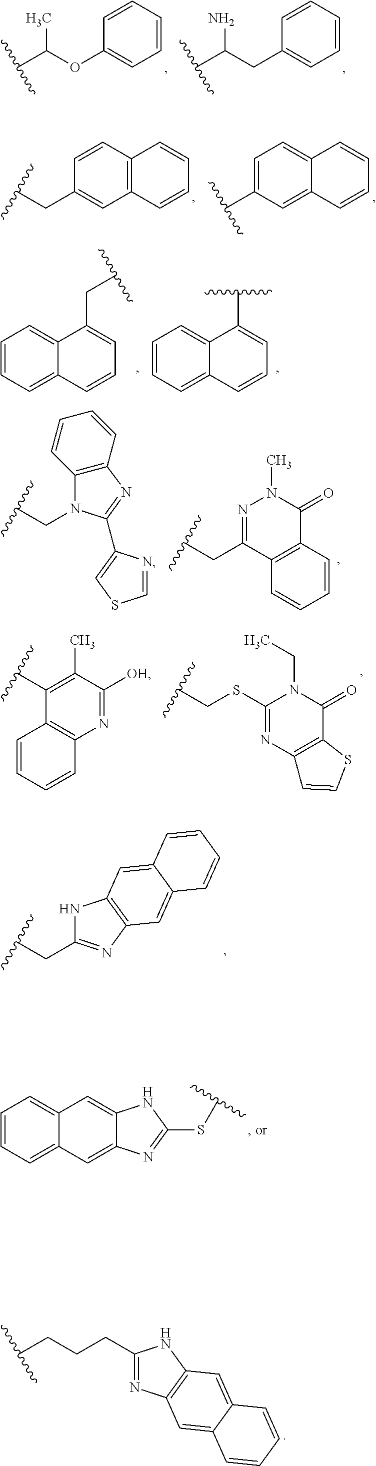

In certain embodiments, the present invention relates to the aforementioned compound, wherein said compound is selected from the group consisting

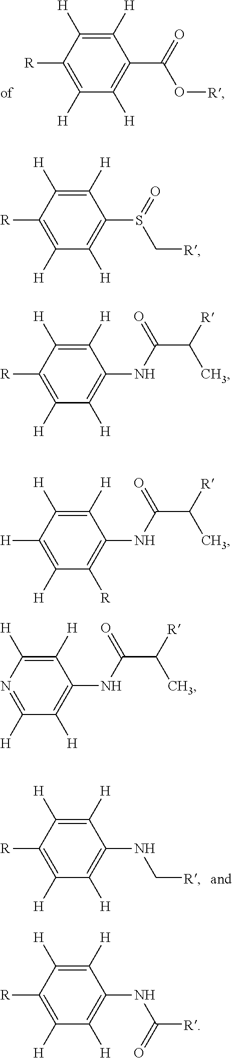

In certain embodiments, the present invention relates to the aforementioned compound, wherein R is —H, —Cl, —F, —OH, —NH(CH₃), —NO₂, —OCH₃, or —NH₂.

In certain embodiments, the present invention relates to the aforementioned compound, wherein R′ is heterocycloalkyl, aryl, oxyaryl, or heteroaryl.

In certain embodiments, the present invention relates to the aforementioned compound, wherein R′ is monocyclic, bicyclic or tricyclic.

In certain embodiments, the present invention relates to the aforementioned compound, wherein said compound is

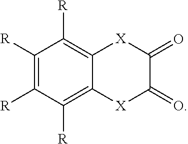

In certain embodiments, the present invention relates to the aforementioned compound, wherein X is —O—, or —NH—.

In certain embodiments, the present invention relates to the aforementioned compound, wherein said compound is

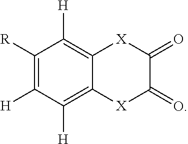

In certain embodiments, the present invention relates to the aforementioned compound, wherein X is —O—, or —NH—.

In certain embodiments, the present invention relates to the aforementioned compound, wherein X is —NH—.

In certain embodiments, the present invention relates to the aforementioned compound, wherein R is hydroxyl, alkoxy, aryloxy, heteroaryloxy, amino, alkylamino, arylamino, or heteroarylamino.

In certain embodiments, the present invention relates to the aforementioned compound, wherein R is

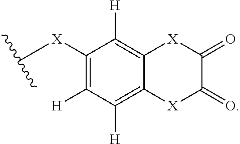

In certain embodiments, the present invention relates to the aforementioned compound, wherein X is —O—, or —NH—.

In certain embodiments, the present invention relates to the aforementioned compound, wherein R is

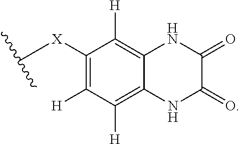

In certain embodiments, the present invention relates to the aforementioned compound, wherein X is —O—, or —NH—.

In certain embodiments, the present invention relates to the aforementioned compound, wherein said compound is

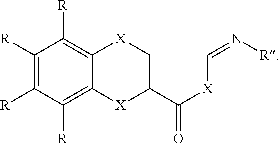

In certain embodiments, the present invention relates to the aforementioned compound, wherein X is —O—, or —NH—.

In certain embodiments, the present invention relates to the aforementioned compound, wherein R is hydrogen, halogen, hydroxyl, alkoxy, amino, or amido.

In certain embodiments, the present invention relates to the aforementioned compound, wherein R″ is monocyclic.

In certain embodiments, the present invention relates to the aforementioned compound, wherein said compound is

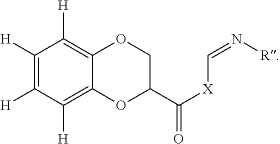

In certain embodiments, the present invention relates to the aforementioned compound, wherein X is —NH—.

In certain embodiments, the present invention relates to the aforementioned compound, wherein R″ is monocyclic.

In certain embodiments, the present invention relates to the aforementioned compound, wherein R is phenyl, or pyridine.

In certain embodiments, the present invention relates to the aforementioned compound, wherein said compound is

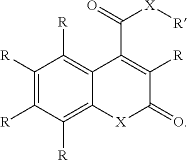

In certain embodiments, the present invention relates to the aforementioned compound, wherein X is —O—, or —NH—.

In certain embodiments, the present invention relates to the aforementioned compound, wherein R is hydrogen, halogen, hydroxyl, alkoxy, amino, or amido.

In certain embodiments, the present invention relates to the aforementioned compound, wherein R″ is alkyl.

In certain embodiments, the present invention relates to the aforementioned compound, wherein said compound is

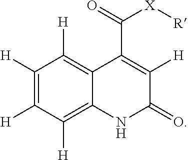

In certain embodiments, the present invention relates to the aforementioned compound, wherein X is —O—, or —NH—.

In certain embodiments, the present invention relates to the aforementioned compound, wherein R″ is alkyl.

In certain embodiments, the present invention relates to the aforementioned compound, excluding a compound selected from the group consisting of

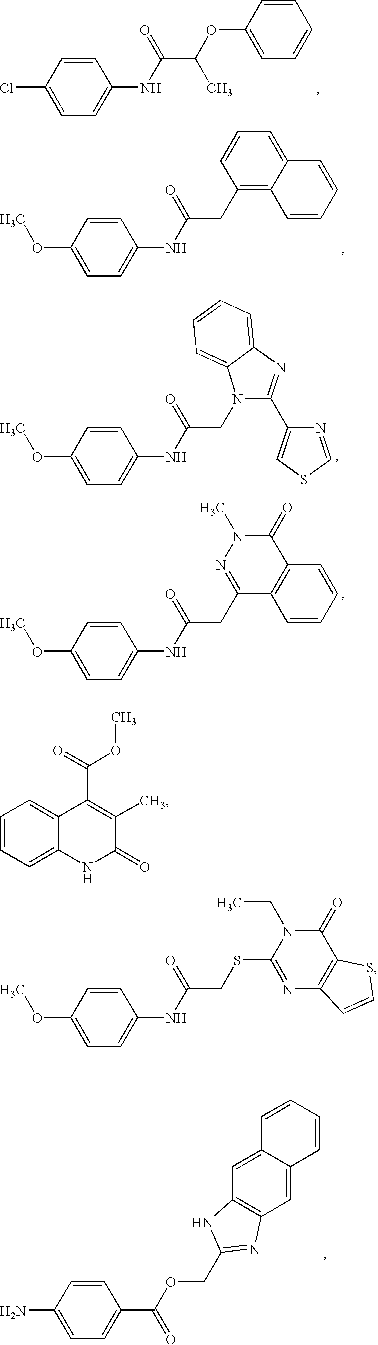

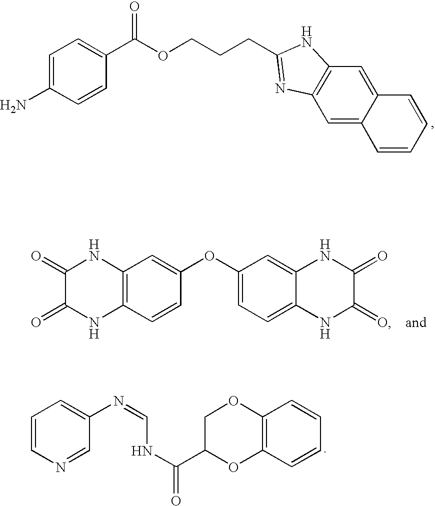

Another aspect of the invention relates to a compound, or a pharmaceutically acceptable salt, derivative or prodrug thereof, selected from the group consisting of

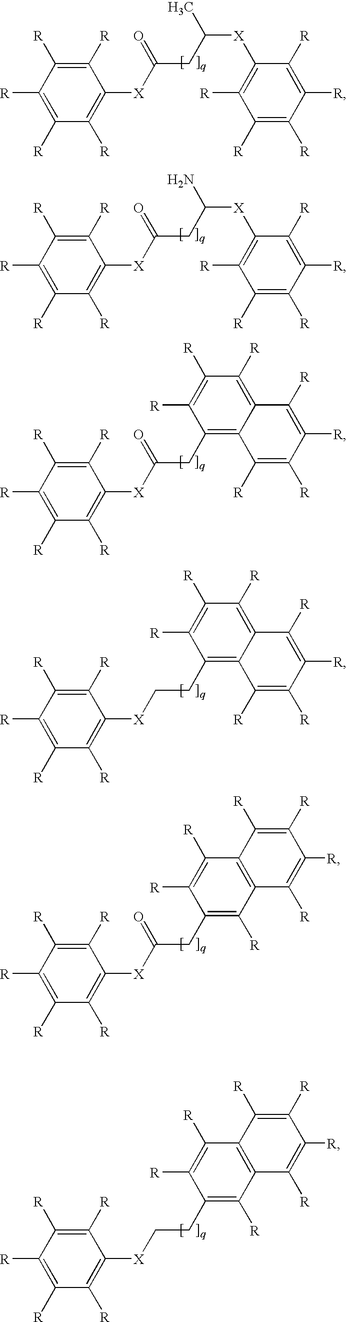

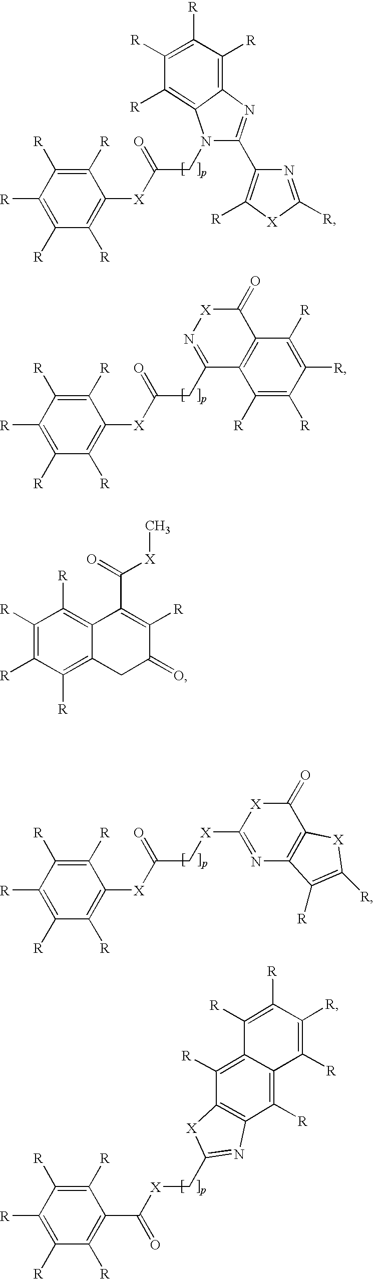

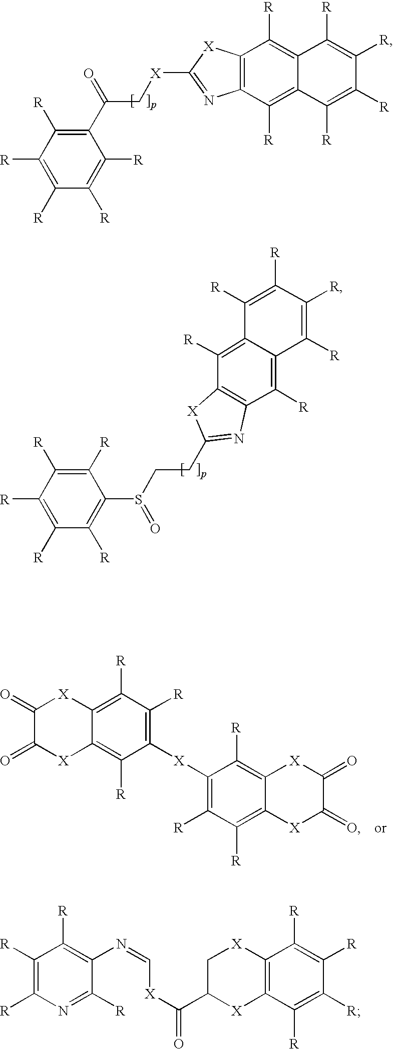

wherein, independently for each occurrence,

X is —CH₂—, —N(R^{N})—, —O—, or —S—;

p is 1-4 inclusive;

q is 0-3 inclusive;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

In certain embodiments, the present invention relates to the aforementioned compound, wherein X is —N(H)—.

In certain embodiments, the present invention relates to the aforementioned compound, wherein X is —O—

In certain embodiments, the present invention relates to the aforementioned compound, wherein X is —CH₂—.

In certain embodiments, the present invention relates to the aforementioned compound, wherein p is 1.

In certain embodiments, the present invention relates to the aforementioned compound, wherein p is 2.

In certain embodiments, the present invention relates to the aforementioned compound, wherein q is 0.

In certain embodiments, the present invention relates to the aforementioned compound, wherein q is 1.

In certain embodiments, the present invention relates to the aforementioned compound, wherein R is hydrogen, halogen, hydroxyl, alkoxy, amino, or amido.

In certain embodiments, the present invention relates to the aforementioned compound, wherein said compound is selected from the group consisting of

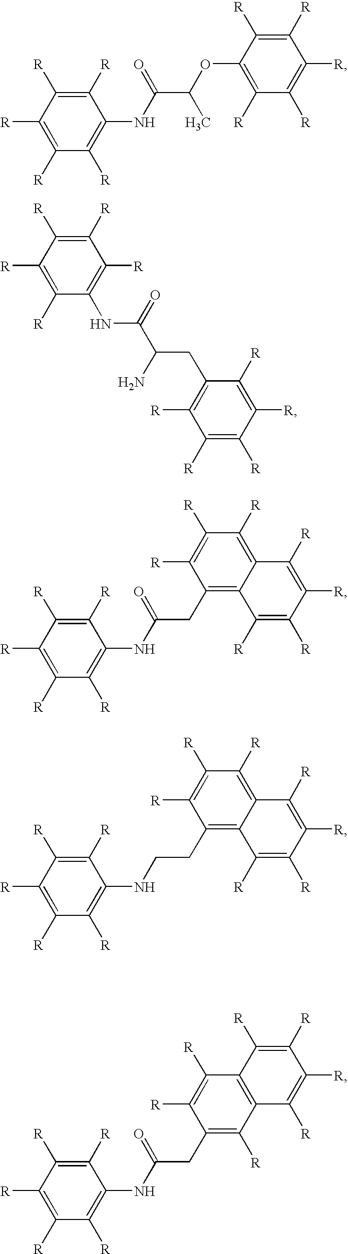

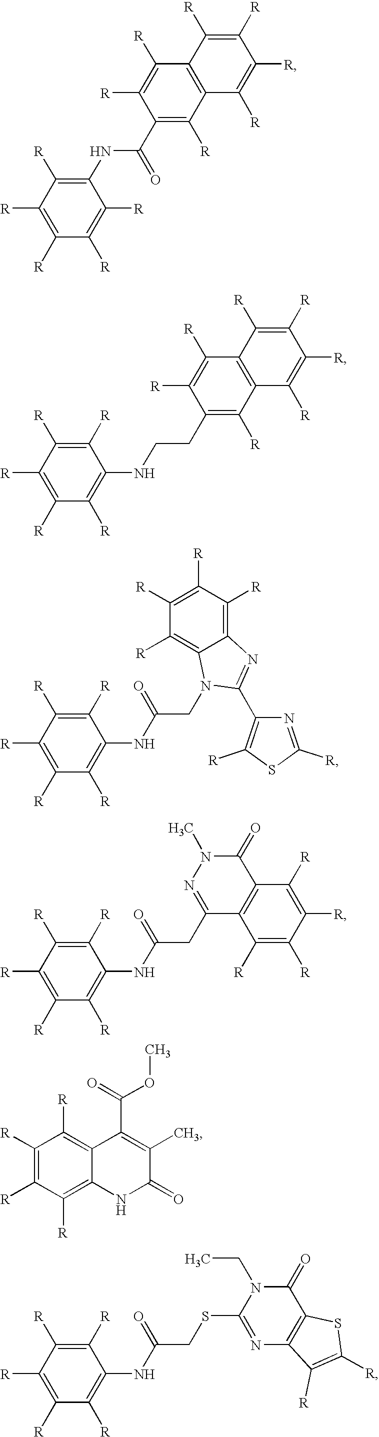

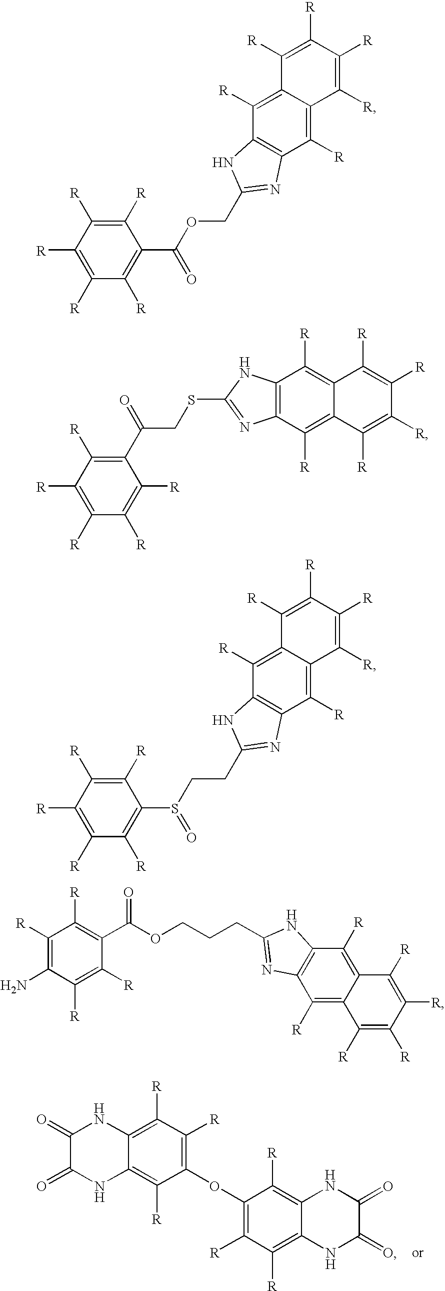

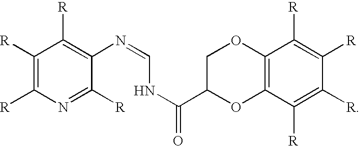

In certain embodiments, the present invention relates to the aforementioned compound, wherein R is hydrogen, halogen, hydroxyl, alkoxy, nitro, amino, or amido.

In certain embodiments, the present invention relates to the aforementioned compound, provided that the compound is not

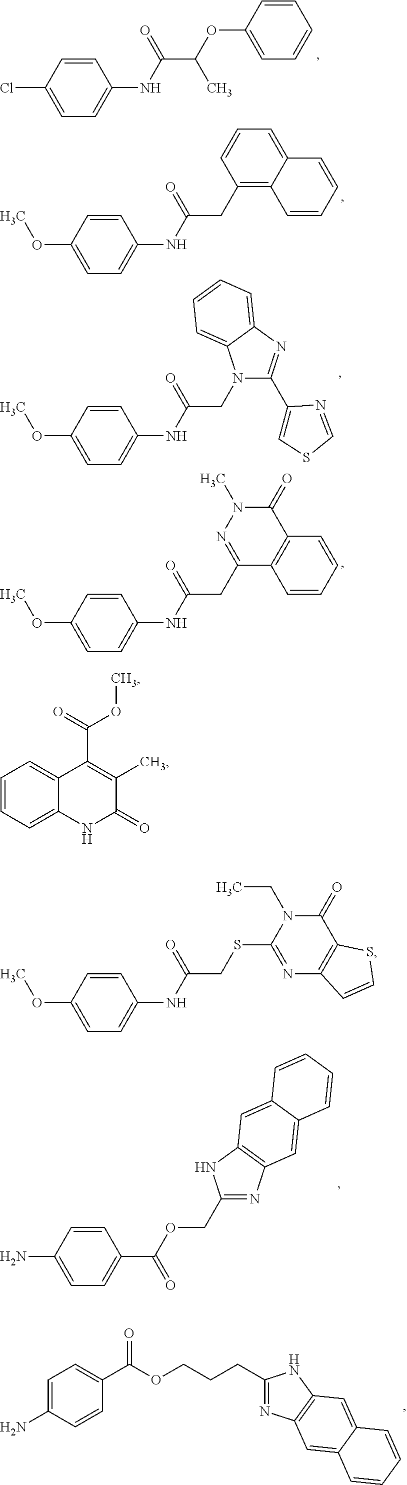

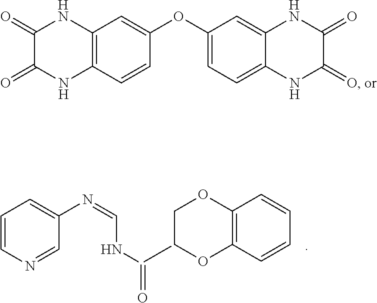

Yet another aspect of the invention relates to a compound, or a pharmaceutically acceptable salt, derivative or prodrug thereof, selected from the group consisting of

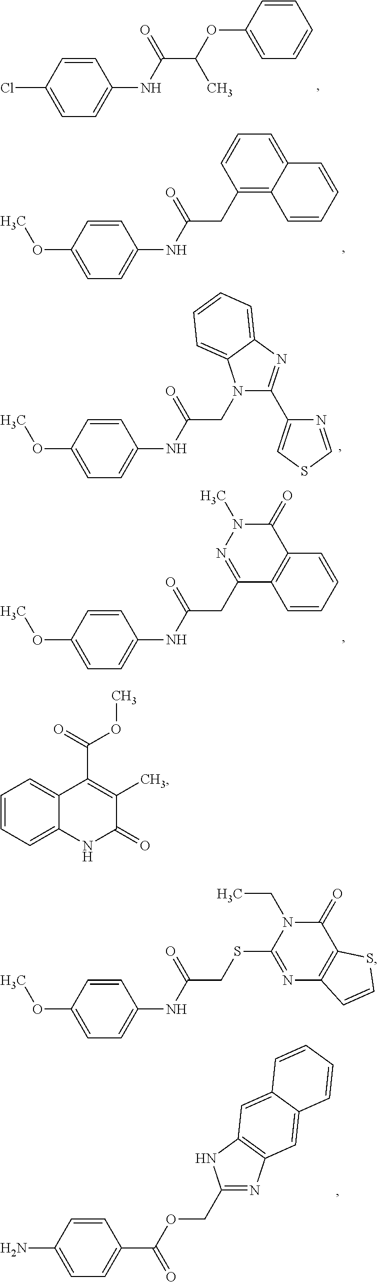

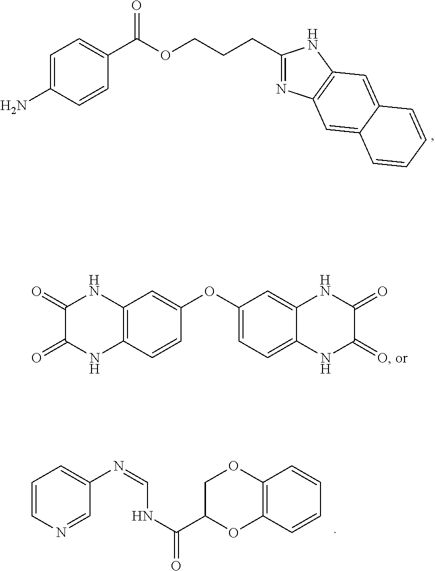

Yet another aspect of the invention relates to a compound, or a pharmaceutically acceptable salt, derivative or prodrug thereof, selected from the group consisting of

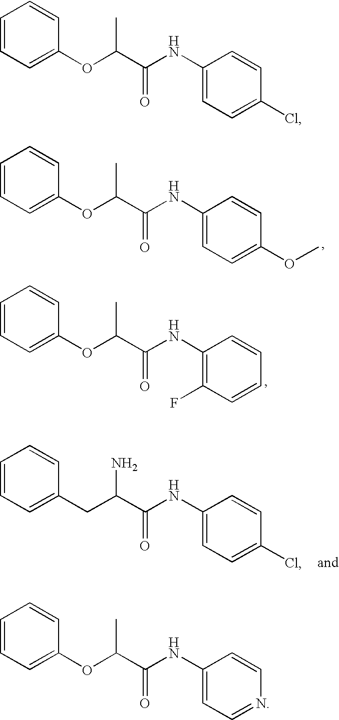

Yet another aspect of the invention relates to a compound, or a pharmaceutically acceptable salt, derivative or prodrug thereof, selected from the group consisting of

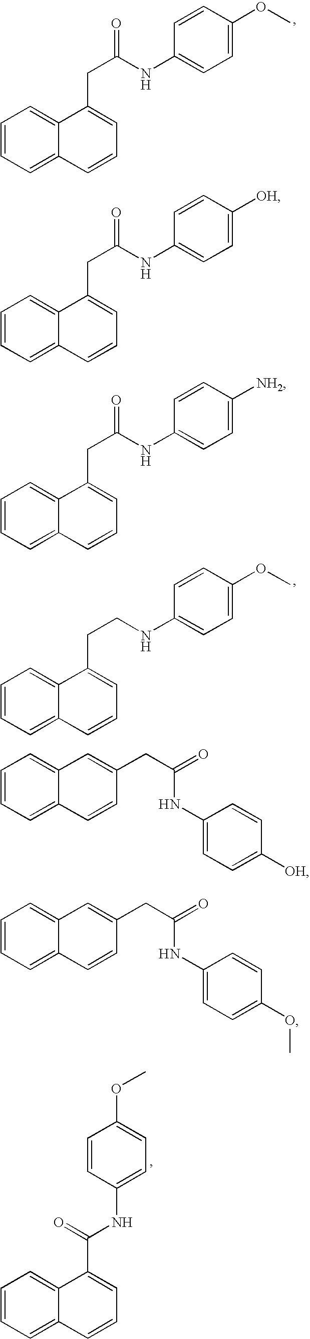

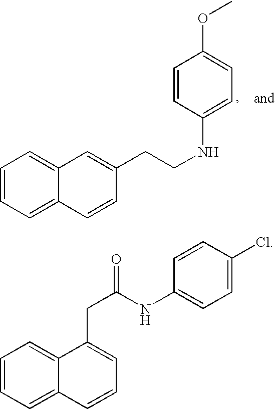

Yet another aspect of the invention relates to a compound, or a pharmaceutically acceptable salt, derivative or prodrug thereof, selected from the group consisting of

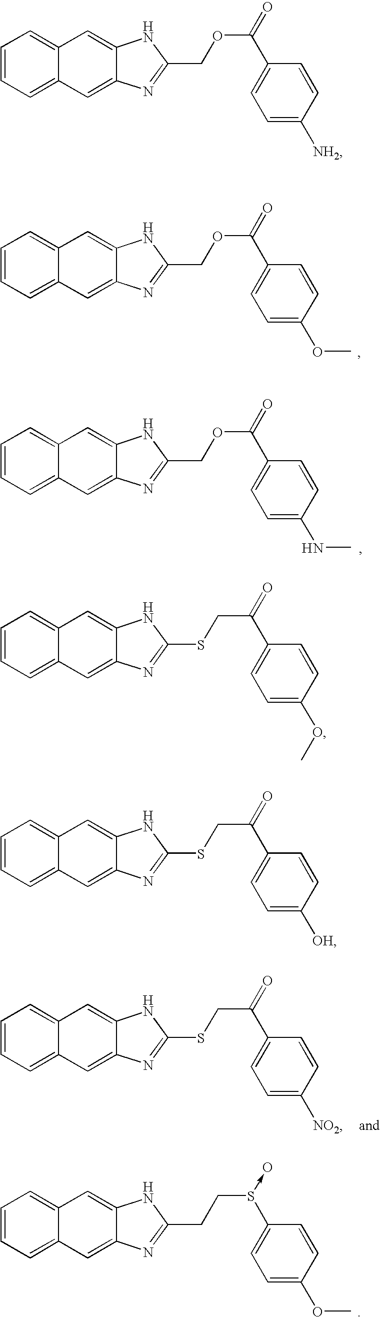

When stereochemistry is not specifically indicated, the compounds of this invention may contain one or more asymmetric carbon atoms and thus may occur as racemates and racemic mixtures, single enantiomers, diastereomeric mixtures and individual diastereomers. All such isomeric forms of these compounds are expressly included in the present invention, unless otherwise indicated. Each stereogenic carbon may be of the R or S configuration.

In addition, the compounds of this invention described above may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutical Compositions of the Invention. The compounds of this invention are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of the compounds of the invention.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium), alkaline earth metal (e.g., magnesium), and ammonium salts. This invention also envisions the quaternization of any basic nitrogen-containing groups of the compounds disclosed herein. Water or oil-soluble or dispersible products may be obtained by such quaternization.

Pharmaceutical compositions of this invention comprise a compound of the invention or a pharmaceutically acceptable salt thereof; an additional agent selected from an immunosuppressant, an anti-cancer agent, an anti-viral agent, antiinflammatory agent, antifungal agent, antibiotic, or an anti-vascular hyperproliferation compound; and any pharmaceutically acceptable carrier, adjuvant or vehicle. Alternate compositions of this invention comprise an inventive compound or a pharmaceutically acceptable salt thereof; and a pharmaceutically acceptable carrier, adjuvant or vehicle. Such composition may optionally comprise an additional agent selected from an immunosuppressant, an anti-cancer agent, an anti-viral agent, antiinflammatory agent, antifungal agent, antibiotic, or an anti-vascular hyperproliferation compound.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d.alpha.-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the inventive compounds.

The pharmaceutical compositions of this invention may be administered orally, parenterally, by inhalation spray, topically, rectally, nasally, buccally, vaginally or via an implanted reservoir. We prefer oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as those described in Pharmacopeia Helvetica, Ph. Helv., or a similar alcohol, or carboxymethyl celluose or similar dispersing agents which are commonly used in the formulation of pharmaceutically acceptable dosage forms such as emulsions and or suspensions Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxy-ethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.01 and about 100 mg/kg body weight per day, preferably between about 0.5 and about 75 mg/kg body weight per day of the IMPDH inhibitory compounds described herein are useful in a monotherapy and/or in combination therapy for the prevention and treatment of IMPDH mediated disease or infection. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

When the compositions of this invention comprise a combination of an IMPDH inhibitor of the invention and one or more additional therapeutic or prophylactic agents, both the IMPDH inhibitor and the additional agent should be present at dosage levels of between about 10 to 100%, and more preferably between about 10 to 80% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the infection, the patient's disposition to the infection and the judgment of the treating physician.

Pharmaceutical Compositions of the Invention. One aspect of the present invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

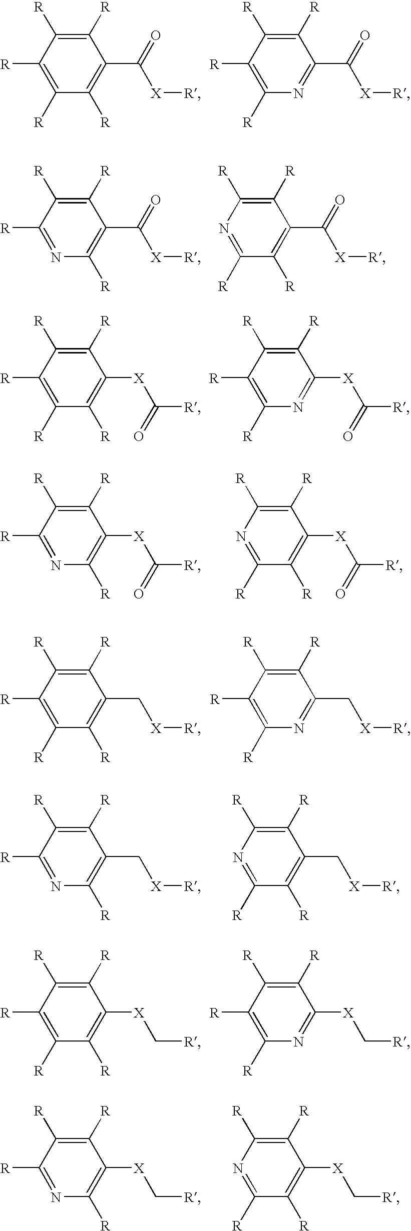

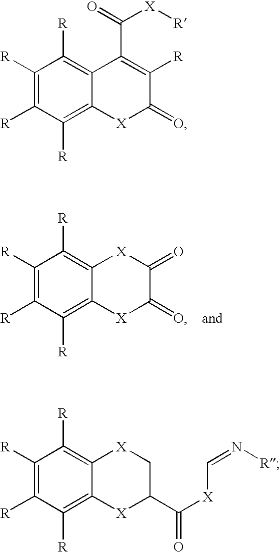

wherein, independently for each occurrence,

X is —CH₂—, —N(R^{N})—, —O—, —S(═O)₂—, —S(═O)—, or —S—;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C};

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano;

R′ is alkyl, heteroalkyl, cycloalkyl, alkcycloalkyl, alkoxycycloalkyl, alkaminocycloalkyl, alkthiocycloalkyl, heterocycloalkyl, alkylheterocycloalkyl, alkoxyheterocycloalkyl, alkaminoheterocycloalkyl, alkthioheterocycloalkyl, aryl, alkylaryl, alkoxyaryl, alkaminoaryl, alkthioaryl, heteroaryl, alkylheteroaryl, alkoxyheteroaryl, alkaminoheteroaryl, or alkthioheteroaryl,

R″ is selected from the group consisting of aryl or heteroaryl; and

any two adjacent R, taken together with the atoms to which they are directly bound, may form an optionally substituted, 5-membered or 6-membered, saturated or unsaturated, carbocyclic or heterocyclic, ring.

In certain embodiments, the present invention relates to the aforementioned pharmaceutical composition, wherein X is —O—.

In certain embodiments, the present invention relates to the aforementioned pharmaceutical composition, wherein X is —NH—

In certain embodiments, the present invention relates to the aforementioned pharmaceutical composition, wherein X is —CH₂—.

In certain embodiments, the present invention relates to the aforementioned pharmaceutical composition, wherein X is —S(═O)—.

In certain embodiments, the present invention relates to the aforementioned pharmaceutical composition, wherein R is hydrogen, halogen, alkyl, hydroxyl, alkoxy, amino, nitro, sulfhydryl, imino, amido, alkylthio, sulfonyl, sulfonamido, trifluoromethyl, or cyano.

In certain embodiments, the present invention relates to the aforementioned pharmaceutical composition, where in R′ is alkyl, heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned pharmaceutical composition, wherein R″ is monocyclic.

In certain embodiments, the present invention relates to the aforementioned pharmaceutical composition, wherein said compound is selected from the group consisting of

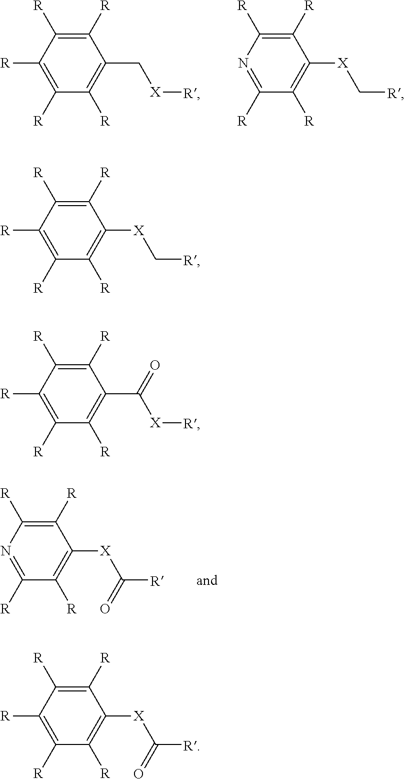

In certain embodiments, the present invention relates to the aforementioned pharmaceutical composition, wherein X is —O—.

In certain embodiments, the present invention relates to the aforementioned pharmaceutical composition, wherein X is —NH—

In certain embodiments, the present invention relates to the aforementioned pharmaceutical composition, wherein X is —CH₂—.

In certain embodiments, the present invention relates to the aforementioned pharmaceutical composition, wherein X is —S(═O)—.

In certain embodiments, the present invention relates to the aforementioned pharmaceutical composition, wherein R is hydrogen, halogen, hydroxyl, alkoxy, amino, or amido.

In certain embodiments, the present invention relates to the aforementioned pharmaceutical composition, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned pharmaceutical composition, wherein R′ is monocyclic, bicyclic or tricyclic.

In certain embodiments, the present invention relates to the aforementioned pharmaceutical composition, wherein R′ is

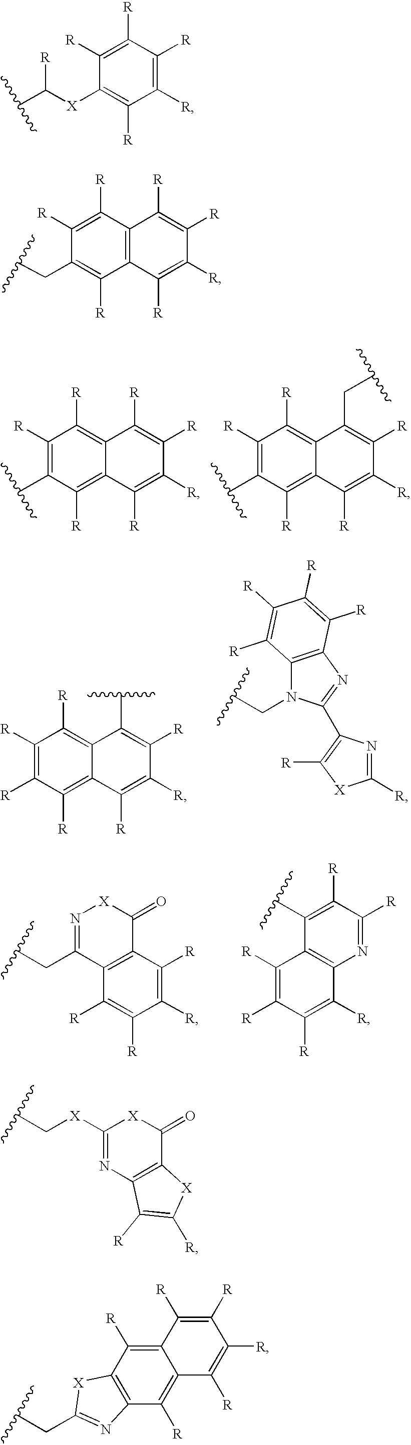

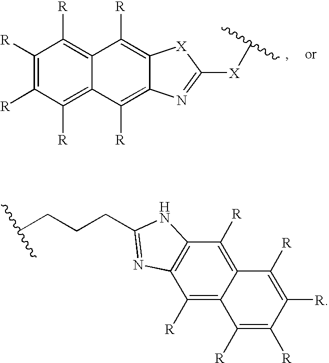

In certain embodiments, the present invention relates to the aforementioned pharmaceutical composition, wherein said compound is selected from the group consisting of

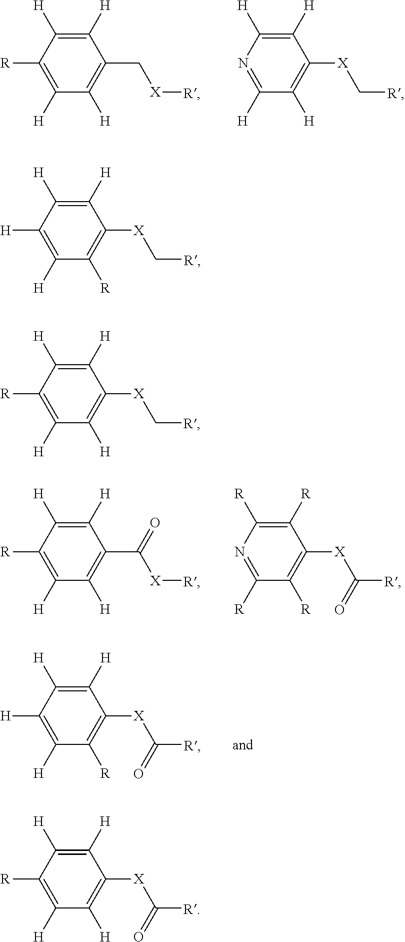

In certain embodiments, the present invention relates to the aforementioned pharmaceutical composition, wherein X is —O—.

In certain embodiments, the present invention relates to the aforementioned pharmaceutical composition, wherein X is —NH—

In certain embodiments, the present invention relates to the aforementioned pharmaceutical composition, wherein X is —CH₂—.

In certain embodiments, the present invention relates to the aforementioned pharmaceutical composition, wherein X is —S(═O)—.

In certain embodiments, the present invention relates to the aforementioned pharmaceutical composition, wherein R is hydrogen, halogen, hydroxyl, alkoxy, nitro, amino, or amido.

In certain embodiments, the present invention relates to the aforementioned pharmaceutical composition, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned pharmaceutical composition, wherein R′ is monocyclic, bicyclic or tricyclic.

In certain embodiments, the present invention relates to the aforementioned pharmaceutical composition, wherein R′ is

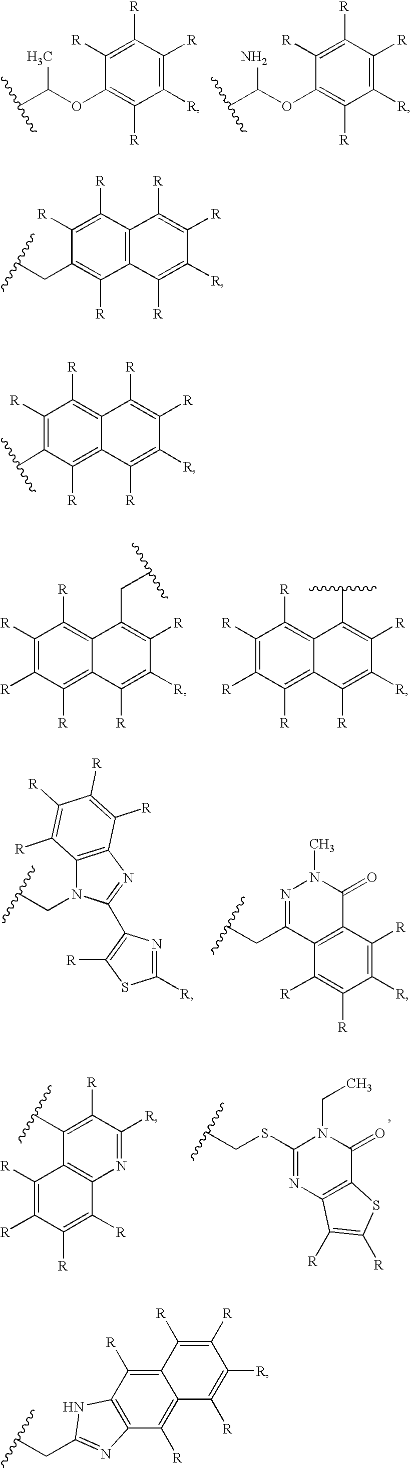

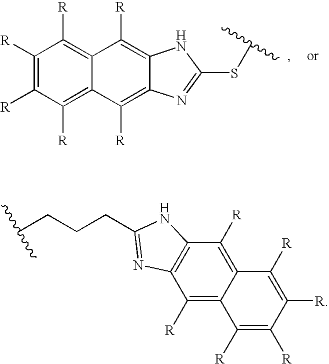

In certain embodiments, the present invention relates to the aforementioned pharmaceutical composition, wherein said compound is selected from the group consisting of

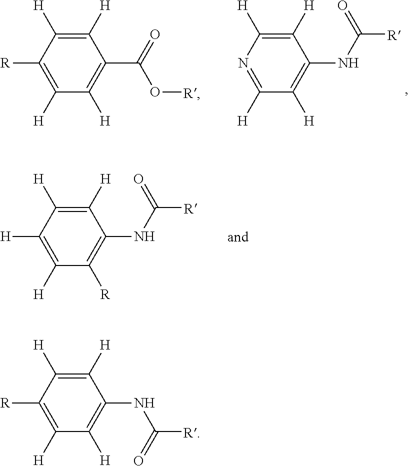

In certain embodiments, the present invention relates to the aforementioned pharmaceutical composition, wherein R is —H, —Cl, —F, —OH, —NH(CH₃), —NO₂, —OCH₃, or —NH₂.

In certain embodiments, the present invention relates to the aforementioned pharmaceutical composition, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned pharmaceutical composition, wherein R′ is monocyclic, bicyclic or tricyclic.

In certain embodiments, the present invention relates to the aforementioned pharmaceutical composition, wherein R′ is

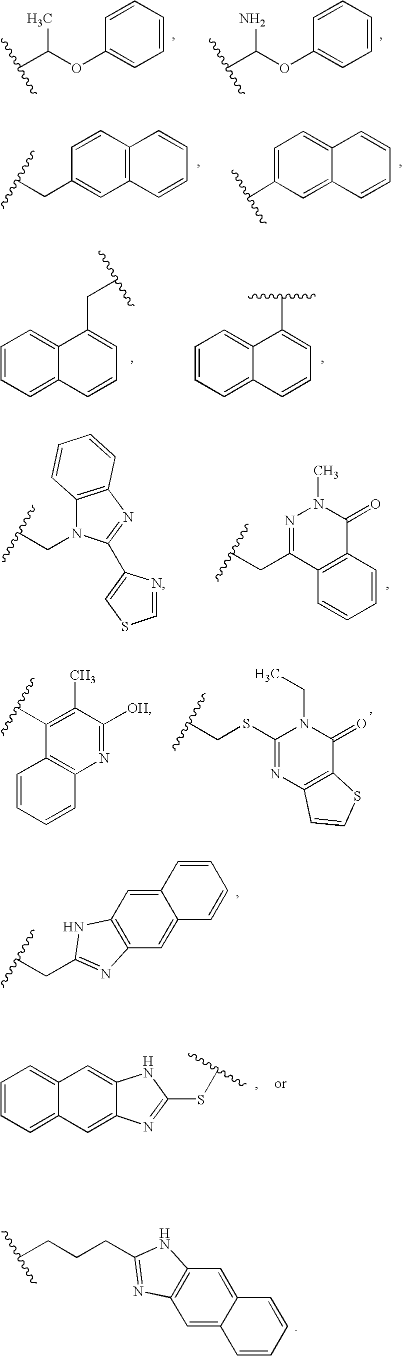

In certain embodiments, the present invention relates to the aforementioned pharmaceutical composition, wherein said compound is selected from the group consisting of

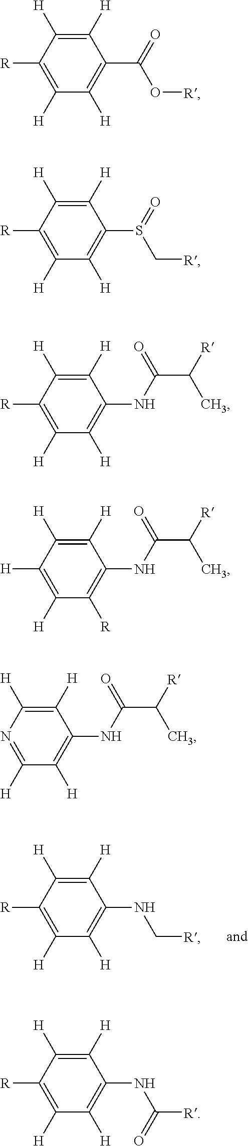

In certain embodiments, the present invention relates to the aforementioned pharmaceutical composition, wherein R is —H, —Cl, —F, —OH, —NH(CH₃), —NO₂, —OCH₃, or —NH₂.

In certain embodiments, the present invention relates to the aforementioned pharmaceutical composition, wherein R′ is heterocycloalkyl, aryl, oxyaryl, or heteroaryl.

In certain embodiments, the present invention relates to the aforementioned pharmaceutical composition, wherein R′ is monocyclic, bicyclic or tricyclic.

In certain embodiments, the present invention relates to the aforementioned pharmaceutical composition, wherein said compound is

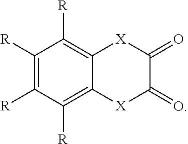

In certain embodiments, the present invention relates to the aforementioned pharmaceutical composition, wherein X is —O—, or —NH—.

In certain embodiments, the present invention relates to the aforementioned pharmaceutical composition, wherein said compound is

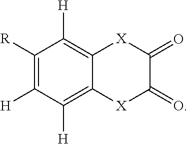

In certain embodiments, the present invention relates to the aforementioned pharmaceutical composition, wherein X is —O—, or —NH—.

In certain embodiments, the present invention relates to the aforementioned pharmaceutical composition, wherein X is —NH—.

In certain embodiments, the present invention relates to the aforementioned pharmaceutical composition, wherein R is hydroxyl, alkoxy, aryloxy, heteroaryloxy, amino, alkylamino, arylamino, or heteroarylamino.

In certain embodiments, the present invention relates to the aforementioned pharmaceutical composition, wherein R is

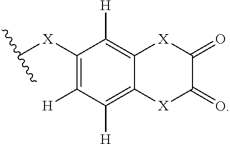

In certain embodiments, the present invention relates to the aforementioned pharmaceutical composition, wherein X is —O—, or —NH—.

In certain embodiments, the present invention relates to the aforementioned pharmaceutical composition, wherein R is

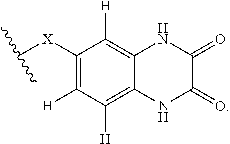

In certain embodiments, the present invention relates to the aforementioned pharmaceutical composition, wherein X is —O—, or —NH—.

In certain embodiments, the present invention relates to the aforementioned pharmaceutical composition, wherein said compound is

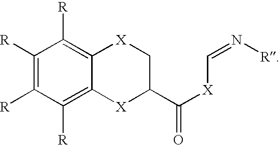

In certain embodiments, the present invention relates to the aforementioned pharmaceutical composition, wherein X is —O—, or —NH—.

In certain embodiments, the present invention relates to the aforementioned pharmaceutical composition, wherein R is hydrogen, halogen, hydroxyl, alkoxy, amino, or amido.

In certain embodiments, the present invention relates to the aforementioned pharmaceutical composition, wherein R″ is monocyclic.

In certain embodiments, the present invention relates to the aforementioned pharmaceutical composition, wherein said compound is

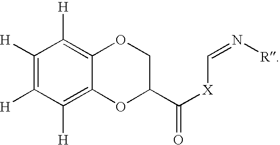

In certain embodiments, the present invention relates to the aforementioned pharmaceutical composition, wherein X is —NH—.

In certain embodiments, the present invention relates to the aforementioned pharmaceutical composition, wherein R″ is monocyclic.

In certain embodiments, the present invention relates to the aforementioned pharmaceutical composition, wherein R is phenyl, or pyridine.

In certain embodiments, the present invention relates to the aforementioned pharmaceutical composition, wherein said compound is

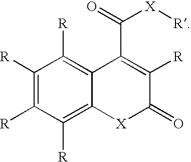

In certain embodiments, the present invention relates to the aforementioned pharmaceutical composition, wherein X is —O—, or —NH—.

In certain embodiments, the present invention relates to the aforementioned pharmaceutical composition, wherein R is hydrogen, halogen, hydroxyl, alkoxy, amino, or amido.

In certain embodiments, the present invention relates to the aforementioned pharmaceutical composition, wherein R″ is alkyl.

In certain embodiments, the present invention relates to the aforementioned pharmaceutical composition, wherein said compound is

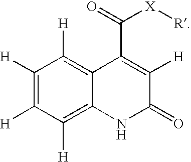

In certain embodiments, the present invention relates to the aforementioned pharmaceutical composition, wherein X is —O—, or —NH—.

In certain embodiments, the present invention relates to the aforementioned pharmaceutical composition, wherein R″ is alkyl.

In certain embodiments, the present invention relates to the aforementioned pharmaceutical composition, excluding a compound selected from the group consisting of

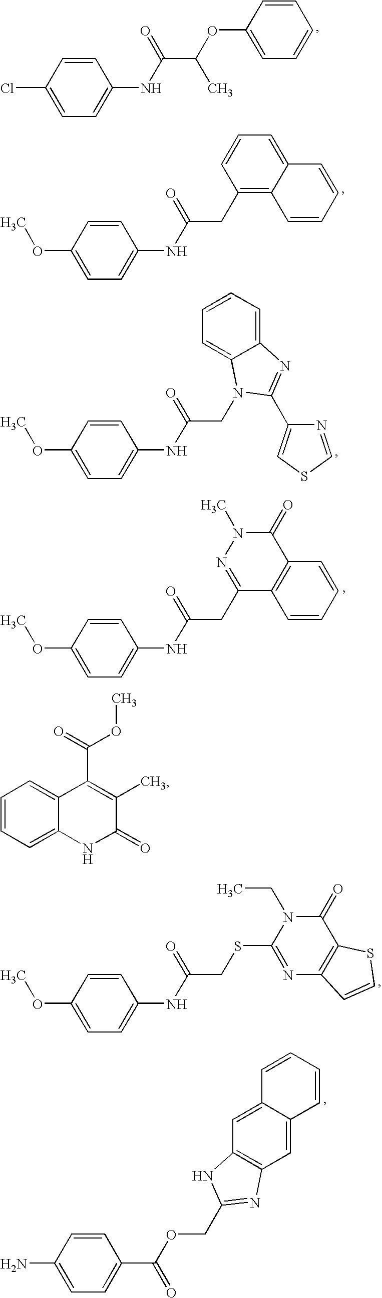

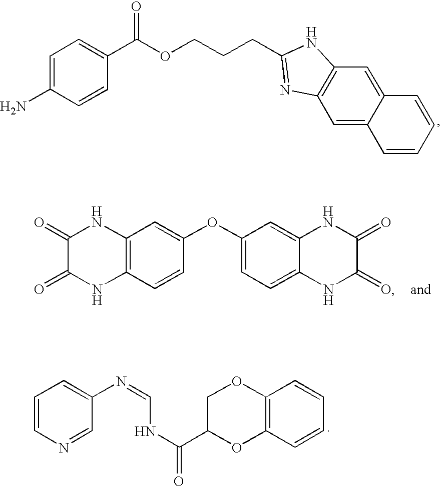

Another aspect of the present invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

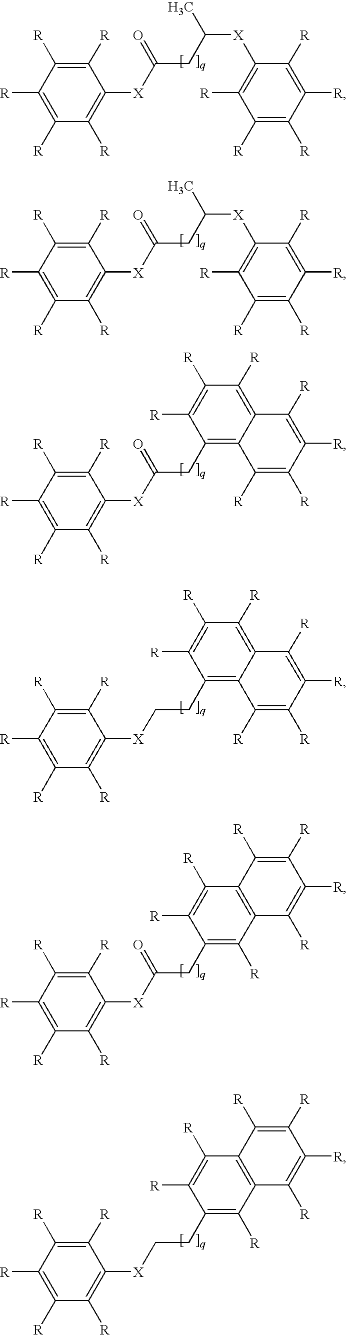

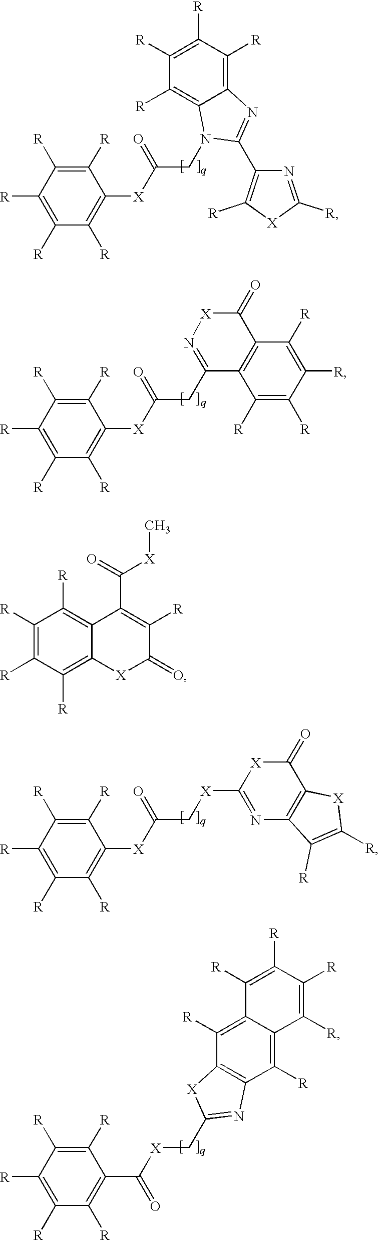

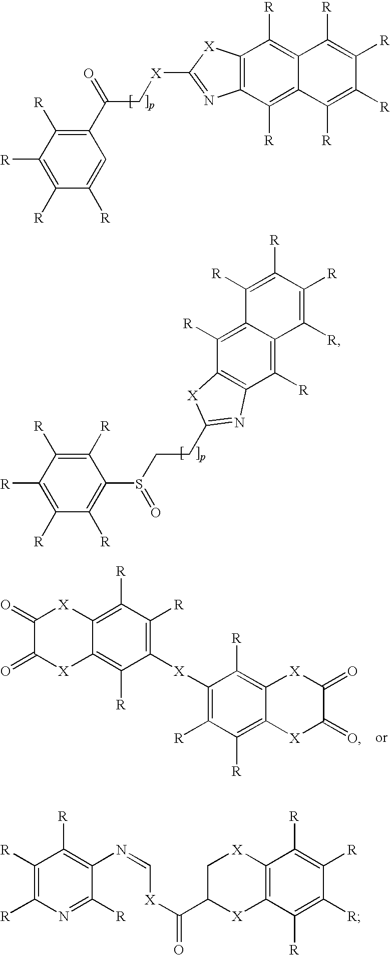

wherein, independently for each occurrence,

X is —CH₂—, —N(R^{N})—, —O—, or —S—;

p is 1-4 inclusive;

q is 0-3 inclusive;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

In certain embodiments, the present invention relates to the aforementioned pharmaceutical composition, wherein X is —N(H)—.

In certain embodiments, the present invention relates to the aforementioned pharmaceutical composition, wherein X is —O—

In certain embodiments, the present invention relates to the aforementioned pharmaceutical composition, wherein X is —CH₂—.

In certain embodiments, the present invention relates to the aforementioned pharmaceutical composition, wherein p is 1.

In certain embodiments, the present invention relates to the aforementioned pharmaceutical composition, wherein p is 2.

In certain embodiments, the present invention relates to the aforementioned pharmaceutical composition, wherein q is 0.

In certain embodiments, the present invention relates to the aforementioned pharmaceutical composition, wherein q is 1.

In certain embodiments, the present invention relates to the aforementioned pharmaceutical composition, wherein R is hydrogen, halogen, hydroxyl, alkoxy, amino, or amido.

In certain embodiments, the present invention relates to the aforementioned pharmaceutical composition, wherein said compound is selected from the group consisting of

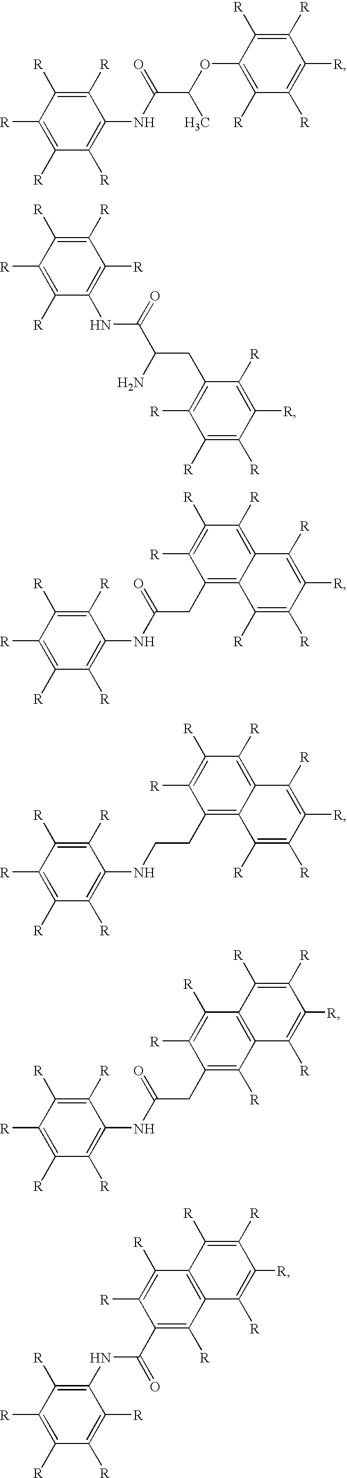

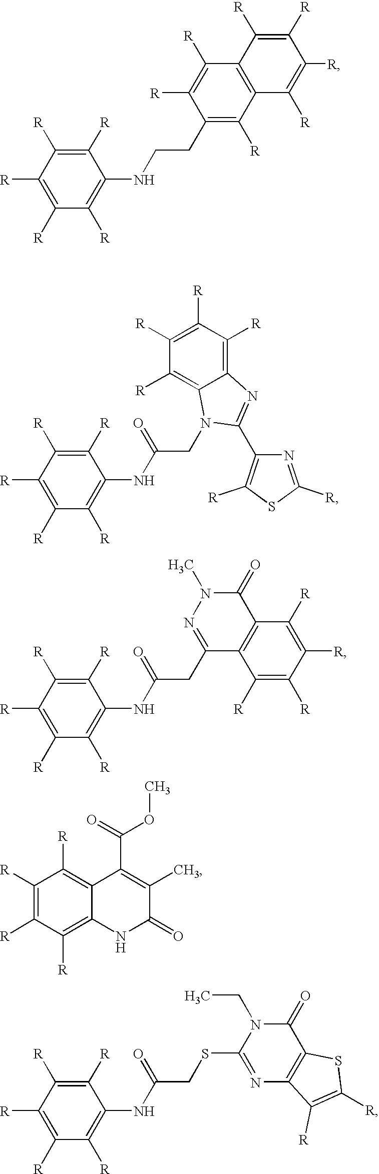

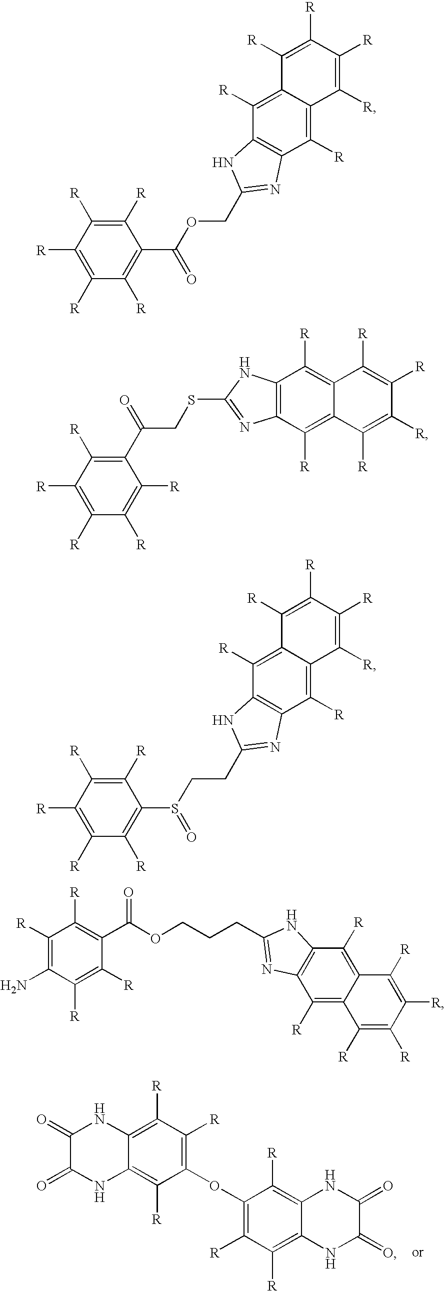

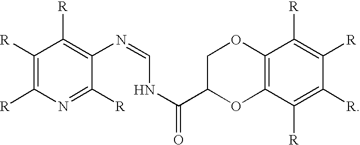

In certain embodiments, the present invention relates to the aforementioned pharmaceutical composition, wherein R is hydrogen, halogen, hydroxyl, alkoxy, nitro, amino, or amido.

In certain embodiments, the present invention relates to the aforementioned pharmaceutical composition, provided that the compound is not

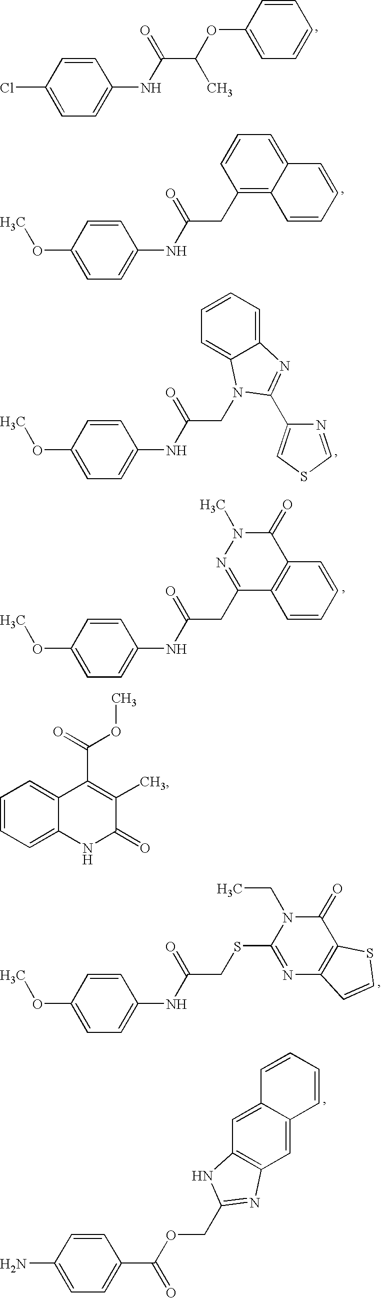

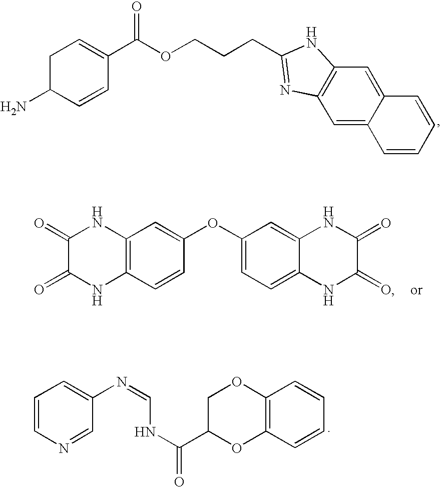

Yet another aspect of the invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

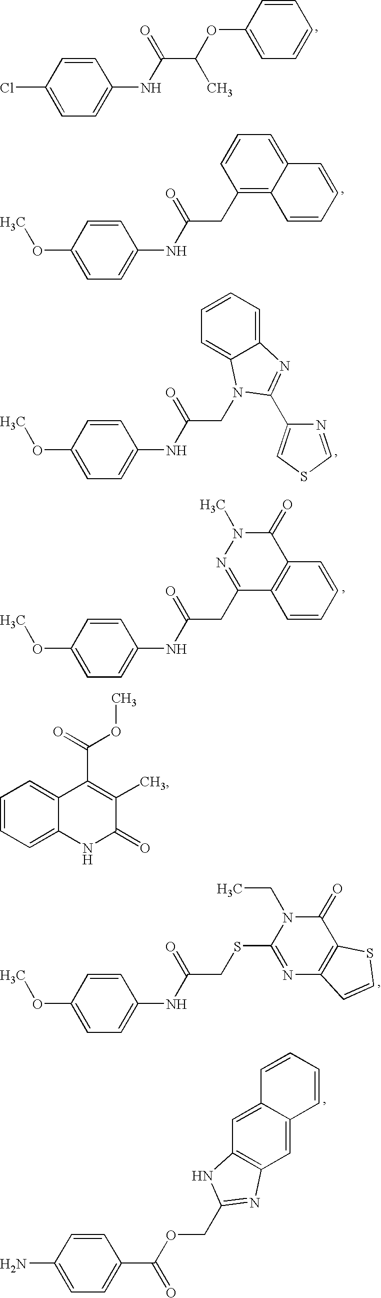

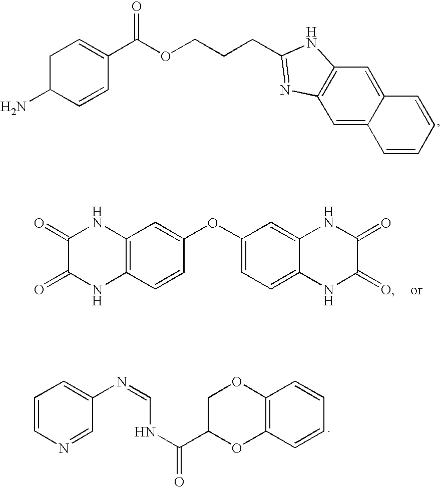

Yet another aspect of the invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

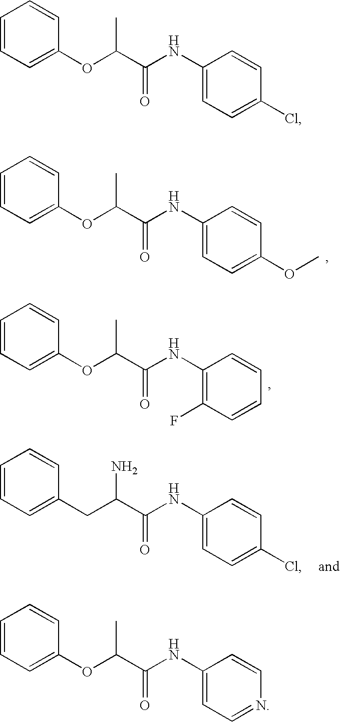

Yet another aspect of the invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

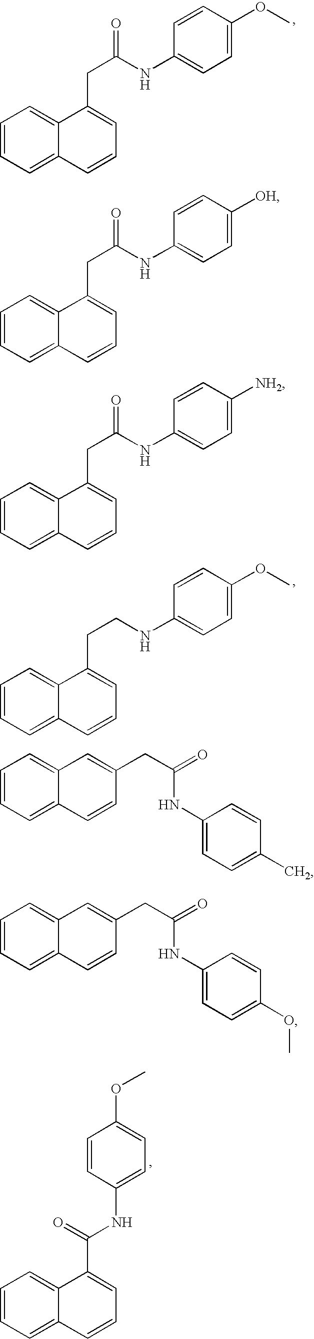

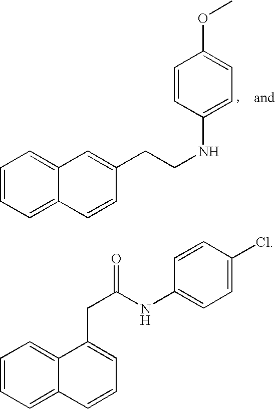

Yet another aspect of the invention relates to a pharmaceutical composition for treatment or prevention of an protozoan infection, comprising a pharmaceutically acceptable carrier, adjuvant or vehicle and at least one compound, or a pharmaceutically acceptable salt, derivative or prodrug thereof, selected from the group consisting of

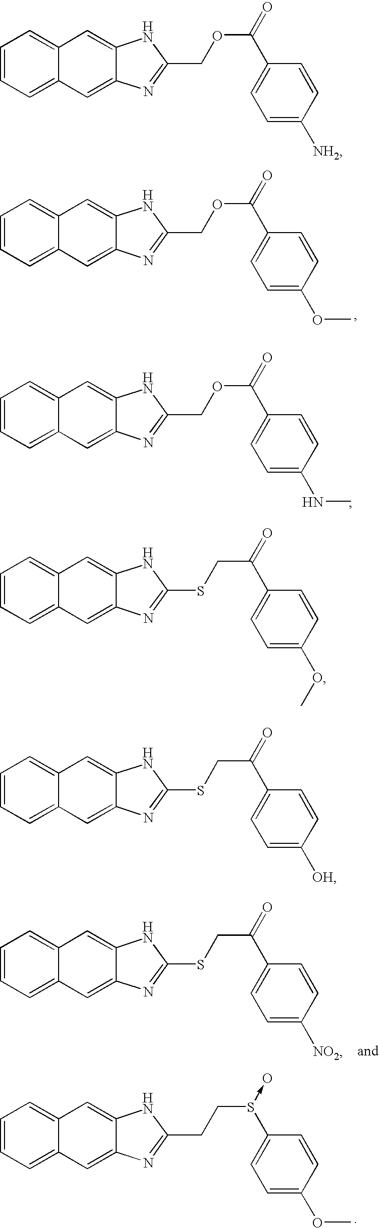

In certain embodiments, the present invention relates to the aforementioned pharmaceutical compositions, wherein said protozoan infection is caused by a protozoan selected from the group consisting of the genera Toxoplasma, Eimeria, Cryptosporidium, Plasmodium, Babesia, Theileria, Neospora, Sarcocystis, Giardia, Entamoeba, Trichomonas, Leishmania and Trypanosoma.

In certain embodiments, the present invention relates to the aforementioned pharmaceutical composition, wherein said protozoan infection is caused by Cryptosporidium parvum.

According to one embodiment, the pharmaceutical compositions of this invention may additionally comprise an antimicrobial agent, such as an antibiotic, antifungal or antiprotozoal agent. Examples of antibiotic agents include, but are not limited to, vancomycin, metronidazole, amoxicillin, ciprofloxacin, doxycycline, gentamicin and clindamycin. Examples of antifungal include, but are not limited to, terbinafine, flucytosine, fluconazole, itraconazole, ketoconazole, voriconazole, nikkomycin Z, caspofungin, micafungin (FK463), anidulafungin (LY303366), amphotericin B (AmB), and nystatin. Examples of antiprotozoal agents include, but are not limited to, eflornithine, furazolidone, melarsoprol, metronidazole, ornidazole, paromomycin sulfate, pentamidine, pyrimethamine, and tinidazole.

Another aspect of the present invention relates to a pharmaceutical compositions for treatment or prevention of an IMPDH-mediated disease, comprising a pharmaceutically acceptable carrier, adjuvant or vehicle and at least one aforementioned compound.

In certain embodiments, the pharmaceutical compositions of this invention comprise an additional immunosuppression agent. Examples of additional immunosuppression agents include, but are not limited to, cyclosporin A, FK506, rapamycin, leflunomide, deoxyspergualin, prednisone, azathioprine, mycophenolate mofetil, OKT3, ATAG, interferon and mizoribine.

In certain embodiments, the pharmaceutical compositions of this invention may additionally comprise an anti-cancer agent. Examples of anti-cancer agents include, but are not limited to, cis-platin, actinomycin D, doxorubicin, vincristine, vinblastine, etoposide, amsacrine, mitoxantrone, tenipaside, taxol, colchicine, cyclosporin A, phenothiazines, interferon and thioxantheres.

In certain embodiments, the pharmaceutical compositions of this invention may additionally comprise an anti-viral agent. Examples of anti-viral agents include, but are not limited to, cytovene, ganciclovir, trisodium phosphonoformate, Ribavirin, d4T, ddI, AZT, and acyclovir.

In certain embodiments, the pharmaceutical compositions of this invention may additionally comprise an anti-vascular hyperproliferative agent. Examples of anti-vascular hyperproliferative agents include, but are not limited to, HMG Co-A reductase inhibitors such as lovastatin, thromboxane A2 synthetase inhibitors, eicosapentanoic acid, ciprostene, trapidil, ACE inhibitors, low molecular weight heparin, mycophenolic acid, rapamycin and 5-(3′-pyridinylmethyl)benzo furan-2-carboxylate.

Selected Methods of the Invention. One aspect of the invention relates to a method of killing or inhibiting the growth of a microbe, comprising the step of contacting said microbe with a compound, or a pharmaceutically acceptable salt, derivative or prodrug thereof; wherein said compound is selected from the group consisting of

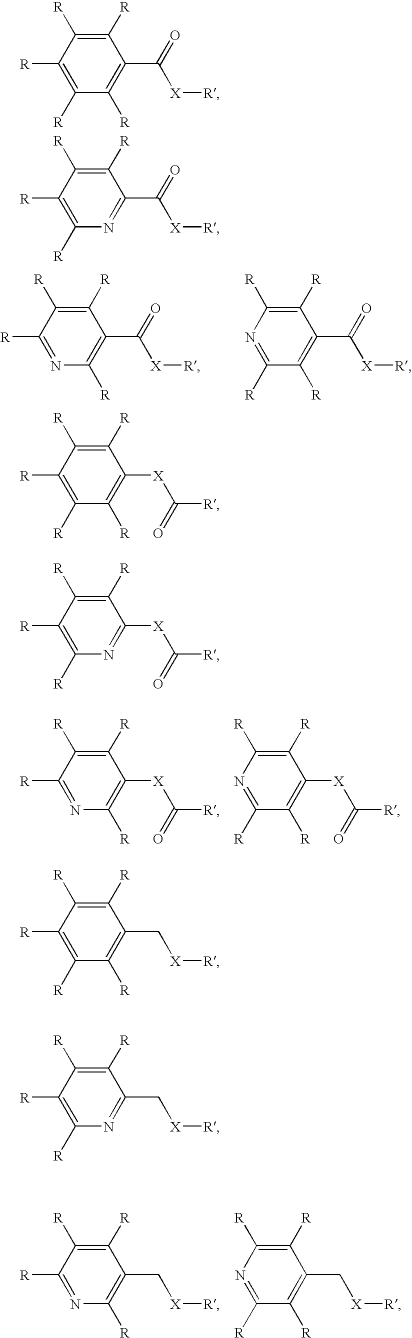

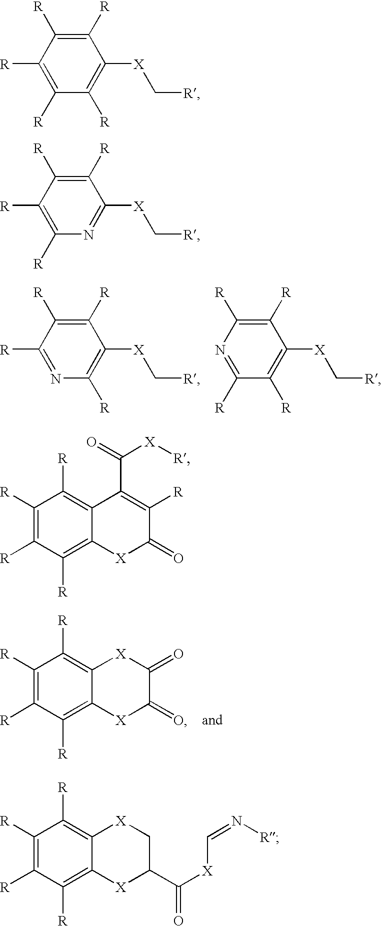

wherein, independently for each occurrence,

X is —CH₂—, —N(R^{N})—, —O—, —S(═O)₂—, —S(═O)—, or —S—;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C};

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano;

R′ is alkyl, heteroalkyl, cycloalkyl, alkcycloalkyl, alkoxycycloalkyl, alkaminocycloalkyl, alkthiocycloalkyl, heterocycloalkyl, alkylheterocycloalkyl, alkoxyheterocycloalkyl, alkaminoheterocycloalkyl, alkthioheterocycloalkyl, aryl, alkylaryl, alkoxyaryl, alkaminoaryl, alkthioaryl, heteroaryl, alkylheteroaryl, alkoxyheteroaryl, alkaminoheteroaryl, or alkthioheteroaryl,

R″ is selected from the group consisting of aryl or heteroaryl; and

any two adjacent R, taken together with the atoms to which they are directly bound, may form an optionally substituted, 5-membered or 6-membered, saturated or unsaturated, carbocyclic or heterocyclic, ring.

Another aspect of the invention relates to a method of killing or inhibiting the growth of a protozoa, comprising the step of contacting said protozoa with a compound, or a pharmaceutically acceptable salt, derivative or prodrug thereof, wherein said compound is selected from the group consisting of

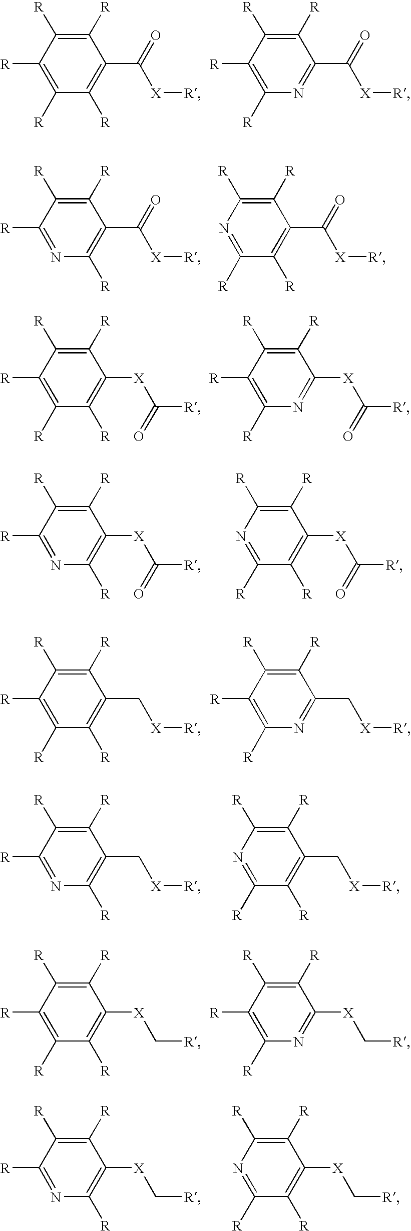

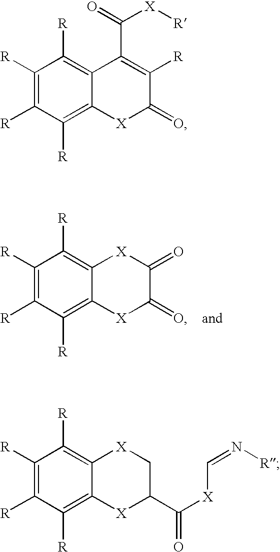

wherein, independently for each occurrence,

X is —CH₂—, —N(R^{N})—, —O—, —S(═O)₂—, —S(═O)—, or —S—;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C};

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano;

R′ is alkyl, heteroalkyl, cycloalkyl, alkcycloalkyl, alkoxycycloalkyl, alkaminocycloalkyl, alkthiocycloalkyl, heterocycloalkyl, alkylheterocycloalkyl, alkoxyheterocycloalkyl, alkaminoheterocycloalkyl, alkthioheterocycloalkyl, aryl, alkylaryl, alkoxyaryl, alkaminoaryl, alkthioaryl, heteroaryl, alkylheteroaryl, alkoxyheteroaryl, alkaminoheteroaryl, or alkthioheteroaryl,

R″ is selected from the group consisting of aryl or heteroaryl; and

any two adjacent R, taken together with the atoms to which they are directly bound, may form an optionally substituted, 5-membered or 6-membered, saturated or unsaturated, carbocyclic or heterocyclic, ring.

In certain embodiments, the present invention relates to the aforementioned method, wherein X is —O—.

In certain embodiments, the present invention relates to the aforementioned method, wherein X is —NH—

In certain embodiments, the present invention relates to the aforementioned method, wherein X is —CH₂—.

In certain embodiments, the present invention relates to the aforementioned method, wherein X is —S(═O)—.

In certain embodiments, the present invention relates to the aforementioned method, wherein R is hydrogen, halogen, alkyl, hydroxyl, alkoxy, amino, nitro, sulfhydryl, imino, amido, alkylthio, sulfonyl, sulfonamido, trifluoromethyl, or cyano.

In certain embodiments, the present invention relates to the aforementioned method, where in R′ is alkyl, heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned method, wherein R″ is monocyclic.

In certain embodiments, the present invention relates to the aforementioned method, wherein said compound is selected from the group consisting of

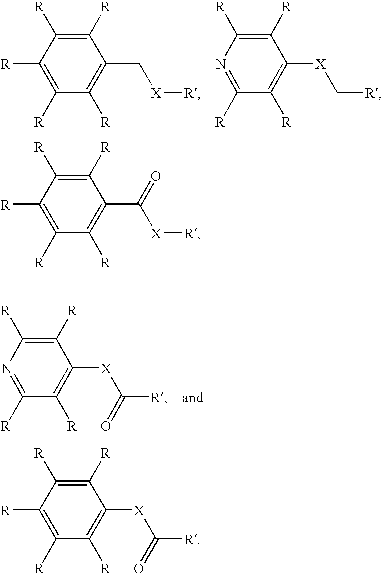

In certain embodiments, the present invention relates to the aforementioned method, wherein X is —O—.

In certain embodiments, the present invention relates to the aforementioned method, wherein X is —NH—

In certain embodiments, the present invention relates to the aforementioned method, wherein X is —CH₂—.

In certain embodiments, the present invention relates to the aforementioned method, wherein X is —S(═O)—.

In certain embodiments, the present invention relates to the aforementioned method, wherein R is hydrogen, halogen, hydroxyl, alkoxy, amino, or amido.

In certain embodiments, the present invention relates to the aforementioned method, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned method, wherein R′ is monocyclic, bicyclic or tricyclic.

In certain embodiments, the present invention relates to the aforementioned method, wherein R′ is

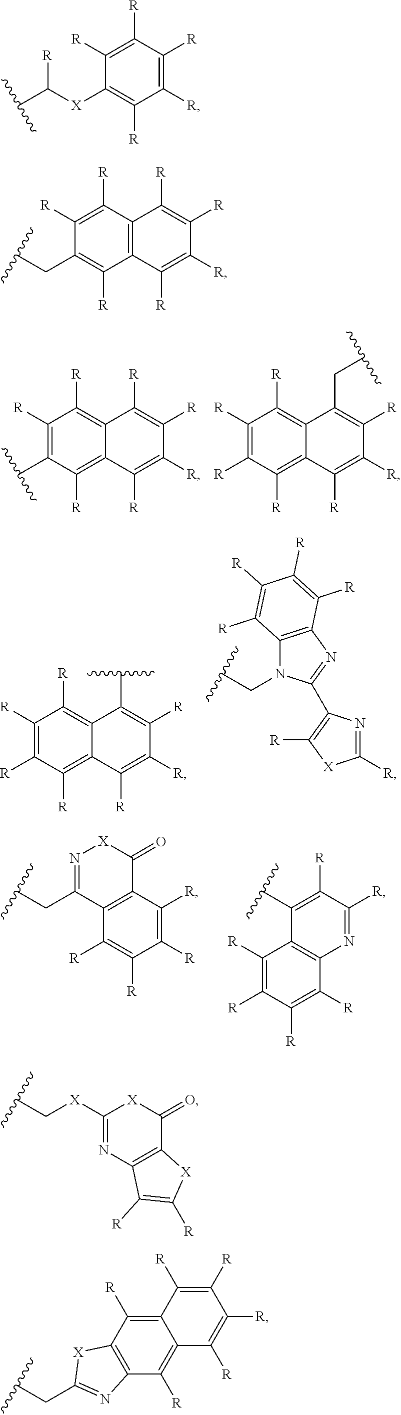

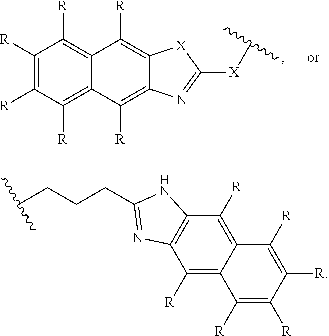

In certain embodiments, the present invention relates to the aforementioned method, wherein said compound is selected from the group consisting of

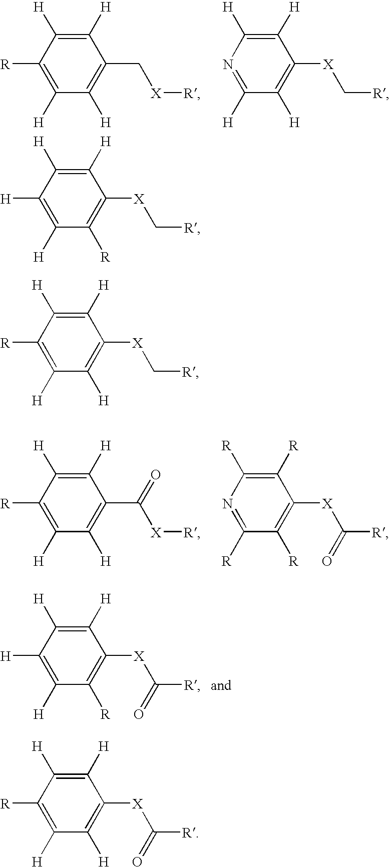

In certain embodiments, the present invention relates to the aforementioned method, wherein X is —O—.

In certain embodiments, the present invention relates to the aforementioned method, wherein X is —NH—

In certain embodiments, the present invention relates to the aforementioned method, wherein X is —CH₂—.

In certain embodiments, the present invention relates to the aforementioned method, wherein X is —S(═O)—.

In certain embodiments, the present invention relates to the aforementioned method, wherein R is hydrogen, halogen, hydroxyl, alkoxy, nitro, amino, or amido.

In certain embodiments, the present invention relates to the aforementioned method, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned method, wherein R′ is monocyclic, bicyclic or tricyclic.

In certain embodiments, the present invention relates to the aforementioned method, wherein R′ is

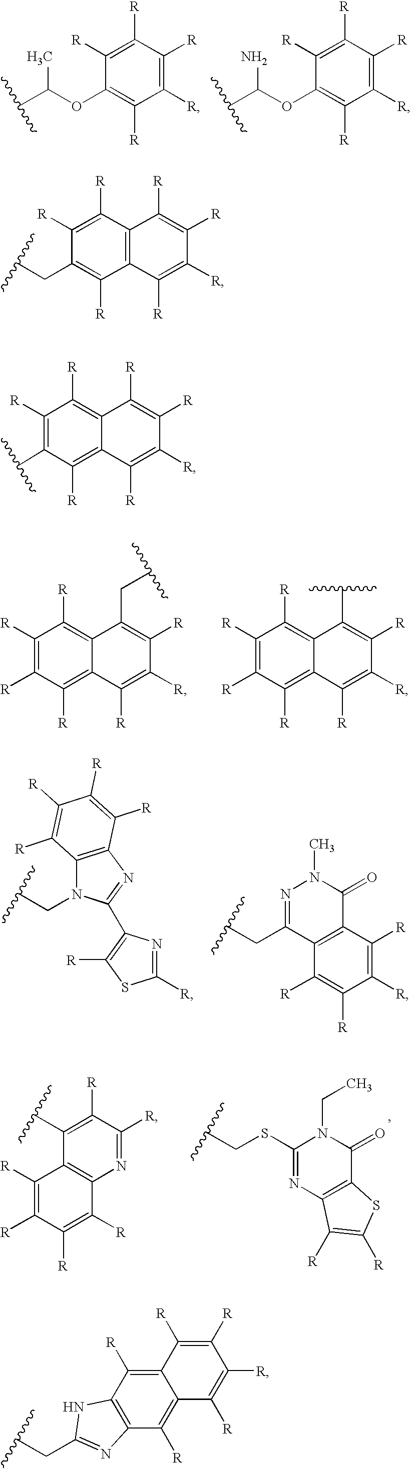

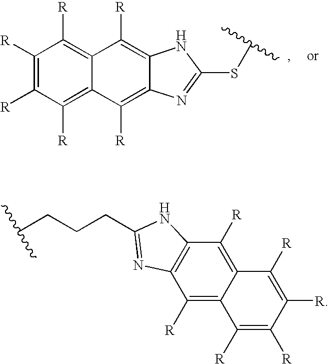

In certain embodiments, the present invention relates to the aforementioned method, wherein said compound is selected from the group consisting of

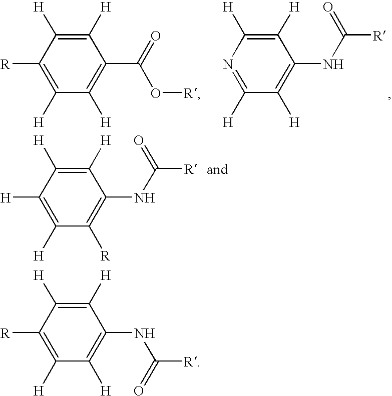

In certain embodiments, the present invention relates to the aforementioned method, wherein R is —H, —Cl, —F, —OH, —NH(CH₃), —NO₂, —OCH₃, or —NH₂.

In certain embodiments, the present invention relates to the aforementioned method, wherein R′ is heterocycloalkyl, alkylheterocycloalkyl, alkoxyheterocycloalkyl, aryl, alkylaryl, alkoxyaryl, heteroaryl, alkylheteroaryl, or alkoxyheteroaryl.

In certain embodiments, the present invention relates to the aforementioned method, wherein R′ is monocyclic, bicyclic or tricyclic.

In certain embodiments, the present invention relates to the aforementioned method, wherein R′ is

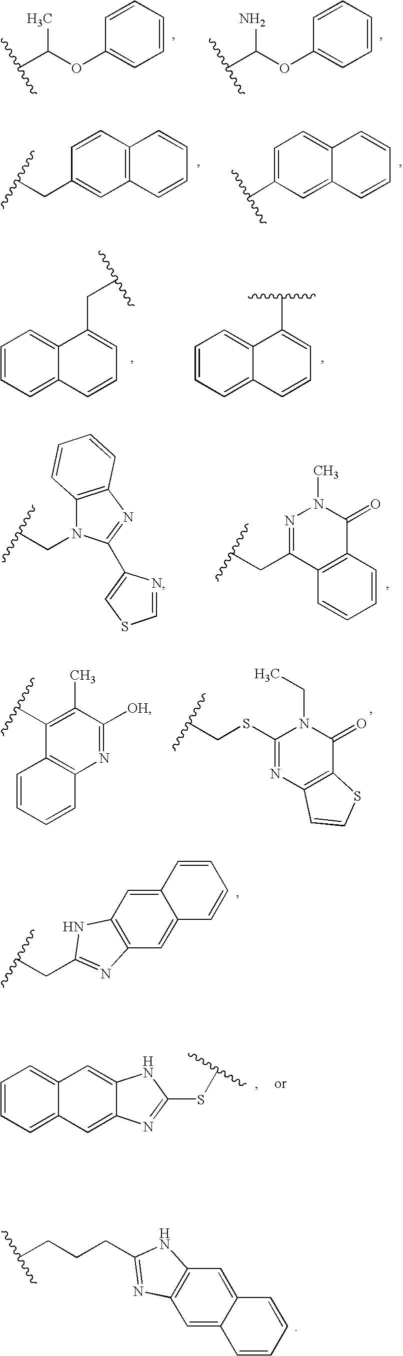

In certain embodiments, the present invention relates to the aforementioned method, wherein said compound is selected from the group consisting of

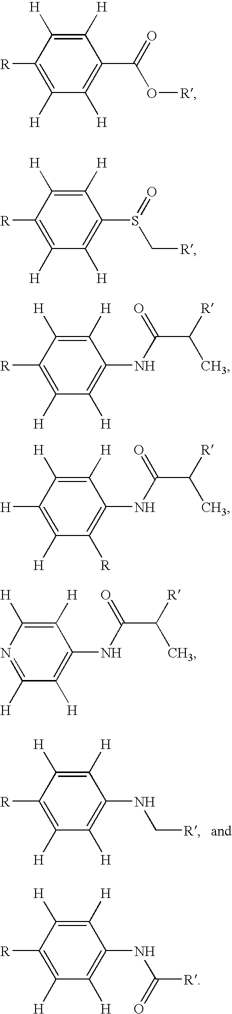

In certain embodiments, the present invention relates to the aforementioned method, wherein R is —H, —Cl, —F, —OH, —NH(CH₃), —NO₂, —OCH₃, or —NH₂.

In certain embodiments, the present invention relates to the aforementioned method, wherein R′ is heterocycloalkyl, aryl, oxyaryl, or heteroaryl.

In certain embodiments, the present invention relates to the aforementioned method, wherein R′ is monocyclic, bicyclic or tricyclic.

In certain embodiments, the present invention relates to the aforementioned method, wherein said compound is

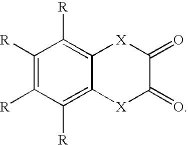

In certain embodiments, the present invention relates to the aforementioned method, wherein X is —O—, or —NH—.

In certain embodiments the present invention relates to the aforementioned method, wherein said compound is

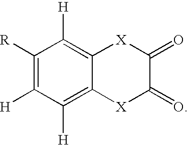

In certain embodiments, the present invention relates to the aforementioned method, wherein X is —O—, or —NH—.

In certain embodiments, the present invention relates to the aforementioned method, wherein X is —NH—.

In certain embodiments, the present invention relates to the aforementioned method, wherein R is hydroxyl, alkoxy, aryloxy, heteroaryloxy, amino, alkylamino, arylamino, or heteroarylamino.

In certain embodiments, the present invention relates to the aforementioned method, wherein R is

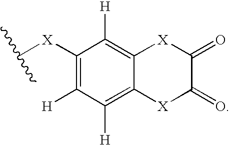

In certain embodiments, the present invention relates to the aforementioned method, wherein X is —O—, or —NH—.

In certain embodiments, the present invention relates to the aforementioned method, wherein R is

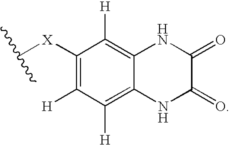

In certain embodiments, the present invention relates to the aforementioned method, wherein X is —O—, or —NH—.

In certain embodiments, the present invention relates to the aforementioned method, wherein said compound is

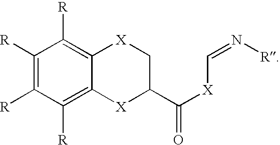

In certain embodiments, the present invention relates to the aforementioned method, wherein X is —O—, or —NH—.

In certain embodiments, the present invention relates to the aforementioned method, wherein R is hydrogen, halogen, hydroxyl, alkoxy, amino, or amido.

In certain embodiments, the present invention relates to the aforementioned method, wherein R″ is monocyclic.

In certain embodiments, the present invention relates to the aforementioned method, wherein said compound is

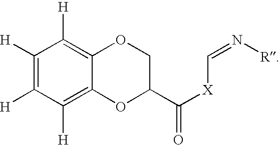

In certain embodiments, the present invention relates to the aforementioned method, wherein X is —NH—.

In certain embodiments, the present invention relates to the aforementioned method, wherein R″ is monocyclic.

In certain embodiments, the present invention relates to the aforementioned method, wherein R is phenyl, or pyridine.

In certain embodiments, the present invention relates to the aforementioned method, wherein said compound is

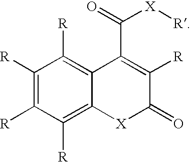

In certain embodiments, the present invention relates to the aforementioned method, wherein X is —O—, or —NH—.

In certain embodiments, the present invention relates to the aforementioned method, wherein R is hydrogen, halogen, hydroxyl, alkoxy, amino, or amido.

In certain embodiments, the present invention relates to the aforementioned method, wherein R″ is alkyl.

In certain embodiments, the present invention relates to the aforementioned method, wherein said compound is

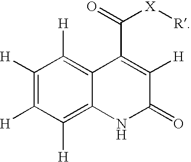

In certain embodiments, the present invention relates to the aforementioned method, wherein X is —O—, or —NH—.

In certain embodiments, the present invention relates to the aforementioned method, wherein R″ is alkyl.

In certain embodiments, the present invention relates to the aforementioned method, excluding a compound selected from the group consisting of

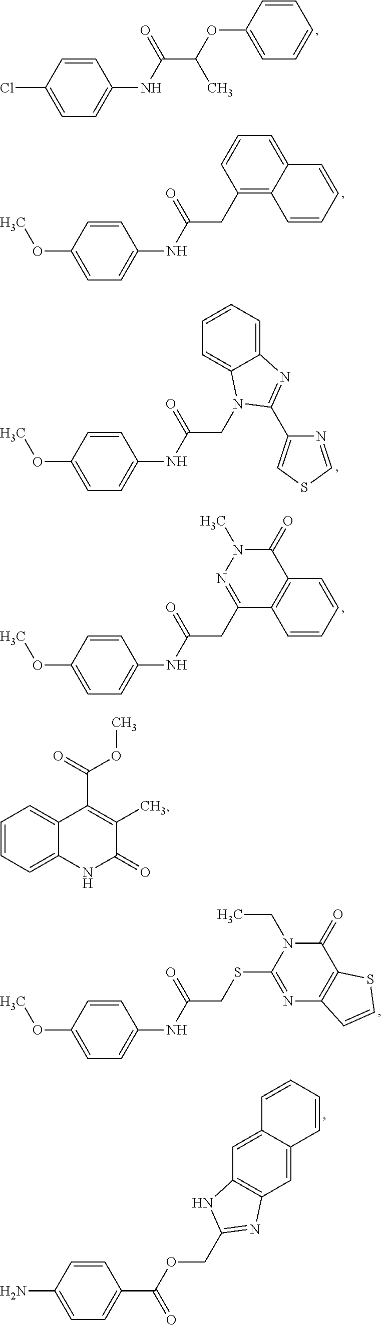

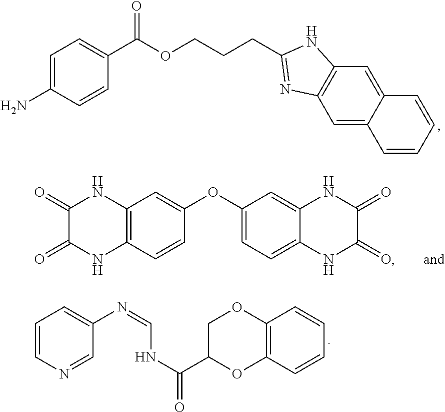

Another aspect of the invention relates to a method of killing or inhibiting the growth of a microbe comprising the step of contacting said microbe with a compound, or a pharmaceutically acceptable salt, derivative or prodrug thereof, wherein said compound is selected from the group consisting of

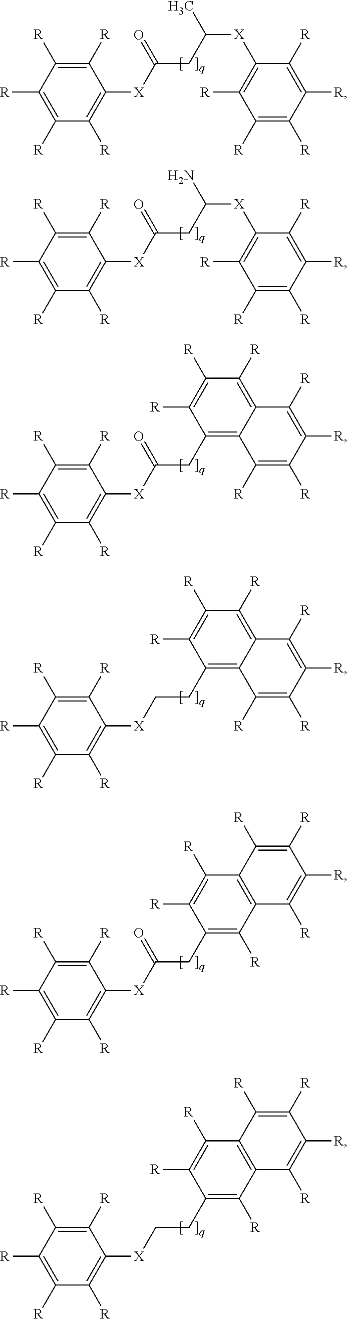

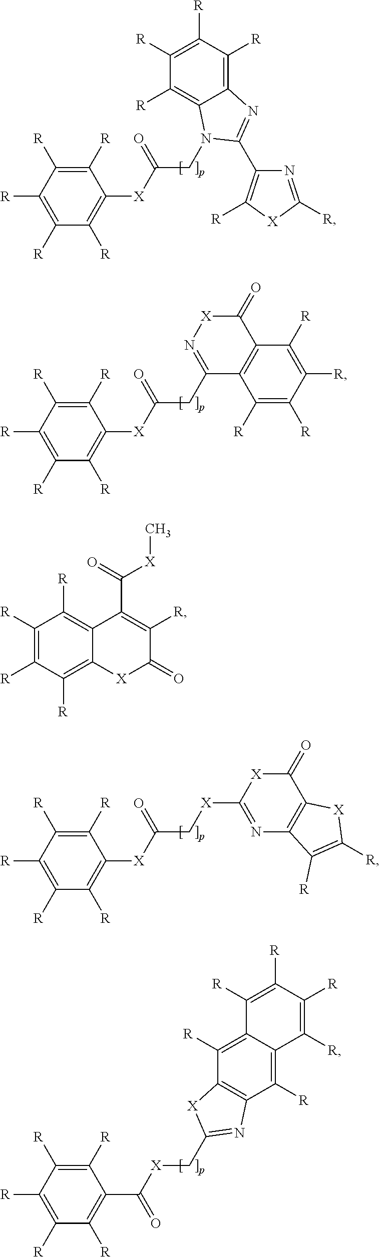

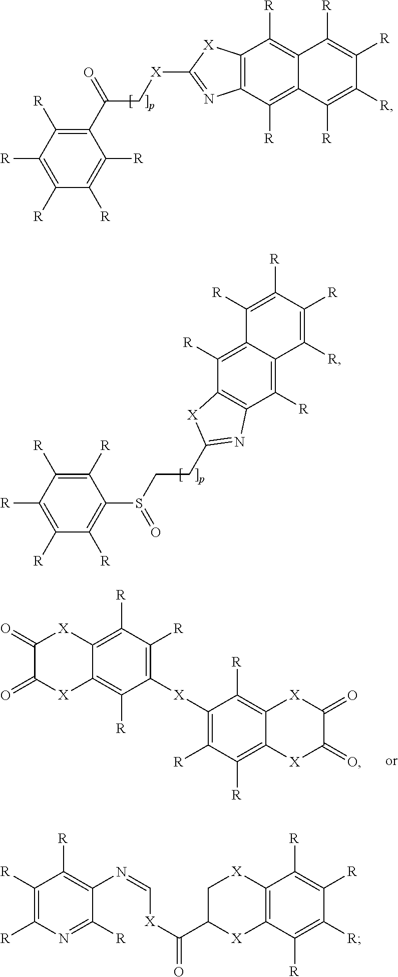

wherein, independently for each occurrence,

X is —CH₂—, —N(R^{N})—, —O—, or —S—;

p is 1-4 inclusive;

q is 0-3 inclusive;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

Another aspect of the invention relates to a method of killing or inhibiting the growth of a protozoa comprising the step of contacting said protozoa with a compound, or a pharmaceutically acceptable salt, derivative or prodrug thereof, wherein said compound is selected from the group consisting of

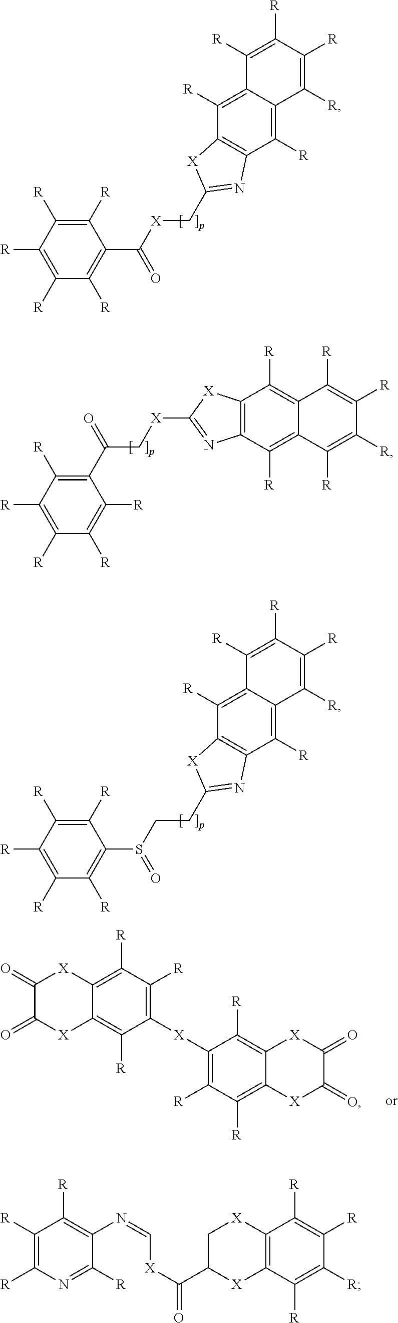

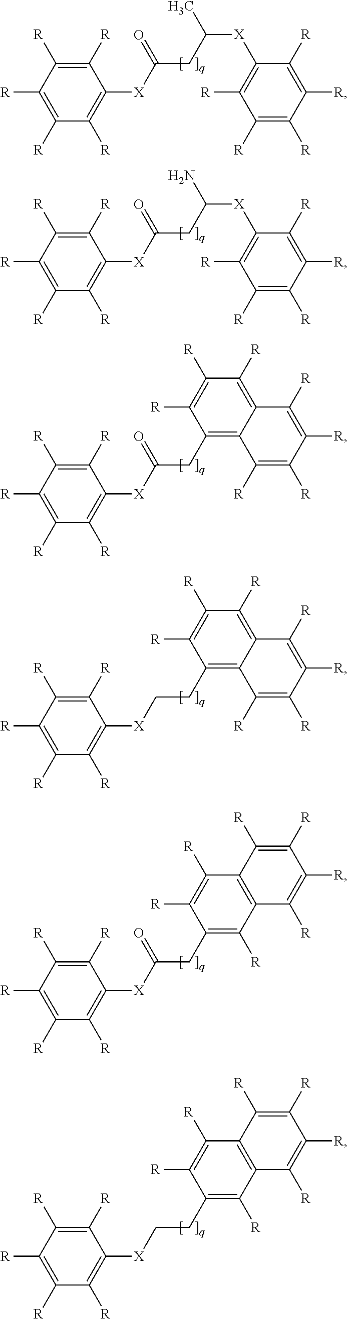

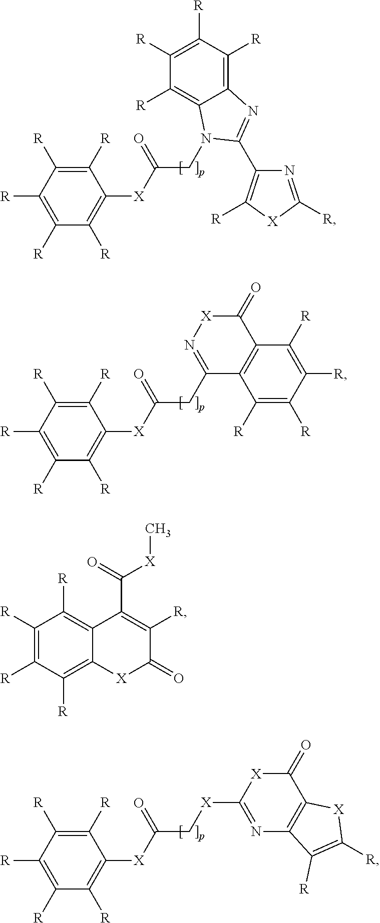

wherein, independently for each occurrence,

X is —CH₂—, —N(R^{N})—, —O—, or —S—;

p is 1-4 inclusive;

q is 0-3 inclusive;

R^{N} is hydrogen, alkyl, aralkyl, or carbonyl;

R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and

R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

In certain embodiments, the present invention relates to the aforementioned method, wherein X is —N(H)—.

In certain embodiments, the present invention relates to the aforementioned method, wherein X is —O—

In certain embodiments, the present invention relates to the aforementioned method, wherein X is —CH₂—.

In certain embodiments, the present invention relates to the aforementioned method, wherein p is 1.

In certain embodiments, the present invention relates to the aforementioned method, wherein p is 2.

In certain embodiments, the present invention relates to the aforementioned method, wherein q is 0.

In certain embodiments, the present invention relates to the aforementioned method, wherein q is 1.

In certain embodiments, the present invention relates to the aforementioned method, wherein R is hydrogen, halogen, hydroxyl, alkoxy, amino, or amido.

In certain embodiments, the present invention relates to the aforementioned method, wherein said compound is selected from the group consisting of

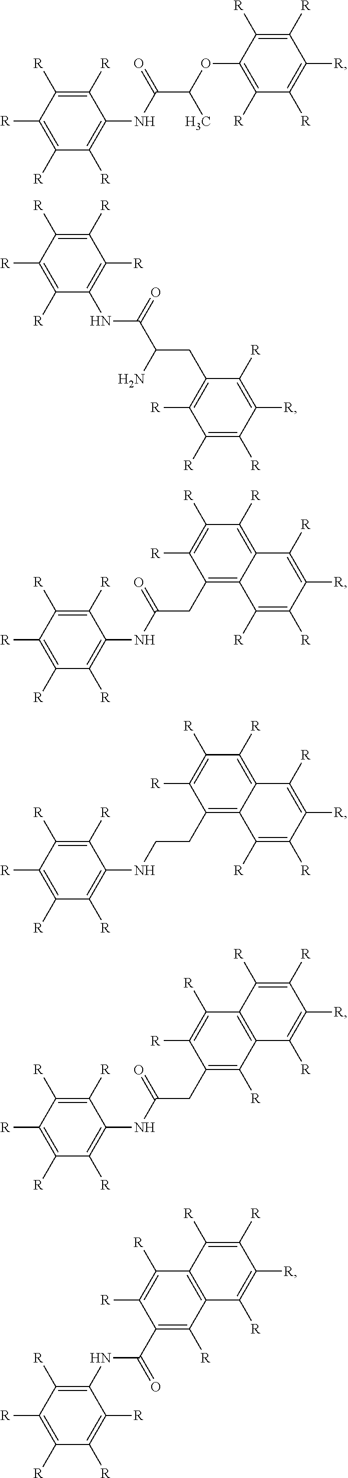

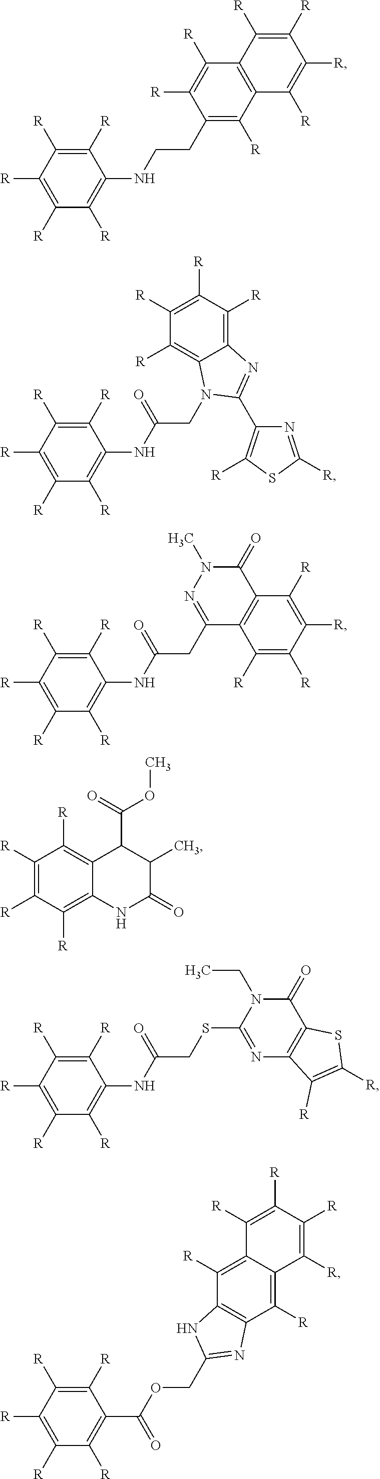

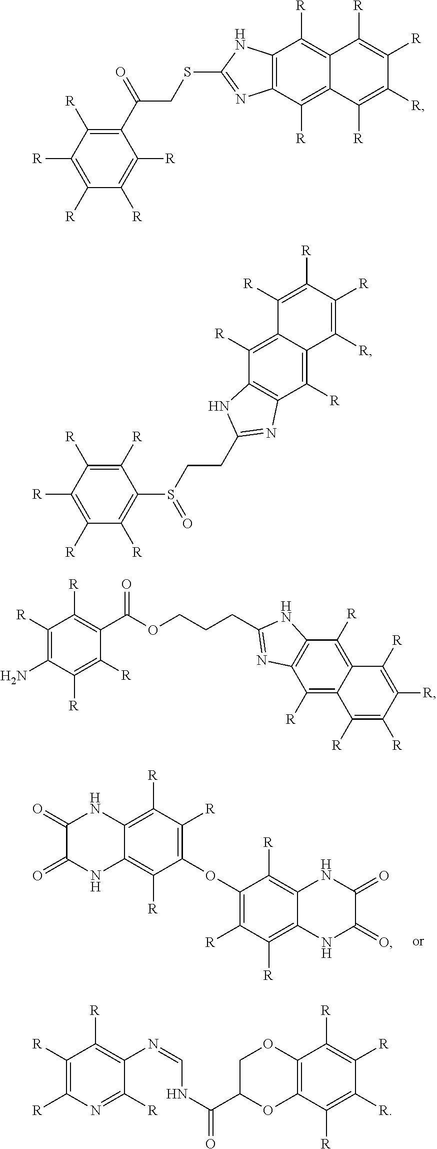

In certain embodiments, the present invention relates to the aforementioned method, wherein R is hydrogen, halogen, hydroxyl, alkoxy, nitro, amino, or amido.

In certain embodiments, the present invention relates to the aforementioned method, provided that the compound is not

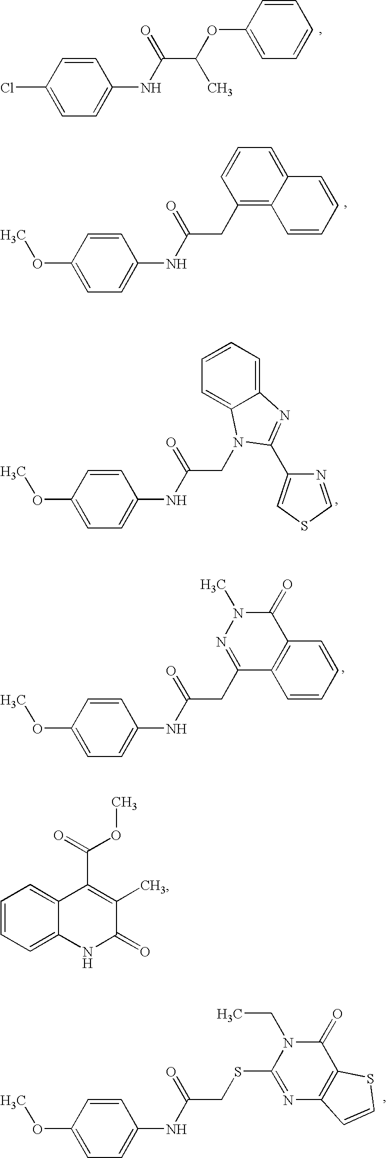

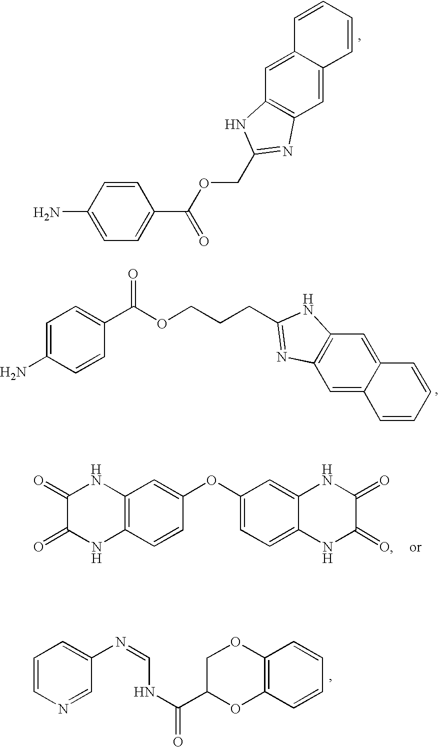

Yet another aspect of the invention relates to a method of killing or inhibiting the growth of a protozoa, comprising the step of contacting said protozoa with a compound, or a pharmaceutically acceptable salt, derivative or prodrug thereof, wherein said compound is selected from the group consisting of

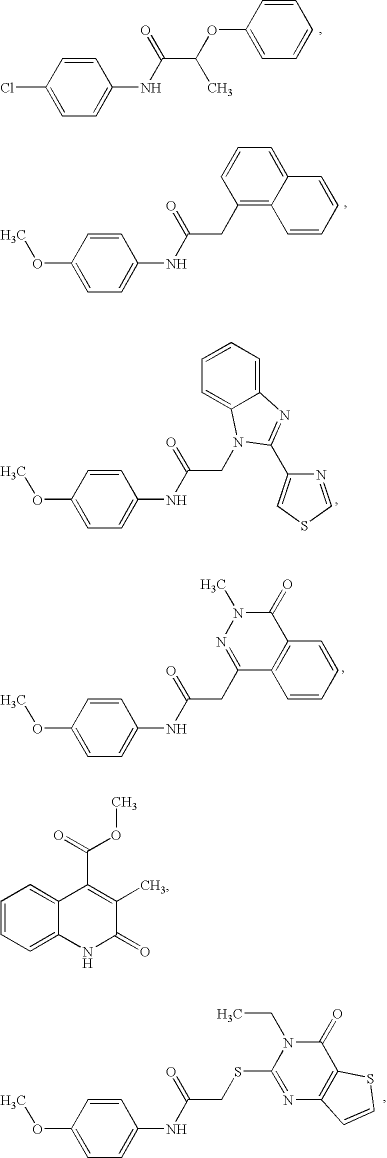

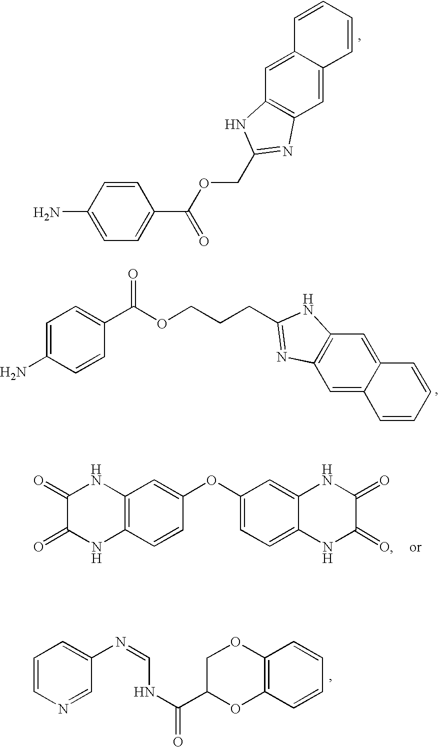

Yet another aspect of the invention relates to a method of killing or inhibiting the growth of a protozoa, comprising the step of contacting said protozoa with a compound, or a pharmaceutically acceptable salt, derivative or prodrug thereof, wherein said compound is selected from the group consisting of

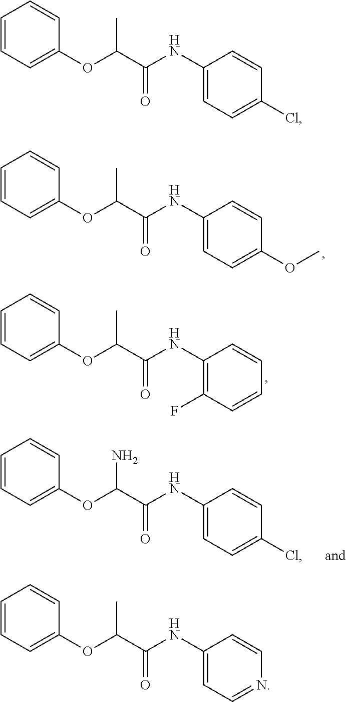

Yet another aspect of the invention relates to a method of killing or inhibiting the growth of a protozoa, comprising the step of contacting said protozoa with a compound, or a pharmaceutically acceptable salt, derivative or prodrug thereof, wherein said compound is selected from the group consisting of

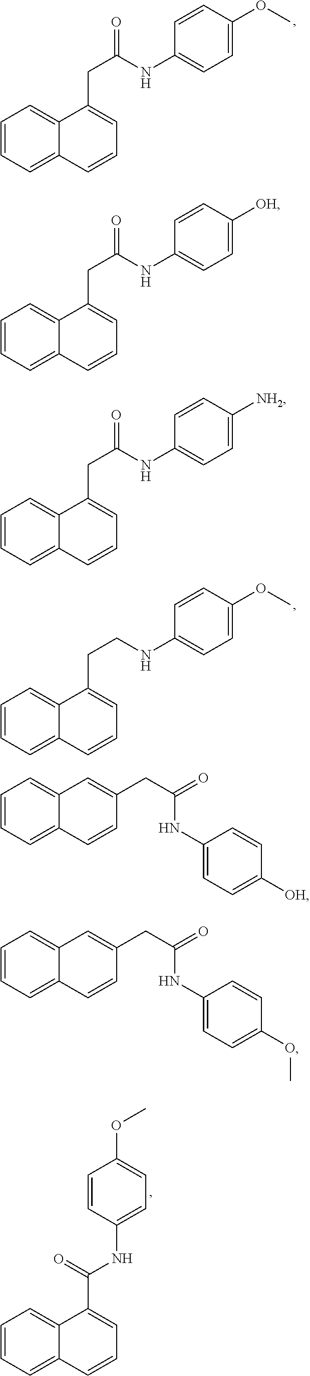

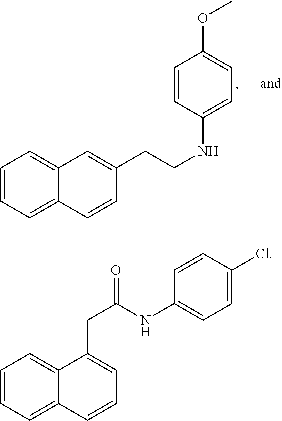

Yet another aspect of the invention relates to a method of killing or inhibiting the growth of a protozoa, comprising the step of contacting said protozoa with a compound, or a pharmaceutically acceptable salt, derivative or prodrug thereof, wherein said compound is selected from the group consisting of

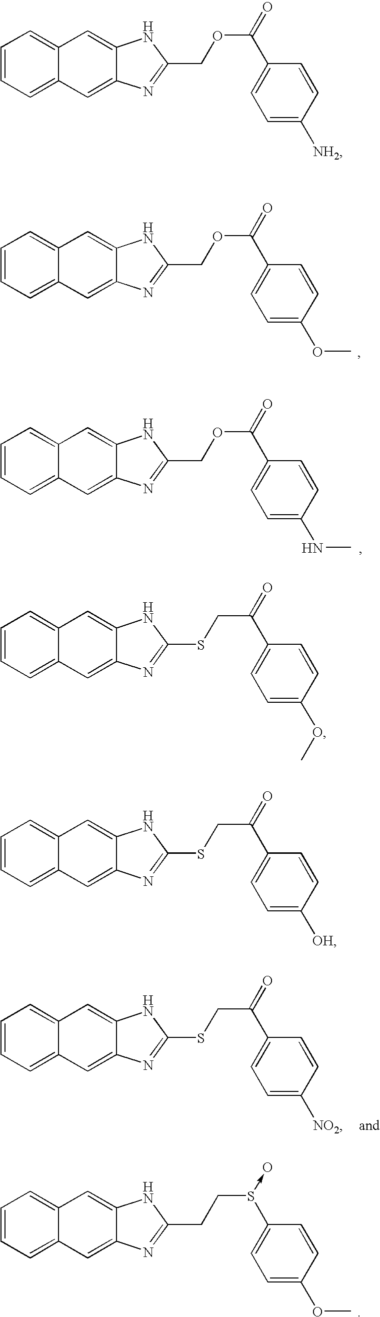

In certain embodiments, the present invention relates to the aforementioned method, wherein said protozoa is selected from the group consisting of the genera Toxoplasma, Eimeria, Cryptosporidium, Plasmodium, Babesia, Theileria, Neospora, Sarcocystis, Giardia, Entamoeba, Trichomonas, Leishmania and Trypanosoma.

In certain embodiments, the present invention relates to the method, wherein said protozoa is Cryptosporidium parvum.

According to one embodiment, the invention provides methods of treating or preventing a microbial infection in a mammal comprising the step of administering to said mammal any one of the compounds, pharmaceutical compositions or combinations described above.

According to one embodiment, the invention provides methods of treating or preventing a parasitic infection in a mammal comprising the step of administering to said mammal any one of the compounds, pharmaceutical compositions or combinations described above.

In certain embodiments, the present invention relates to the method, wherein said protozoan infection is caused by a protozoan selected from the group consisting of Toxoplasma, Eimeria, Cryptosporidium, Plasmodium, Babesia, Theileria, Neospora, Sarcocystis, Giardia, Entamoeba, Trichomonas, Leishmania and Trypanosoma.

In certain embodiments, the present invention relates to the aforementioned method, wherein said protozoan infection is caused by Cryptosporidium parvum.

The aforementioned methods may further comprise the step of co-administering to said mammal an antimicrobial agent.

In certain embodiments, the present invention relates to the aforementioned method, wherein said antimicrobial agent is an antibiotic. In certain embodiments, the present invention relates to the aforementioned method, wherein said antibiotic agent is selected from the group consisting of vancomycin, metronidazole, amoxicillin, ciprofloxacin, doxycycline, gentamicin and clindamycin.

In certain embodiments, the present invention relates to the aforementioned method, wherein said antimicrobial agent is an antifungal. In certain embodiments, the present invention relates to the aforementioned method, wherein said antifungal agent is selected from the group consisting of terbinafine, flucytosine, fluconazole, itraconazole, ketoconazole, voriconazole, nikkomycin Z, caspofungin, micafungin (FK463), anidulafungin (LY303366), amphotericin B (AmB), and nystatin.

In certain embodiments, the present invention relates to the aforementioned method, wherein said antimicrobial agent is an antiparasitic. In certain embodiments, the present invention relates to the aforementioned method, wherein said antiparasitic agent is selected from the group consisting of eflornithine, furazolidone, melarsoprol, metronidazole, ornidazole, paromomycin sulfate, pentamidine, pyrimethamine, and tinidazole.

In another embodiment, this invention provides methods of treating or preventing IMPDH mediated disease in a mammal comprising the step of administrating to said mammal any one of the compounds, pharmaceutical compositions or combinations described above. If the pharmaceutical composition only comprises the IMPDH inhibitor of this invention as the active component, such methods may additionally comprise the step of administering to said mammal an agent selected from an antiinflammatory agent, immunosuppressant, an anti-cancer agent, an anti-viral agent, or an anti-vascular hyperproliferation compound. Such additional agent may be administered to the mammal prior to, concurrently with, or following the administration of the IMPDH inhibitor composition.

In one embodiment, these methods are useful in suppressing an immune response in a mammal. Such methods are useful in treating or preventing diseases, including, transplant rejection (e.g., kidney, liver, heart, lung, pancreas (islet cells), bone marrow, cornea, small bowel and skin allografts and heart valve xenografts), graft versus host disease, and autoimmune diseases, such as rheumatoid arthritis, multiple sclerosis, juvenile diabetes, asthma, inflammatory bowel disease (Crohn's disease, ulcerative colitus), lupus, diabetes, mellitus myasthenia gravis, psoriasis, dermatitis, eczema, seborrhoea, pulmonary inflammation, eye uveitis, hepatitis, Grave's disease, Hashimoto's thyroiditis, Behcet's or Sjorgen's syndrome (dry eyes/mouth), pernicious or immunohaemolytic anaemia, idiopathic adrenal insufficiency, polyglandular autoimmune syndrome, glomerulonephritis, scleroderma, lichen planus, viteligo (depigmentation of the skin), autoimmune thyroiditis, and alveolitis.

These methods comprise the step of administering to the mammal a composition comprising an inventive compound and a pharmaceutically acceptable adjuvant. In a preferred embodiment, this particular method comprises the additional step of administering to said mammal a composition comprising an additional immunosuppressant and a pharmaceutically acceptable adjuvant.

Alternatively, this method comprises the step of administering to said mammal a composition comprising a compound of the invention; an additional immunosuppressive agent and a pharmaceutically acceptable adjuvant.

In certain embodiments, these methods are useful for inhibiting viral replication in a mammal. Such methods are useful in treating or preventing, DNA and RNA viral diseases caused by, for example, HTLV-1 and HTLV-2, HIV-1 and HIV-2, nasopharyngeal carcinoma virus, HBV, HCV, HGV, yellow fever virus, dengue fever virus, Japanese encephalitis virus, human papilloma virus, rhinoviruses and Herpes viruses, such as Epstein-Barr, cytomegaloviruses and Herpes Simplex, Types 1 and 2, or Type 6. See, U.S. Pat. No. 5,380,879 (incorporated by reference).

These methods comprise the step of administering to the mammal a composition comprising an inventive compound, and a pharmaceutically acceptable adjuvant. In a preferred embodiment, this particular method comprises the additional step of administering to said mammal a composition comprising an additional anti-viral agent and a pharmaceutically acceptable adjuvant.

Alternatively, this method comprises the step of administering to said mammal a composition comprising an inventive compound; an additional anti-viral agent and a pharmaceutically acceptable adjuvant.

In another embodiment, these methods are useful for inhibiting vascular cellular hyperproliferation in a mammal. Such methods are useful in treating or preventing diseases, including, restenosis, stenosis, artherosclerosis and other hyperproliferative vascular disease.

These methods comprise the step of administering to the mammal a composition comprising an inventive compound, and a pharmaceutically acceptable adjuvant. In a preferred embodiment, this particular method comprises the additional step of administering to said mammal a composition comprising an additional anti-vascular hyperproliferative agent and a pharmaceutically acceptable adjuvant.

Alternatively, this method comprises the step of administering to said mammal a composition comprising an inventive compound; an additional anti-vascular hyperproliferative agent and a pharmaceutically acceptable adjuvant.

In another embodiment, these methods are useful for inhibiting tumors and cancer in a mammal. Such methods are useful in treating or preventing diseases, including, tumors and malignancies, such as lymphoma, leukemia and other forms of cancer.

These methods comprise the step of administering to the mammal a composition comprising an inventive compound, and a pharmaceutically acceptable adjuvant. In a preferred embodiment, this particular method comprises the additional step of administering to said mammal a composition comprising an additional anti-tumor or anti-cancer agent and a pharmaceutically acceptable adjuvant.

Alternatively, this method comprises the step of administering to said mammal a composition comprising an inventive compound; an additional anti-tumor or anti-cancer agent and a pharmaceutically acceptable adjuvant.

In another embodiment, these methods are useful for inhibiting inflammation and inflammatory diseases in a mammal. Such methods are useful in treating or preventing diseases, including, osteoarthritis, acute pancreatitis, chronic pancreatitis, asthma and adult respiratory distress syndrome.

These methods comprise the step of administering to the mammal a composition comprising an inventive compound, and a pharmaceutically acceptable adjuvant. In a preferred embodiment, this particular method comprises the additional step of administering to said mammal a composition comprising an antiinflammatory agent and a pharmaceutically acceptable adjuvant.

Definitions. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 80 or fewer carbon atoms in its backbone (e.g., C₁-C₈₀ for straight chain, C₃-C₈₀ for branched chain), and alternatively, about 30 or fewer. Likewise, cycloalkyls have from about 3 to about 10 carbon atoms in their ring structure, and alternatively about 5, 6 or 7 carbons in the ring structure. As used herein, “fluoroalkyl” denotes an alkyl where one or more hydrogens have been replaced with fluorines.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, aromatic or heteroaromatic moieties, trifluoromethyl, cyano, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkylthio, sulfonyl, ketone, aldehyde, ester, a heterocyclyl, an aromatic or heteroaromatic moiety, trifluoromethyl, cyano, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkylthio, sulfonyl, ketone, aldehyde, ester, a heterocyclyl, an aromatic or heteroaromatic moiety, trifluoromethyl, cyano, or the like.

The term “carbocycle” is art-recognized and refers to an aromatic or non-aromatic ring in which each atom of the ring is carbon.

The terms “monocyclic,” “bicyclic,” or “tricyclic” ring systems refers to 5 or 6 member monocyclic rings, 8, 9 and 10 membered bicyclic ring structures, and 11, 12, 13 and 14 membered tricyclic ring structures, wherein each bond in each ring may be possess any degree of saturation that is chemically feasible. When such structures contain substituents, those substituents may be at any position of the ring system, unless otherwise specified. As specified, such ring systems may optionally comprise up to 4 heteroatoms selected from N, O or S. Those heteroatoms may replace any carbon atoms in these ring systems as long as the resulting compound is chemically stable.

The term “monocyclic” ring system, as used herein, includes saturated, partially unsaturated and fully unsaturated ring structures. The term “bicyclic” ring system, as used herein, includes systems wherein each ring is independently saturated, partially unsaturated and fully unsaturated. Examples of monocyclic and bicyclic ring systems useful in the compounds of this invention include, but are not limited to, cyclopentane, cyclopentene, indane, indene, cyclohexane, cyclohexene, cyclohexadiene, benzene, tetrahydronaphthalene, decahydronaphthalene, naphthalene, pyridine, piperidine, pyridazine, pyrimidine, pyrazine, 1,2,3-triazine, 1,2,4-triazine, 1,3,5-triazine, 1,2,3,4-tetrazine, 1,2,4,5-tetrazine, 1,2,3,4-tetrahydroquinoline, quinoline, 1,2,3,4-tetrahydroisoquinoline, isoquinoline, cinnoline, phthalazine, quinazoline, quinoxaline, 1,5-naphthyridine, 1,6-naphthyridine, 1,7-naphthyridine, 1,8-naphthyridine, 2,6-naphthyridine, 2,7-naphthyridine, pteridine, acridine, phenazine, 1,10-phenatroline, dibenzopyrans, 1-benzopyrans, phenothiazine, phenoxazine, thianthrene, dibenzo-p-dioxin, phenoxathiin, phenoxthionine, morpholine, thiomorpholine, tetrahydropyan, pyran, benzopyran, 1,4-dioxane, 1,3-dioxane, dihyropyridine, dihydropyran, 1-pyrindine, quinuclidine, triazolopyridine, β-carboline, indolizine, quinolizidine, tetrahydronaphtheridine, diazaphenanthrenes, thiopyran, tetrahydrothiopyran, benzodioxane, furan, benzofuran, tetrahydrofuran, pyrrole, indole, thiophene, benzothiopene, carbazole, pyrrolidine, pyrazole, isoxazole, isothiazole, imidazole, oxazole, thiazole, 1,2,3-triazole, 1,2,4-triazole, 1,2,3-oxadiazole, 1,2,4-oxadiazole, 1,3,4 oxadiazole, 1,2,5-oxadiazole, 1,2,3-thiadiazole, 1,2,4-thiadiazole, 1,3,4-thiadiazole, 1,2,5 thiadiazole, tetrazole, benzothiazole, benzoxazole, benzotriazole, benzimidazole, benzopyrazole, benzisothiazole, benzisoxazole and purine.

Additional monocyclic and bicyclic structures falling within the above description may be found in A. R. Katritzky, and C. W. Rees, eds. “Comprehensive Heterocyclic Chemistry: Structure, Reactions, Synthesis and Use of Heterocyclic Compounds, Vol. 1-8,” Pergamon Press, NY (1984), the disclosure of which is herein incorporated by reference.

It should be understood that heterocycles may be attached to the rest of the compound by any atom of the heterocycle which results in the creation of a stable structure.

The term “ring atom”, as used herein, refers to a backbone atom that makes up the ring. Such ring atoms are selected from C, N, O or S and are bound to 2 or 3 other such ring atoms (3 in the case of certain ring atoms in a bicyclic ring system). The term “ring atom” does not include hydrogen.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂—. “Halide” designates the corresponding anion of the halogens, and “pseudohalide” has the definition set forth on page 560 of “Advanced Inorganic Chemistry” by Cotton and Wilkinson, that is, for example, monovalent anionic groups sufficiently electronegative to exhibit a positive Hammett sigma value at least equaling that of a halide (e.g., CN, OCN, SCN, SeCN, TeCN, N₃, and C(CN)₃).

The terms “amine” and “amino” are art-recognized and refer to both unsubstituted and substituted amines, e.g., a moiety that may be represented by the general formulas:

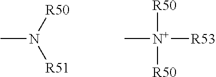

wherein R50, R51, R52 and R53 each independently represent a hydrogen, an alkyl, an alkenyl, —(CH₂)ₘ—R61, or R50 and R51 or R52, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

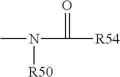

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

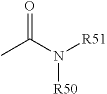

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

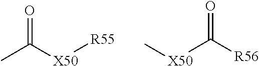

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

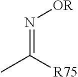

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

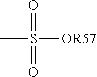

in which R57 is an electron pair, hydrogen, alkyl, cycloalkyl, or aryl.

The term “sulfate” is art recognized and includes a moiety that may be represented by the general formula:

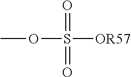

in which R57 is as defined above.

The term “sulfonamido” is art recognized and includes a moiety that may be represented by the general formula:

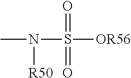

in which R50 and R56 are as defined above.

The term “sulfamoyl” is art-recognized and refers to a moiety that may be represented by the general formula:

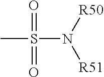

in which R50 and R51 are as defined above.

The term “sulfonyl” is art-recognized and refers to a moiety that may be represented by the general formula:

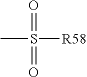

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

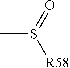

in which R58 is defined above.

The term “phosphoryl” is art-recognized and may in general be represented by the formula:

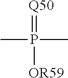

wherein Q50 represents S or O, and R59 represents hydrogen, a lower alkyl or an aryl. When used to substitute, e.g., an alkyl, the phosphoryl group of the phosphorylalkyl may be represented by the general formulas:

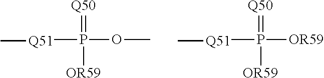

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

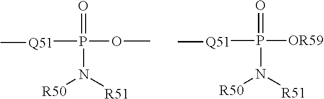

wherein Q51, R50, R51 and R59 are as defined above.

The term “phosphonamidite” is art-recognized and may be represented in the general formulas:

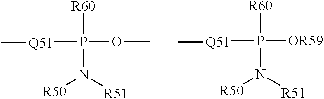

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The definition of each expression, e.g., alkyl, m, n, and the like, when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, “Handbook of Chemistry and Physics”, 67th Ed., 1986-87, inside cover.

The term “treating” as used herein refers to the alleviation of symptoms of a particular disorder in a patient or the improvement of an ascertainable measurement associated with a particular disorder. As used herein, the term “patient” refers to a mammal, including a human.

While several embodiments of the present invention are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

### EXEMPLIFICATION

The invention now being generally described, it will be more readily understood by reference to the following examples which are included merely for purposes of illustration of certain aspects and embodiments of the present invention, and are not intended to limit the invention.

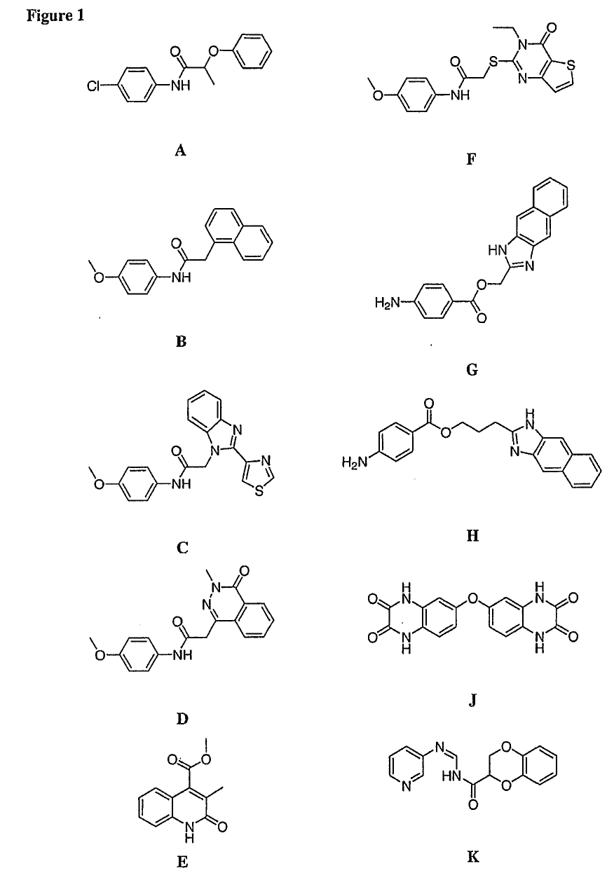

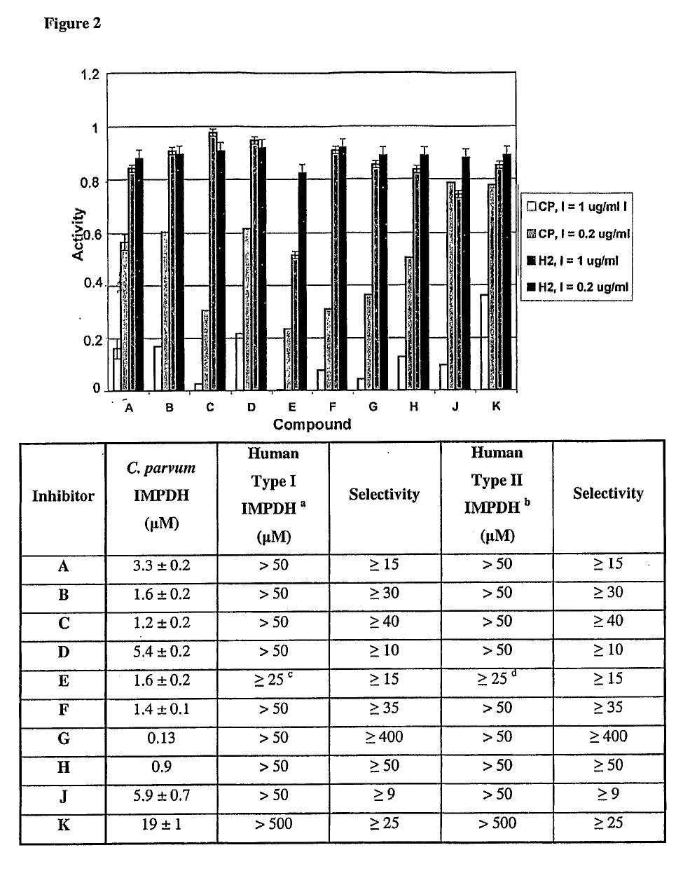

Compound libraries were screened at the National Screening Laboratory for the Regional Centers of Excellence in Biodefense and Emerging Infectious Disease (NSRB) at Harvard Medical School. The compound collection screened was provided by the National Screening Laboratory for the Regional Centers of Excellence in BioDefense and Emerging Infectious Disease (NSRB) at Harvard Medical School. The collection is from commercial sources, known bioactive collections, and natural product extracts. The initial screen consisted of testing 44,000 compounds from which cherry picks were selected for further in-house testing. Ten compounds were identified that specifically inhibit C. parvum IMPDH but not human IMPDH type I and type II (FIGS. 1 and 2).

Recombinant C. parvum IMPDH was expressed in bacteria and purified as described previously [N. N. Umejiego et al, J Biol Chem 279, 40320-40327 (2004)].

Initial Screen. A detailed protocol is provided below, we first add assay buffer, enzyme, screening compounds, and control compounds to the assay plate wells. Then, a background absorbance reading of each assay plate is taken at 340 nm. Substrate (IMP and NAD) is added to start the enzyme reaction, which is allowed to proceed for 3 hours. At this point, GMP—an inhibitor of the IMPDH enzyme—is added to quench the reaction. The assay plates are then immediately read at 340 nM to get the final reaction endpoint.

Protocol for Screen 449. Inhibition of Cryptosporidium parvum Inosine-5′-monophosphate dehydrogenase.
1. Add 30 μl of Enzyme assay mix to plate* wells (Approx. 1 hr 15 mins for 70 plates) *Corning 3640: 384 well, Clear Flat Bottom Polystyrene Non-binding Surface Microplate Final assay condition is 50 mM Tris, pH 8, 100 mM KCl, 3 mM EDTA, 1 mM DTT, 250 μM IMP, and 500 μM NAD.
2. Pin transfer 100 nl of potential inhibitor compounds to experiment wells in assay plate (Approx. 1 hr. 30 mins for 70 plates)
3. Add positive and negative controls to control wells in assay plates using multi-pipetter (Approx. 1 hr for 70 plates)
4. Read absorbance at 340 nm of all wells per plate using the Envision plate reader (Approx. 1 hr. 30 mins for 70 plates)
5. Add 40 ul of substrate IMP/NAD mix to start reaction (Approx. 1 hr for 70 plates)
6. After 3 hrs, add 10 μl of >200 mM GMP to each well to quench reaction (Approx. 1 hr for 70 plates)
7. Read absorbance at 340 nm of all wells per plate using the Envision plate reader (Approx. 1 hr. 30 mins for 70 plates)

Analysis entails first subtracting the initial background absorbance readout from the final absorbance readout. The values for the negative control wells are computed to determine the average change in absorbance for the 3 hour reaction; this serves as a reference point for the experimental assay well analysis. For a negative control well, no screening compound is added to the plate well. For a positive control well, 5 ul of either 100 mM GMP or >200 mM GMP is added to the plate well.

Analysis of experimental wells further entails comparing the change in absorbance for the experimental wells to the average change in absorbance for the negative control wells, and determining a z-score and a percent inhibition. The z-score was not factored into the criteria for determining screening hits because the values tended to fluctuate for any given experimental plate. The screening hit limit was set at 45% inhibition.

The strength of screening positives were not defined by any strict cut-off ranges, however strong hits do generally correlate with greater than 70% inhibition, medium hits with between 70% and 60% inhibition, and weak hits with between 60% and 45% inhibition. The strength of screening positives does in fact reflect our degree of confidence in the positive and not just its potency.

Negative controls consistently showed no inhibition in comparison to the experimental wells. Positive controls on the other had showed inhibition consistent with how much inhibitor was added to the wells.

The number of cherry picks (134) was limited by the 0.3% cherry pick limit, which also factored into setting the screen hit limit. Some compounds hit in other researcher's screens but were cherry picked anyway due to the degree of potency. The NSRB chemistry group was not consulted for the preliminary analysis of screening positives. The secondary screen involves re-screening the cherry-picks against C. parvum IMPDH and the human IMPDH.

Rescreening Hit Compounds. Two sets of master plates were prepared for both the C. parvum and human type II IMPDH enzymes consisting only of the compounds identified in the initial screen as potential inhibitors. The inhibitors were tested at concentrations of 1 μg/mL and 0.2 μg/mL. Assays were performed in 100 mM KCl, 3 mM EDTA, 1 mM dithiothreitol, and 50 mM Tris, pH 8 (assay buffer) at 25° C. using a CytoFlour Multi-Well Plate Reader Series 4000. The C. parvum plates were assayed in the presence of 70 nM C. parvum IMPDH, 500 μM NAD⁺ and 250 μM IMP. The human type II plates were assayed in the presence of 70 nM human type II IMPDH, 100 μM NAD⁺ and 125 μM IMP. The production of NADH was monitored at 340 nm (ε=6.22 mM⁻¹ cm⁻¹) at 25° C. using a CytoFlour® Multi-Well Plate Reader Series 4000. The inhibition of the parasite enzyme was confirmed, and the selective nature of the compounds was demonstrated.

Determining the IC₅₀ values. Inhibitors at varying concentrations (0.005 μM-100 μM) were incubated with 52 nM C. parvum in assay buffer for 10 min at room temperature. The reaction was initiated by the addition of NAD and IMP for final concentrations of 300 μM and 150 μM, respectively. Selectivity was measured against human type II and T. foetus IMPDH at 25° C. in assay buffer. The former was assayed in the presence of 300 μM NAD⁺, 40 μM IMP and 160 nM human type II IMPDH, and the latter in the presence of 300 μM NAD⁺, 20 μM IMP and 28 nM T. foetus IMPDH. The production of NADH was monitored spectrophotometrically at 340 nm (=6.22 mM⁻¹ cm⁻¹) using a Hitachi U-2000 spectrophotometer.

IC₅₀ values were calculated for each inhibitor according to the following equation: νᵢ=νₒ/(1+[I]/IC₅₀), using the SigmaPlot program (SPSS, Inc.).

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-61. (canceled)

62. A method of killing or inhibiting the growth of a protozoa comprising the step of contacting said protozoa with a compound, or a pharmaceutically acceptable salt, derivative or prodrug thereof, wherein said compound is selected from the group consisting of 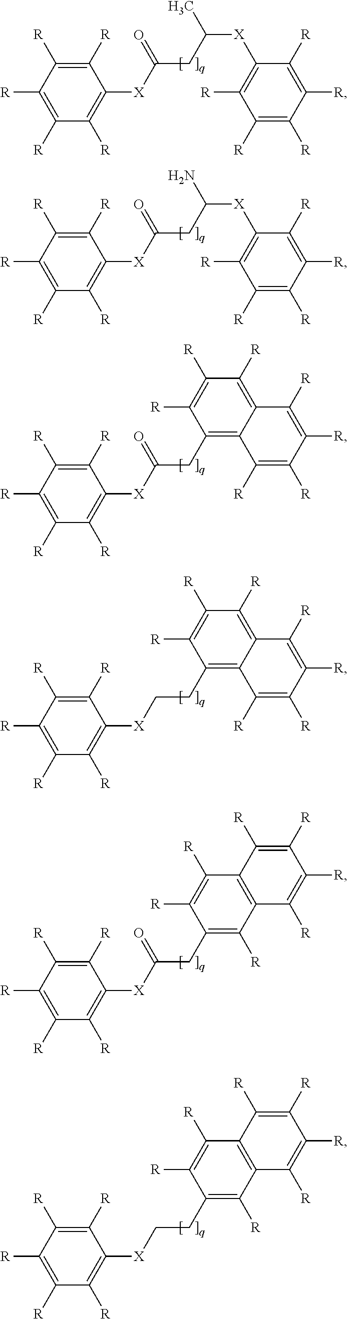 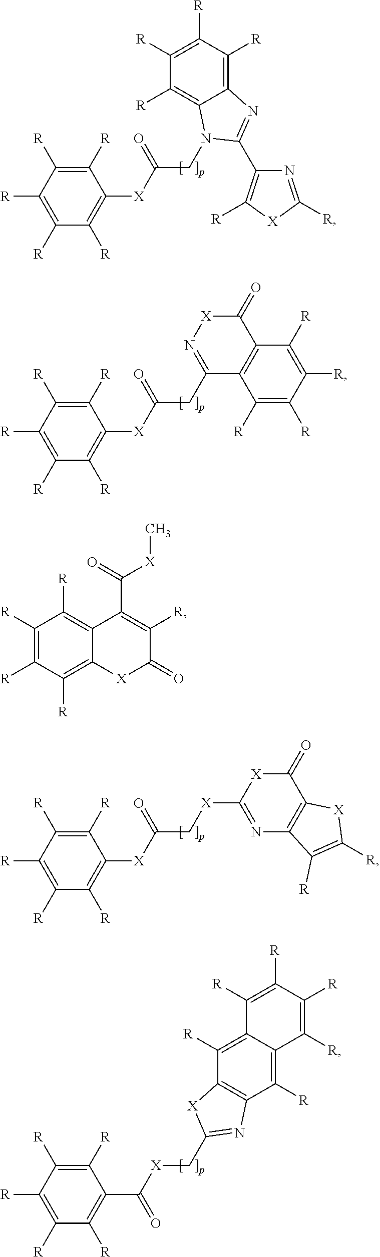 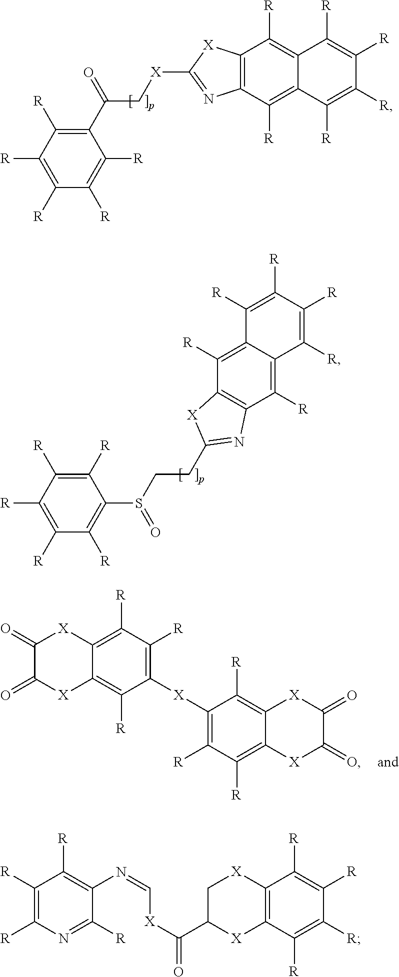
wherein, independently for each occurrence,
X is —CH₂—, —N(R^{N})—, —O—, or —S—;
p is 1-4 inclusive;
q is 0-3 inclusive;
R^{N} is hydrogen, alkyl, aralkyl, or carbonyl;
R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and
R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

63. The method of claim 62, wherein X is —N(H)—.

64. The method of claim 62, wherein X is —O—

65. The method of claim 62, wherein X is —CH₂—.

66. The method of claim 62, wherein p is 1.

67. The method of claim 62, wherein p is 2.

68. The method of claim 62, wherein q is 0.

69. The method of claim 62, wherein q is 1.

70. The method of claim 62, wherein R is hydrogen, halogen, hydroxyl, alkoxy, amino, or amido.

71. The method of claim 62, wherein said compound is selected from the group consisting of

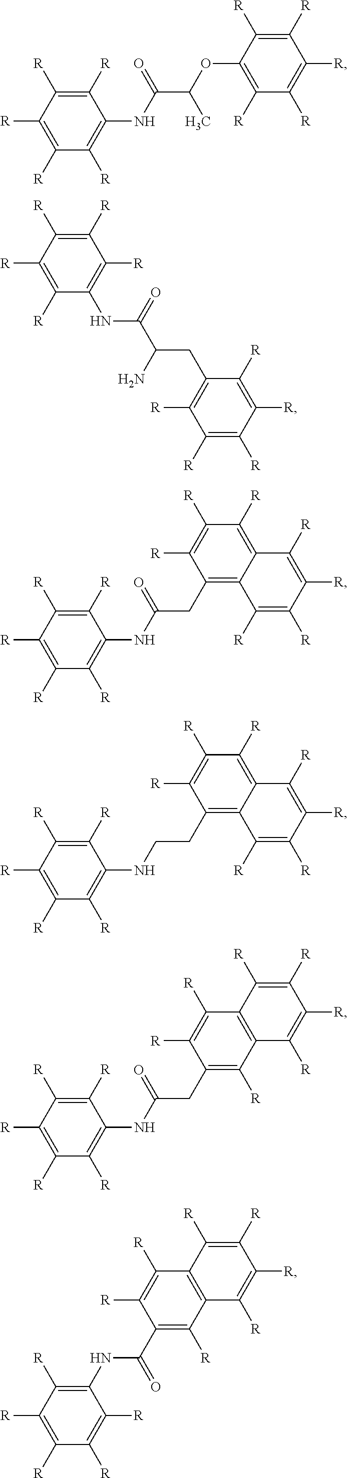

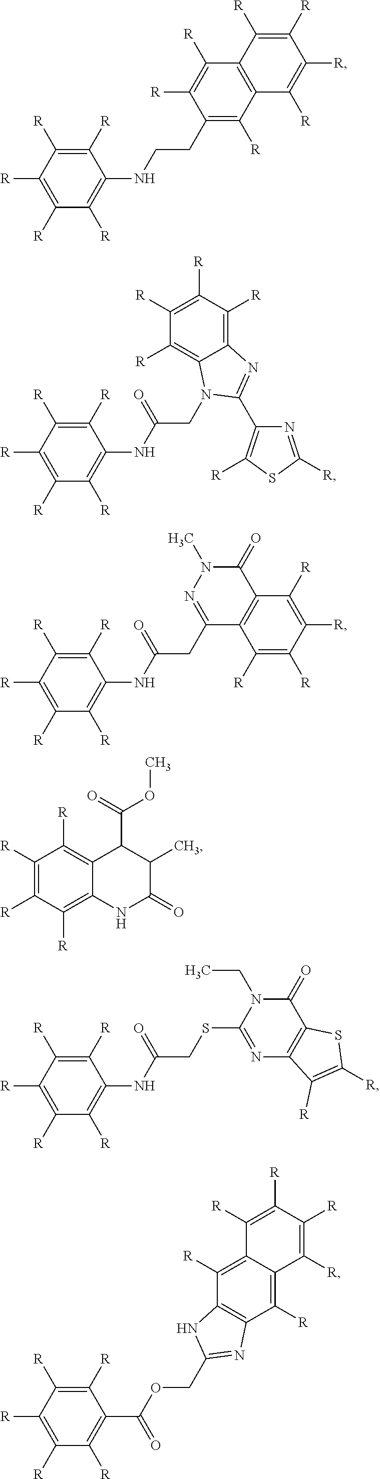

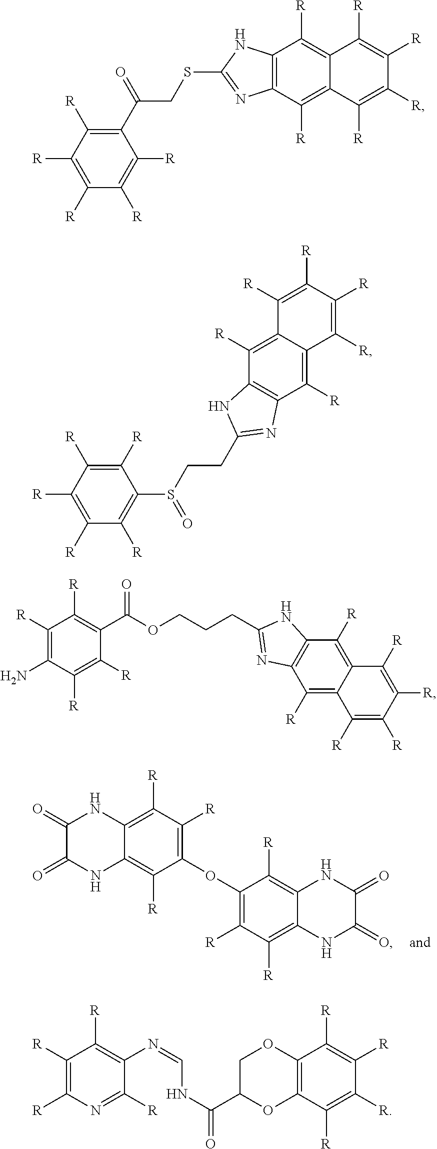

72. The method of claim 71, wherein R is hydrogen, halogen, hydroxyl, alkoxy, nitro, amino, or amido.

73. The method of claim 71, wherein said compound is selected from the group consisting of

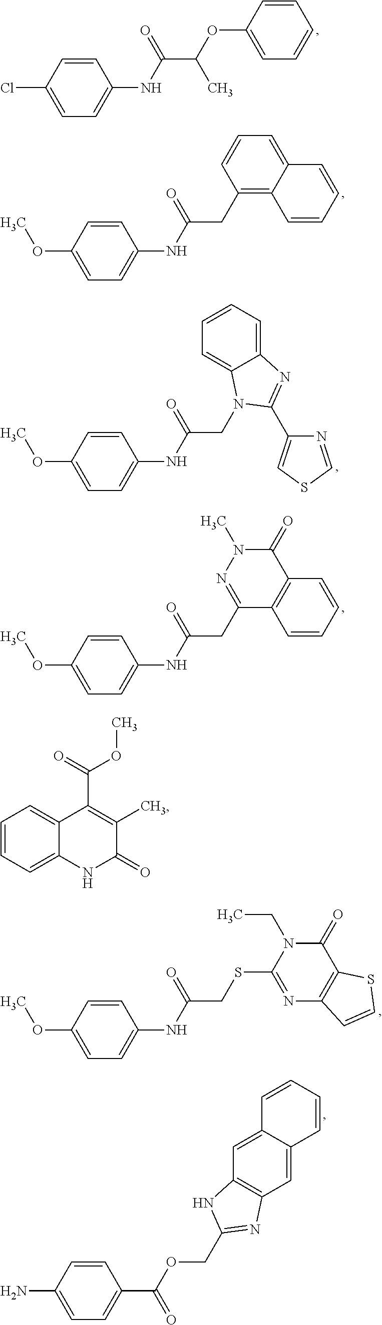

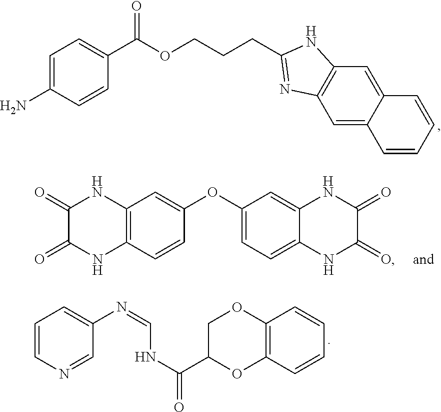

74. The method of claim 71, wherein said compound is selected from the group consisting of

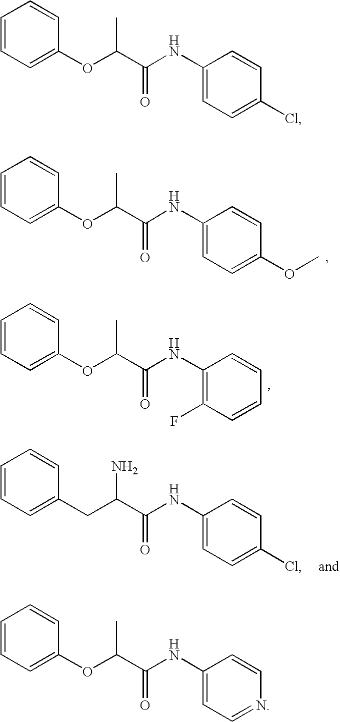

75. The method of claim 71, wherein said compound is selected from the group consisting of

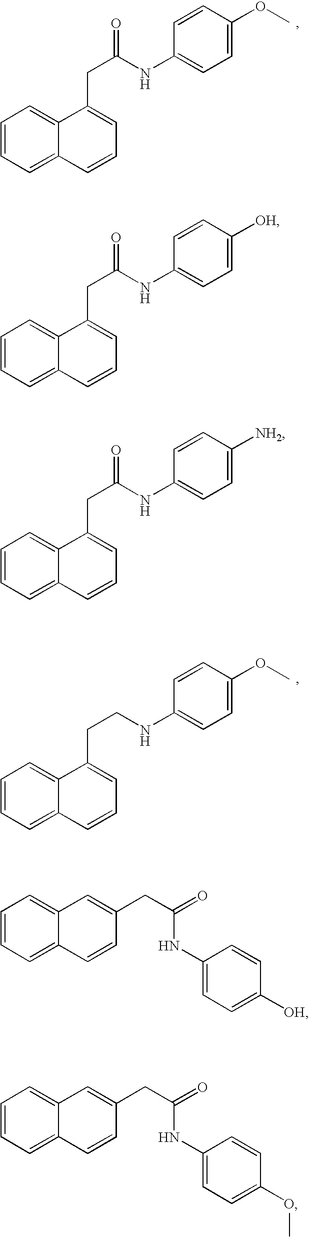

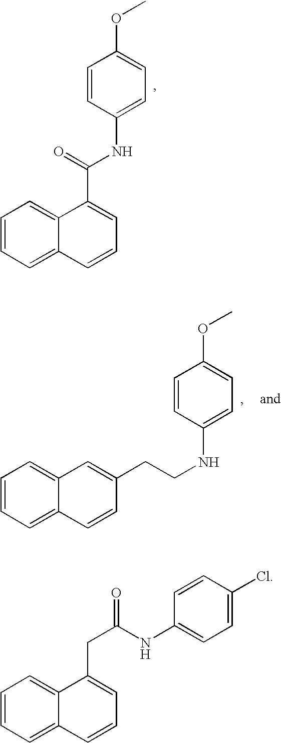

76. The method of claim 71, wherein said compound is selected from the group consisting of

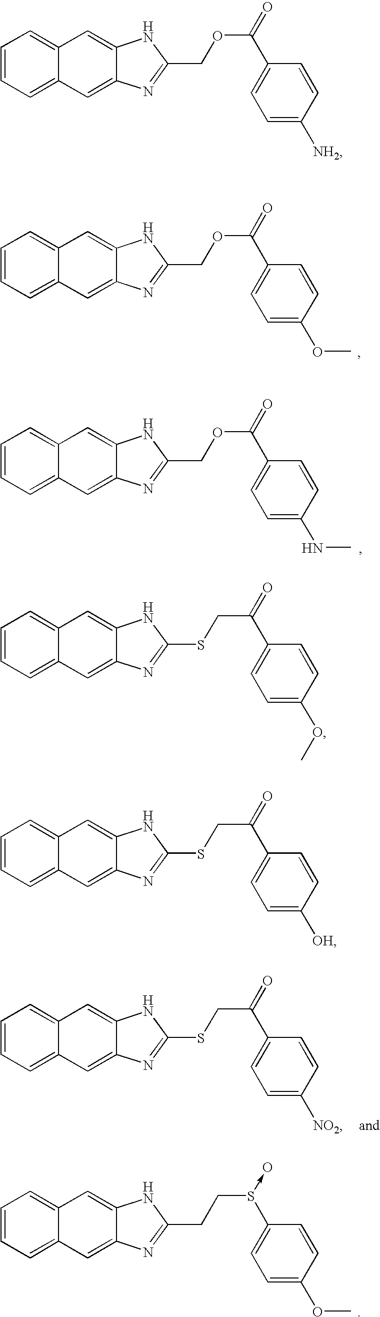

77. The method of claim 62, wherein said protozoa is selected from the group consisting of the genera Toxoplasma, Eimeria, Cryptosporidium, Plasmodium, Babesia, Theileria, Neospora, Sarcocystis, Giardia, Entamoeba, Trichomonas, Leishmania and Trypanosoma.

78. The method of claim 62, wherein said protozoa is Cryptosporidium parvum.

79-139. (canceled)

140. A pharmaceutical composition, comprising a pharmaceutically acceptable carrier, adjuvant or vehicle and at least one compound, or a pharmaceutically acceptable salt, derivative or prodrug thereof, selected from the group consisting of 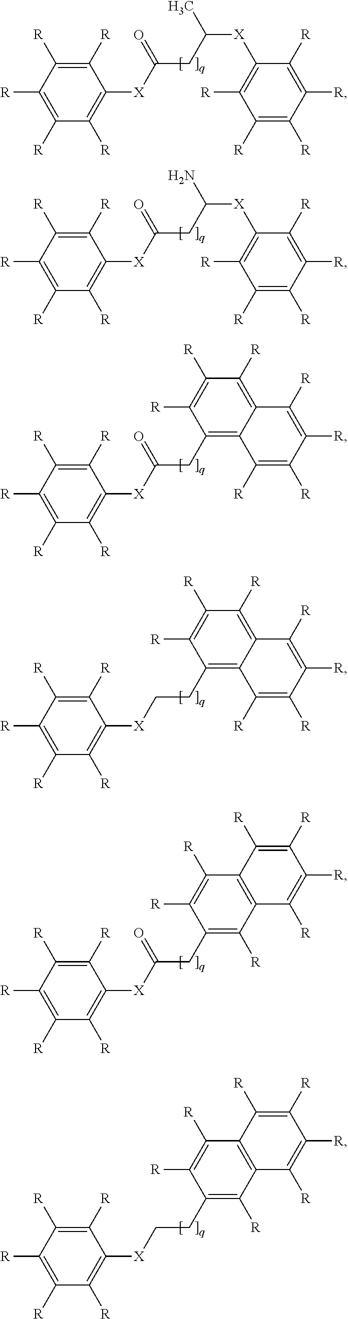 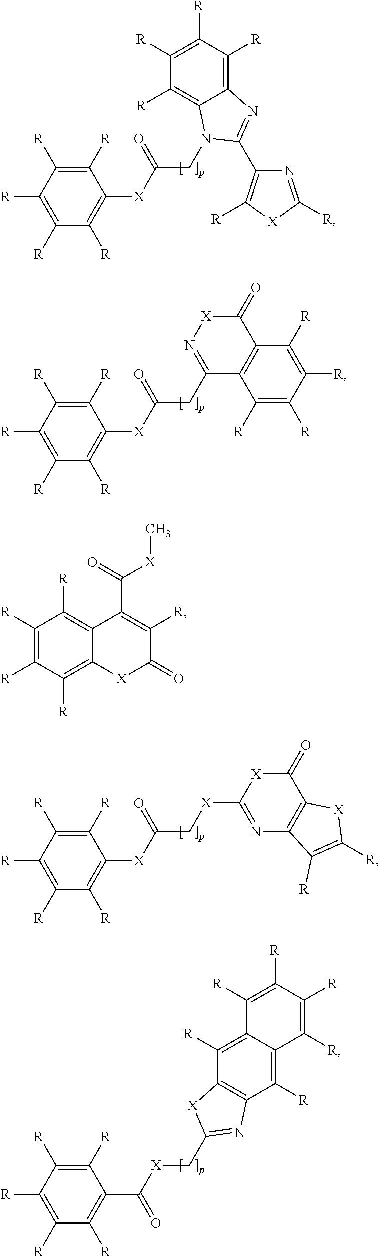 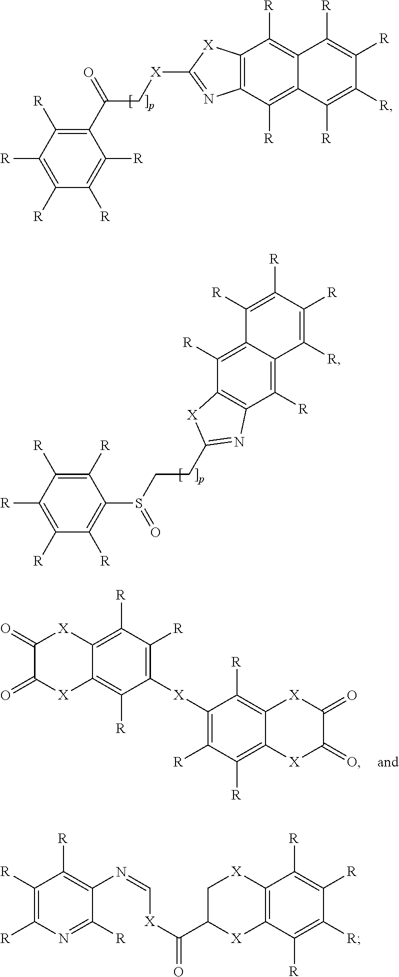
wherein, independently for each occurrence,
X is —CH₂—, —N(R^{N})—, —O—, or —S—;
p is 1-4 inclusive;
q is 0-3 inclusive;
R^{N} is hydrogen, alkyl, aralkyl, or carbonyl;
R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and
R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

141-231. (canceled)

232. A compound, or a pharmaceutically acceptable salt, derivative or prodrug thereof, selected from the group consisting of 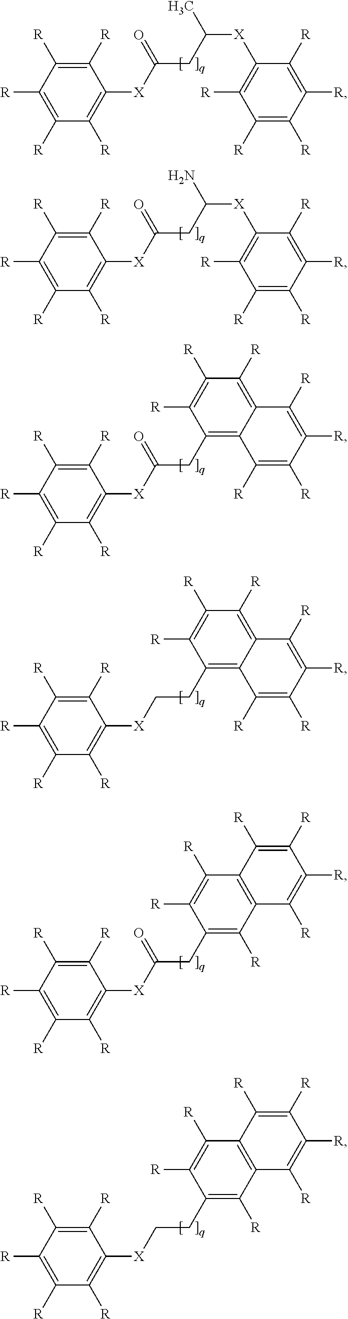 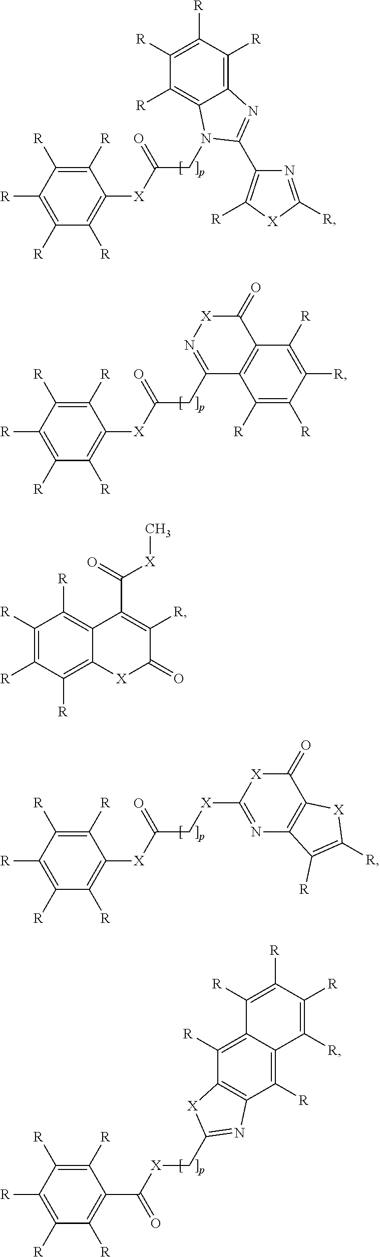 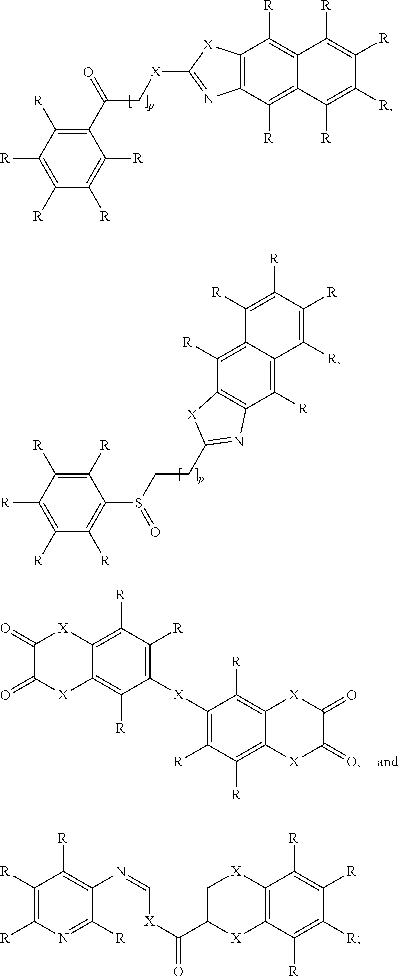
wherein, independently for each occurrence,
X is —CH₂—, —N(R^{N})—, —O—, or —S—;
p is 1-4 inclusive;
q is 0-3 inclusive;
R^{N} is hydrogen, alkyl, aralkyl, or carbonyl;
R is hydrogen, halogen, azide, alkyl, aralkyl, alkenyl, alkynyl, cycloalkyl, hydroxyl, alkoxy, aryloxy, heteroaryloxy, amino, alkylamino, arylamino, heteroarylamino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, cyano, or —(CH₂)ₙR^{C}; and
R^{C} is hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, trifluoromethyl, or cyano.

233-247. (canceled)
